SEQUENCE LISTING (14 sequences)

SEQ ID NO: 1 - protein, 9 aa, Homo sapiens
DAHKSEVAH

SEQ ID NO: 2 - protein, 609 aa, Homo sapiens [sequence omitted]

SEQ ID NO: 3 - protein, 476 aa, Homo sapiens
MAKAAAIGIDLGTTYSCVGVFQHGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 4 - protein, 642 aa, Drosophila melanogaster
MPAIGIDLGTTYSCVGVYQHGKVEIIANDQGNRTTPSYVAFTDSERLIGDPAKNQVAMNPRNTVFDAKRLIGRKYDDPKIAEDMKHWPFKVVSDGGKPKIGVEYKGESKRFAPEEISSMVLTKMKETAEAYLGESITDAVITVPAYFNDSQRQATKDAGHIAGLNVLRIINEPTAAALAYGLDKNLKGERNVLIFDLGGGTFDVSILTIDEGSLFEVRSTAGDTHLGGEDFDNRLVTHLADEFKRKYKKDLRSNPRALRRLRTAAERAKRTLSSSTEATIEIDALFEGQDFYTKVSRARFEELCADLFRNTLQPVEKALNDAKMDKGQIHDIVLVGGSTRIPKVQSLLQDFFHGKNLNLSINPDEAVAYGAAVQAAILSGDQSGKIQDVLLVDVAPLSLGIETAGGVMTKLIERNCRIPCKQTKTFSTYADNQPGVSIQVYEGERAMTKDNNALGTFDLSGIPPAPRGVPQIEVTFDLDANGILNVSAKEMSTGKAKNITIKNDKGRLSQAEIDRMVNEAEKYADEDEKHRQRITSRNALESYVFNVKQAVEQAPAGKLDEADKNSVLDKCNDTIRWLDSNTTAEKEEFDHKLEELTRHCSPIMTKMHQQGAGAGAGGPGANCGQQAGGFGGYSGPTVEEVD

SEQ ID NO: 5 - protein, 649 aa, Saccharomyces cerevisiae
MSRAVGIDLGTTYSCVAHFSNDRVEIIANDQGNRTTPSYVAFTDTERLIGDAAKNQAAINPHNTVFDAKRLIGRKFDDPEVTTDAKHFPFKVISRDGKPVVQVEYKGETKTFTPEEISSMVLSKMKETAENYLGTTVNDAVVTVPAYFNDSQRQATKDAGTIAGMNVLRIINEPTAAAIAYGLDKKGRAEHNVLIFDLGGGTFDVSLLSIDEGVFEVKATAGDTHLGGEDFDNRLVNHLATEFKRKTKKDISNNQRSLRRLRTAAERAKRALSSSSQTSIEIDSLFEGMDFYTSLTRARFEELCADLFRSTLEPVEKVLKDSKLDKSQIDEIVLVGGSTRIPKIQKLVSDFFNGKEPNRSINPDEAVAYGAAVQAAILTGDQSTKTQDLLLLDVAPLSLGIETAGGIMTKLIPRNSTIPTKKSETFSTYADNQPGVLIQVFEGERTRTKDNNLLGKFELSGIPPAPRGVPQIDVTFDIDANGILNVSALEKGTGKSNKITITNDKGRLSKDDIDRMVSEAEKYRADDEREAERVQAKNQLESYAFTLKNTINEASFKEKVGEDDAKRLETASQETIDWLDASQAASTDEYKDRQKELEGIANPIMTKFYGAGAGAGPGAGESGGFPGSMPNSGATGGGEDTGPTVEEVD

SEQ ID NO: 6 - protein, 649 aa, Oryza sativa
MAGKGEGPAIGIDLGTTYSCVGVWQHDRVEIIANDQGNRTTPSYVGFTDSERLIGDAAKNQVAMNPINTVFDAKRLIGRRFSDASVQSDIKLWPFKVIAGPGDKPMIVVQYKGEEKQFAAEEISSMVLIKMREIAEAYLGTTIKNAVVTVPAYFNDSQRQATKDAGVIAGLNVMRIINEPTAAAIAYGLDKKATSVGEKNVLIFDLGGGTFDVSLLTIEEGIFEVKATAGDTHLGGEDFDNRMVNHFVQEFKRKNKKDITGNPRALRRLRTACERAKRTLSSTAQTTIEIDSLYEGIDFYSTITRARFEELNMDLFRKCMEPVEKCLRDAKMDKSSVHDVVLVGGSTRIPRVQQLLQDFFNGKELCKNINPDEAVAYGAAVQAAILSGEGNEKVQDLLLLDVTPLSLGLETAGGVMTVLIPRNTTIPTKKEQVFSTYSDNQPGVLIQVYEGERTRTRDNNLLGKFELSGIPPAPRGVPQITVCFDIDANGILNVSAEDKTTGQKNKITITNDKGRLSKEEIEKMVQEAEKYKSEDEEHKKKVESKNALENYAYNMRNTIKDEKIASKLPAADKKKIEDAIDQAIQWLDGNQLAEADEFDDKMKELEGICNPIIAKMYQGAGADMAGGMDEDDAPPAGGSGAGPKIEEVD

SEQ ID NO: 7 - protein, 653 aa, Oryza sativa
MAGNKGEGPAIGIDLGTTYSCVGVWQHDRVEIIANDQGNRTTPSYVAFTDTERLIGDAAKNQVAMNPTNTVFDAKRLIGRRFSDPSVQADMKMWPFKVVPGPADKPMIVVTYKGEEKKFSAEEISSMVLTKMKEIAEAFLSTTIKNAVITVPAYFNDSQRQATKDAGVISGLNVMRIINEPTAAAIAYGLDKKAASTGEKNVLIFDLGGGTFDVSILTIEEGIFEVKATAGDTHLGGEDFDNRMVNHFVQEFKRKHKKDITGNPRALRRLRTACERAKRTLSSTAQTTIEIDSLYEGIDFYATITRARFEELNMDLFRRCMEPVEKCLRDAKMDKAQIHDVVLVGGSTRIPKVQQLLQDFFNGKELCKSINPDEAVAYGAAVQAAILSGEGNQRVQDLLLLDVTPLSLGLETAGGVMTVLIPRNTTIPTKKEQVFSTYSDNQPGVLIQVYEGERTRTKDNNLLGKFELTGIPPAPRGVPQINVTFDIDANGILNVSAEDKTTGKKNKITITNDKGRLSKEEIERMVQEAEKYKAEDEQVRHKVEARNALENYAYNMRNTVRDEKIASKLPADDKKKIEDAIEDAIKWLDGNQLAEADEFEDKMKELESLCNPIISKMYQGGAGGPAGMDEDAPNGSAGTGGGSGAGPKIEEVD

SEQ ID NO: 8 - protein, 648 aa, Nicotiana tabacum
MAGKGEGPAIGIDLGTTYSCVGVWQHDRVEIIANDQGNRTTPSYVGFTDSERLIGDAAKNQVAMNPINTVFDAKRLIGRRFSDASVQSDIKLWPFKVISGPGDKPMIVVNYKGEEKQFAAEEISSMVLIKMKEIAEAFLGSTVKNAVVTVPAYFNDSQRQATKDAGVISGLNVMRIINEPTAAAIAYGLDKKATSVGEKNVLIFDLGGGTFDVSLLTIEEGIFEVKATAGDTHLGGEDFDNRMVNHFVQEFKRKHKKDITGNPRALRRLRTACERAKRTLSSTAQTTIEIDSLYEGVDFYSTITRARFEELNMDLFRKCMEPVEKCLRDAKMDKSTVHDVVLVGGSTRIPKVQQLLQDFFNGKELCKSINPDEAVAYGAAVQAAILSGEGNEKVQDLLLLDVTPLSLGLETAGGVMTVLIPRNTTIPTKKEQVFSTYSDNQPGVLIQVYEGERARTRDNNLLGKFELSGIPPAPRGVPQITVCFDIDANGILNVSAEDKTTGQKNKITITNDKGRLSKEEIEKMVQEAEKYKAEDEEHKKKVEAKNALENYAYNMRNTIKDEKIGSKLSSDDKKKIEDAIDQAISWLDSNQLAEADEFEDKMKELESICNPIIAKMYQGAGGEAGAPMDDDAPPAGGSSAGPKIEEVD

SEQ ID NO: 9 - protein, 69 aa, Oryza sativa
ATAGDTHLGGEDFDNRVVPGPADKSPMIVVTYKGEEKNAVITVPAYFNDSQRIINEPTAAAIAYGLDKK

SEQ ID NO: 10 - protein, 93 aa, Oryza sativa
NQAAVNPERNGHVEIIANDQGNRIVNKDGKPYIQVKIINEPTAAAIAYGLDKKKLGTVIGIDLGTTYSCVGVYKVEIESLFDGTDSFSEPLTR

SEQ ID NO: 11 - protein, 56 aa, Oryza sativa
NQADSVVYQTEKKQDITITGASTLPKDEVERDVVLLDVTPLSLSLGLETLGGVMTK

SEQ ID NO: 12 - protein, 653 aa, Oryza sativa
MAGNKGEGPAIGIDLGTTYSCVGVWQHDRVEIIANDQGNRTTPSYVAFTDTERLIGDAAKNQVAMNPTNTVFDAKRLIGRRFSDPSVQADMKMWPFKVVPGPADKPMIVVTYKGEEKKFSAEEISSMVLTKMKEIAEAFLSTTIKNAVITVPAYFNDSQRQATKDAGVISGLNVMRIINEPTAAAIAYGLDKKAASTGEKNVLIFDLGGGTFDVSILTIEEGIFEVKATAGDTHLGGEDFDNRMVNHFVQEFKRKHKKDITGNPRALRRLRTACERAKRTLSSTAQTTIEIESLYEGIDFYATITRARFEELNMDLFRRCMEPVEKCLRDAKMDKAQIHDVVLVGGSTRIPKVQQLLQDFFNGKELCKSINPDEAVAYGAAVQAAILSGEGNQRVQDLLLLDVTPLSLGLETAGGVMTVLIPRNTTIPTKKEQVFSTYSDNQPGVLIQVYEGERTRTKDNNLLGKFELTGIPPAPRGVPQINVTFDIDANGILNVSAEDKTTGKKNKITITNDKGRLSKEEIERMVQEAEKYKAEDEQVRHKVEARNALENYAYNMRNTVRDEKIASKLPADDKKKIEDAIEDAIKWLDGNQLAEADEFEDKMKELESLCNPIISKMYQGGAGGPAGMDEDAPNGSAGTGGGSGAGPKIEEVD

SEQ ID NO: 13 - protein, 663 aa, Oryza sativa
MDRVRGSAFLLGVLLAGSLFAFSVAKEETKKLGTVIGIDLGTTYSCVGVYKNGHVEIIANDQGNRITPSWVAFTDSERLIGEAAKNQAAVNPERTIFDVKRDIGRKFEEKEVQRDMKLVPYKIVNKIGKPYIQVKIKDGENKVFSPEEVSAMILGKMKETAEAYLGKKINDAVVTVPAYFNDAQRQATKDAGVIAGLNVARIINEPTAAAIAYGLDKKGGEKNILVFDLGGGTFDVSILTIDNGVFEVLATNGDTHLGGEDFDQRIMEYFIKLIKKKYSKDISKDNRALGKLRREAERAKRALSNQHQVRVEIESLFDGTDFSEPLTRARFEELNNDLFRKTMGPVKKAMDDAGLEKSQIHEIVLVGGSTRIPKVQQLLRDYFEGKEPNKGVNPDEAVAYGAAVQGSILSGEGGDETKDILLLDVAPLTLGIETVGGVMTKLIPRNTVIPTKKSQVFTTYQDQQTTVSIQVFEGERSMTKDCRLLGKFDLSGIPAAPRGTPQIEVTFEVDANGILNVKAEDKGTGKSEKITITNEKGRLSQEEIDRMVREAEEFAEEDKKVKERIDARNQLETYVYNMKNTVGDKDKLADKLESEEKEKVEEALKEALEWLDENQTAEKEEYEEKLKEVEAVCNPIISAVYQRTGGAPGGRRRGRLDDEHDEL

SEQ ID NO: 14 - protein, 715 aa, Oryza sativa
MASFTSQLGAMACGAAPSTSPLAARRSGQLFVGRKPAAASVQMRVPRAGRARGVAMRVACEKVVGIDLGTTNSAVAAMEGGKPTVITNAEGQRTTPSVVAYTKGGERLVGQIAKRQAVVNPENTFFSVKRFIGRKMAEVDDEAKQVSYHVVRDDNGNVKLDCPAIGKQFAAEEISAQVLRKLVDDASKFLNDKITKAVVTVPAYFNDSQRTATKDAGRIAGLEVLRIINEPTAASLAYGFEKKNNETILVFDLGGGTFDVSVLEVGDGVFEVLSTSGDTHLGGDDFDKFYFCWVFYFGAMTHETPKVVDWLASNFKKDEGIDLLKDKQALQRLTEAAEKAKMELSTLSQTNISLPFITATADGPKHIETTLSRAKFEELCSDLIDRLKTPVTNALRDAKLSVDNLDEVILVGGSTRIPSVQELVKKITGKDPNVTVNPDEVVSLGAAVQGGVLAGDVKDVVLLDVTPLSLGLETLGGVMTKIIPRNTTLPTSKSEVFSTAADGQTSVEINVLQGEREFVRDNKSLGSFRLDGIPPAPRGVPQIEVKFDIDANGILSVAAIDKGTGKKQDITITGASTLPKDEVERMVEEADKFAQEDKEKRDAIDTKNQADSVVYQTEKQLKELGDKVPAPVKEKVDAKLNELKEAIAGGSTQSMKDAMAALNEEVMQIGQAMYNQQPNAGAAGPTPGADAGPTSSGGKGPNDGDVIDADFTDSN

(19) United States
(12) Patent Application Publication
(10) Pub. No.: US 2013/0157356 A1
(43) Pub. Date: Jun. 20, 2013

(54) METHODS & COMPOSITIONS FOR IMPROVING PROTEIN PRODUCTION

(71) Applicants: Michael E. Barnett, Manhattan, KS (US); Matthew S. Croughan, Claremont, CA (US)

(72) Inventors: Michael E. Barnett, Manhattan, KS (US); Matthew S. Croughan, Claremont, CA (US)

(73) Assignee: VENTRIA BIOSCIENCE, Sacramento, CA (US)

(21) Appl. No.: 13/575,131

(22) PCT Filed: Jan. 24, 2011
(86) PCT No.: PCT/US11/22229

§ 371 Date: Oct. 4, 2012

Related U.S. Application Data
(60) Provisional application No. 61/298,100, filed on Jan. 25, 2010.

(51) Int. Cl. C12N 5/00

(52) U.S. Cl. CPC C12N 5/0018
USPC 435/346; 435/375; 435/358; 435/363

(57) ABSTRACT

The invention relates to compositions, and uses thereof, which are beneficial for eukaryotic cells in culture, and methods for their use in promoting cell growth, viability and recombinant protein expression. The methods disclosed in the present application are useful, for example, for improving cell viability and in accelerating the rate of cell growth of cells grown in culture. In one aspect, the supplements of the invention are useful for improving or enhancing the yield of the recombinant proteins from the cell cultures.

### CROSS-REFERENCE TO RELATED APPLICATIONS

This application claims the benefit of U.S. provisional patent application No. 61/298,100 filed on Jan. 25, 2010, the entire contents of which are incorporated herein by reference.

### TECHNICAL FIELD

The invention relates to compositions, and uses thereof, which are beneficial for eukaryotic cells in culture, and methods for their use in promoting cell growth, viability and recombinant protein expression.

### BACKGROUND

Investigation of biological processes often requires the examination of those processes in cells, tissues and organs that comprise less than the entire organism. For many years these cells, tissues and organs have been separated from the organism and studied independently under conditions that support their survival in an ex vivo or in vitro mode. Typically, the cells, tissues or organs are removed from the organism and are maintained in a culture media that supports the survival and/or biological process being studied. Given the large diversity of cell, tissue and organ types, the formulation of culture medium that support their survival, growth and biological properties outside of the intact organism are not trivial. Many cells and tissues are difficult to maintain in culture for reasons that are not entirely understood. In addition, cells and tissues are often used in processes involved in the manufacturing of recombinant proteins, vaccines, virus stocks and other products in vitro which are key to biomedical research and biologics-based medicaments. Therefore, there is a need to identify conditions and culture media components that support the growth and survival of cells, tissue and organ cultures under in vitro and ex vivo conditions.

Such cell components include for example, albumin, transferrin, glutathione S-transferees, superoxide dismutase, lactoferrin, and growth factors.

Albumin is the most abundant protein found in the plasma. It is produced by the liver in mammals and functions in a variety of capacities. Albumin is a soluble, monomeric protein which comprises about one-half of the blood serum protein. Albumin functions primarily as a carrier protein for steroids, fatty acids, and thyroid hormones and plays a role in stabilizing extracellular fluid volume. Albumin is a globular unglycosylated serum protein of molecular weight 67,000 and contains five or six internal disulphide bonds. Albumin is synthesized as preproalbumin which has an N-terminal peptide that is removed before the nascent protein is released from the rough endoplasmic reticulum. The product, proalbumin, is in turn cleaved in the Golgi vesicles to produce the secreted albumin.

Albumin is essential for maintaining the osmotic pressure needed for proper distribution of body fluids between intravascular compartments and body tissues. It also acts as a plasma carrier by non-specifically binding several hydrophobic steroid hormones and as a transport protein for hemin and fatty acids. Bovine serum albumin (BSA) has long been used as a supplement in cell culture media as it is a component of fetal bovine serum (FBS) which is commonly added to a basal media at 1-20% total volume. BSA is a major component in a number of defined serum free media formulations since it is readily available in bulk, is relatively cheap, and can be purified to homogeneity relatively easily. Representative sources of albumin include for example, plasma derived from bovine, horse, pig and other mammalian species.

With the advent of the large scale production of recombinant proteins, vaccines and other products destined for human clinical use, stricter requirements on the formulations used in the production of those products have been instigated. Because of the threat of animal-derived materials harboring pathogens that may affect the safety of the products, many existing recombinant production processes have been modified such that all materials or culture components used in the entire process are devoid of animal-derived products. That is, the cell culture components cannot have been isolated or purified from whole animal sources. Therefore, the recombinant production of media supplements, as an alternative to the purification of these supplements directly from the whole animal is preferred. Accordingly, human and cell culture components from other species can be manufactured using recombinant means, using defined tissue culture media, and using highly characterized tissue culture cells, which are certified to be free of viruses and toxins.

One method of preparing recombinant protein based cell culture components is to engineer yeast or plants to over express the protein and then to purify the protein. Plant derived recombinant proteins are particularly attractive as a source of cell culture components for recombinant protein production of human proteins that are intended for therapeutic uses since there are no examples of plant viruses that can also infect humans.

There is currently a high demand for recombinant cell culture components to support the recombinant production of human therapeutic proteins, as well as to grow & differentiate stem cells, and with the continued success and huge potential of such products in the market, more effective ways of producing recombinant cell culture components is desirable. In particular, existing processes for the recombinant production of proteins and the growth and differentiation of stem cells are slow, expensive and arduous. In part these processes are limited by fundamental aspects relating to the rate of cell growth and viability of the recombinant host cells or stem cells respectively. Key aspects of these limitations include i) the ability to rapidly isolate and expand single cell clones from complex mixtures of cells, ii) the ability to promote rapid cell growth, particularly at low densities and in serum free media, iii) the ability to sustain cell growth and viability at very high densities in bioreactors, iv) the ability to cryopreserve and thaw cells and cell banks while maintaining high viability, v) the ability to grow and differentiate stem cell cultures effectively. Accordingly there is a need for improved media and culturing conditions that address these needs and enable the improved growth and viability of cells in culture. The supplements and methods of the invention, by improving the viability and rate of cell growth meet these needs and can also result in an improved yield and quality of recombinant product obtained from a mammalian cell culture production process.

### SUMMARY OF INVENTION

The present invention is based in part on the demonstration that plant derived recombinant cell culture component proteins surprisingly enhanced the cell growth and viability when added to mammalian cells grown in culture to a greater extent than standard purified proteins. Such plant derived cell culture components may be used to create supplements that are useful in tissue and cell culture.

The methods and supplements disclosed in the present application are useful, for example, for improving cell viability and in accelerating the rate of cell growth of cells grown in culture. In one aspect, the supplements of the invention are useful for improving or enhancing the yield of the recombinant proteins from the cell cultures. Further improvements provided by the invention are described in detail below.

In one embodiment, the present invention includes a method for enhancing cell growth of a cell in culture comprising the addition of a supplement to the cell culture medium.

In one embodiment, the present invention includes a method for enhancing the productivity of a cell that has been adapted to serum free media comprising the addition of a supplement to the serum free media.

In one embodiment, the present invention includes a method for reducing the accumulation of lactate in a bioreactor comprising the addition of a supplement to cells in culture in the bioreactor.

In one embodiment, the present invention includes a method or reducing the consumption of glucose and other sugars in a bioreactor comprising the addition of a supplement to cells in culture in the bioreactor.

In one embodiment, the present invention includes a method of reducing time required to produce protein from start of culture to harvest in a bioreactor comprising the addition of a supplement to cells in culture in the bioreactor.

In one embodiment, the present invention includes a method for improving the viability of cells in a bioreactor comprising the addition of a supplement to the bioreactor.

In one embodiment, the present invention includes a method for improving the viability of cells grown under serum free conditions comprising the addition of a supplement to the serum free medium.

In one embodiment, the present invention includes a method for improving the viability of cells when plated at low density comprising the addition of a supplement to the cell culture medium.

In one embodiment, the present invention includes a method for improving the viability of cells grown from single cell clones comprising the addition of a supplement to the cell culture medium.

In one embodiment, the present invention includes a method for improving the viability of primary cells grown in culture comprising the addition of a supplement to the culture medium.

In one embodiment, the present invention includes a method for improving the viability of cells after transfection comprising the addition of a supplement to the cell culture medium prior to, during, or immediately after transfection.

In one embodiment, the present invention includes a method for improving the viability of cell after cryopreservation comprising the addition of a supplement to the cell culture medium prior to, during, or immediately after cryopreservation or thawing.

In one embodiment, the present invention includes a method for improving the yield of a recombinant product produced from cells in culture, comprising the addition of a supplement to the culture.

In one embodiment, the present invention includes a method for improving the purification of a recombinant product produced from cells in culture, comprising the addition of a supplement to the culture.

In one embodiment, the present invention includes a method for reducing the proteolysis of a recombinant product produced from cells in culture, comprising the addition of a supplement to the culture.

In one embodiment, the present invention includes a method for improving the bioactivity of a recombinant product produced from cells in culture, comprising the addition of a supplement to the culture.

In one embodiment, the present invention includes a method for improving the stability of a recombinant product produced from cells in culture, comprising the addition of a supplement to the culture.

In one embodiment, the present invention includes a method for improving the assembly of a recombinant product produced from cells in culture, comprising the addition of a supplement to the culture.

In one embodiment, the present invention includes a method for creating a more human pattern of glycosylation of a recombinant product produced from cells in culture, comprising the addition of a supplement to the culture.

In one embodiment, the present invention includes a method for creating a recombinant product produced from cells in culture with less immunogenicity, comprising the addition of a supplement comprising recombinant albumin to the culture.

In one aspect method of the methods of recombinant production, the viability of the cell in culture is increased.

In one aspect of any of these methods the supplement comprises recombinant albumin; wherein said recombinant albumin is produced in a plant; wherein said supplement has less than about 1 EU of endotoxin/mg of albumin, and wherein said albumin comprises less than about 2% aggregated albumin.

In one aspect of any of these methods the cells are primary cells. In one aspect of any of these methods the cells are stem cells. In one aspect of any of these methods the cells are tissue culture cells. In one aspect of any of these methods the cells are blood cells. In one aspect of any of these methods the cells are primary mononuclear cells. In one aspect of any of these methods the cells are CHO cells. In one aspect of any of these methods the cells are hybridoma cells. In one aspect of any of these methods the cells are Vero cells. In one aspect of any of these methods the cells are sorted by flow cytometry. In one aspect of any of these methods the cells are primary cells isolated by gradient centrifugation. In one aspect of any of these methods the cells are B-cells. In one aspect of any of these methods the cells are T-cells. In one aspect of any of these methods the cells are isolated by flow cytometry. In one aspect of any of these methods the cells are isolated by a micro fluidic device.

In one aspect of any of these methods the supplement comprises at least about 0.01% wt/wt of a heat shock protein. In one aspect of this method the heat shock protein is a rice heat shock protein. In one aspect of this method the heat shock protein is selected from the group consisting of Rice HSP70 genes, and rice endosperm lumenal binding protein. In one aspect of this method the heat shock protein is selected from the group consisting of Rice (gblACJ54890.1l), EEC69073/OsI_{—}37938, and AAB63469.

In one aspect of any of these methods the supplement comprises at least about 0.01% wt/wt HSP70. In one aspect of any of these methods the supplement comprises at least about 0.04% wt/wt HSP70. In one aspect of any of these methods the supplement comprises at least about 0.06% wt/wt HSP70. In one aspect of any of these methods the supplement comprises at least about 0.08% wt/wt HSP70. In one aspect of any of these methods the supplement comprises at least about 0.1% wt/wt HSP70.

In one aspect of any of these methods the supplements comprise recombinant albumin which is added to a final concentration of between about 100 mg/L and about 200 mg/L in one aspect of any of these methods the recombinant albumin is added to a final concentration of between about 200 mg/L and about 400 mg/L. In one aspect of any of these methods the recombinant albumin is added to a final concentration of between about 400 mg/L and about 600 mg/L. In one aspect of any of these methods the recombinant albumin is added to a final concentration of between about 600 mg/L and about 800 mg/L. In one aspect of any of these methods the recombinant albumin is added to a final concentration of between about 800 mg/L and 1000 mg/L. In one aspect of any of these methods the recombinant albumin is added to a final concentration of between about 1000 mg/L and about 2000 mg/L. In one aspect of any of these methods the recombinant albumin is added to a final concentration of between about 2000 mg/L and 5000 mg/L. In one aspect of any of these methods the recombinant albumin is added to a final concentration of between about 5000 mg/L and about 10000 mg/L. In one aspect of any of these methods the recombinant albumin is added to a final concentration of between about 10000 mg/L and about 20000 mg/L.

In one aspect of any of these methods the improvement in cell viability is greater than 10% compared to cell viability of cells grown under identical conditions but without said supplement. In one aspect of any of these methods the improvement in cell viability is greater than 15% compared to cell viability of cells grown under identical conditions but without said supplement. In one aspect of any of these methods the improvement in cell viability is greater than 20% compared to cell viability of cells grown under identical conditions but without said supplement. In one aspect of any of these methods the improvement in cell viability is greater than 25% compared to cell viability of cells grown under identical conditions but without said supplement. In one aspect of any of these methods the improvement in cell viability is greater than 30% compared to cell viability of cell grown under identical conditions but without said supplement. In one aspect of any of these methods the improvement in cell viability is greater than 40% compared to cell viability of cell grown under identical conditions but without said supplement. In one aspect of any of these methods the improvement in cell viability is greater than 50% compared to cell viability of cell grown under identical conditions but without said supplement. In one aspect of any of these methods the improvement in cell viability is greater than 60% compared to cell viability of cell grown under identical conditions but without said supplement. In one aspect of any of these methods the improvement in cell viability is greater than 70% compared to cell viability of cell grown under identical conditions but without said supplement. In one aspect of any of these methods the improvement in cell viability is greater than 80% compared to cell viability of cell grown under identical conditions but without said supplement. In one aspect of any of these methods the improvement in cell viability is greater than 90% compared to cell viability of cell grown under identical conditions but without said supplement. In one aspect of any of these methods the improvement in cell viability is greater than 100% compared to cell viability of cell grown under identical conditions but without said supplement.

### BRIEF DESCRIPTION OF FIGURES

A better understanding of the features and advantages of the present invention can be obtained by reference to the following detailed description that sets forth illustrative embodiments, in which the principles of the invention are utilized, and the accompanying drawings of which:

FIG. 1 Shows a comparison by HPLC size exclusion chromatography of recombinant albumin produced from rice compared to other sources of albumin and methods of purification.

FIG. 1A shows the chromatogram for a serum derived (non-recombinant albumin). FIG. 1B shows the chromatogram for a rice recombinant albumin (Cellastim P0107) made using the “old process” B000 for purification. FIG. 1C shows the chromatogram for a rice recombinant albumin (Cellastim P0171) made using the “new process” B0000C for purification. FIG. 1D shows an overlay of the chromatograms for the serum derived albumin (1A; dotted line) and Cellastim prepared using the new process ((1C; solid line). FIG. 1E shows an overlay of the chromatograms for Cellastim prepared using the old process B000 (Cellastim P0107)(1B; dotted line) and Cellastim prepared using the new process B0000C (Cellastim P0171)(1C; solid line).

FIG. 2 Shows a comparison by SDS PAGE analysis of recombinant albumin produced from rice compared to other sources of albumin and methods of purification. FIG. 2A shows a comparison of Cellastim P0171 and Cellprime albumin (Millipore/Novozymes). Lane 1 is the molecular weight marker. Lane 4 is the Cellastim albumin (10 μg) and Lane 7 is the Cellprime albumin (10 μg). FIG. 2B shows a comparison by SDS PAGE analysis of three Cellastim lots from the previous process (B000) (Lane 2, 3, and 4), and the new Cellastim Process (B0000C) (Lane 6, 7, and 8). The six samples were loaded at 20 μg per lane.

FIG. 3: Shows a comparison of the effects of yeast recombinant (Cellprime), human derived, (Seracare) and plant recombinant albumin (Cellastim P0171) with respect to cell growth and viability. (FIG. 3A). FIG. 3B shows a comparison of the endotoxin levels in batches of albumin produced using the old (B000) and new processes (B0000C) for recombinant albumin production. FIG. 3C shows a comparison of cell growth and viability of cells grown in the presence of the Cellastim produced using the old (B000) and new processes (B0000C) for recombinant albumin production.

FIG. 4: Shows a western blot using an anti-heat shock protein antibody to show the heat shock protein content of different fractions obtained from recombinant albumin after ATP affinity chromatography. (See Example 3)

FIG. 5: Shows a comparison of the cell growth and viability effect of Cellastim recombinant albumin after passing the albumin produced using the new process over an ATP affinity column to remove heat shock proteins. (See text for details).

FIG. 6A. Shows a Growth profile of CHO-K1 in unsupplemented and supplemented medium in shake flasks. FIG. 6B Shows the percentage of viable cells of CHO-K1 in unsupplemented and supplemented medium.

FIG. 7A: Shows the specific net growth rate of CHO K1 cells grown in supplemented and unsupplemented (control) medium in shake flasks. FIG. 7B shows the specific net death rate of CHO K1 cells grown in supplemented and unsupplemented (control) medium in shake flasks.

FIG. 8A Shows the viability cell density of in unsupplemented and supplemented medium (nutrient feed added on day 4). FIG. 8B. Shows the percentage of viable cells of CHO-K1 in unsupplemented and supplemented medium (nutrient feed added on day 4).

FIG. 9A: Shows the specific net growth rate of CHO K1 cells grown in supplemented and unsupplemented (control) medium in shake flasks (boosted with nutrient feed on day 4). FIG. 9B. Shows the specific net death rate of CHO K1 cells grown in supplemented and unsupplemented (control) medium in shake flasks (Boosted with nutrient feed on day 4). FIG. 9C. Shows the increased concentration of antibody in medium with supplements in shake flasks.

FIG. 10A shows the Growth profile of CHO K1 in bioreactors after adverse event on loading. Two bioreactors were run for the 250 mg/L Cellastim condition. FIG. 10B. Shows the percentage of viable cells of CHO K1 in bioreactors after adverse event on loading. Two bioreactors were run for the 250 mg/L Cellastim conditions.

FIG. 11A. Shows the growth profile of CHO K1 in bioreactors in supplemented and unsupplemented control medium (with nutrient boost on days 3 and 7). The viable cell density over time is shown. FIG. 11B. Shows the specific growth rate of CHO K1 in bioreactors in supplemented and unsupplemented control medium (with nutrient feed on days 3 and 7). Viable cell density over time is shown.

FIG. 12A. Shows the percentage of viable cells of CHO K1 in bioreactors after adverse in unsupplemented and supplemented medium (with nutrient feed on day 3 and 7). FIG. 12B. Shows the specific net death rate of CHO K1 cells grown in supplemented and unsupplemented (control) medium in bioreactors (Boosted with nutrient feed on day 3 and 7).

FIG. 13A. Shows the pH trends for CHO K1 grown in supplemented and unsupplemented medium in bioreactors. FIG. 13B. Shows the osmolality trends for CHO K1 grown in supplemented and unsupplemented medium in bioreactors.

FIG. 14A. Shows the glucose trends for CHO K1 grown in supplemented and unsupplemented medium in bioreactors (with nutrient feed on day 3 and 7). FIG. 14B. Shows the lactate trends for CHO K1 grown in supplemented and unsupplemented medium in bioreactors (with nutrient feed on day 3 and 7).

FIG. 15A. Shows the specific glucose consumption of CHO K1 cells grown in supplemented and unsupplemented (control) medium in bioreactors (Boosted with nutrient feed on day 3 and 7). FIG. 15B. Shows the specific lactate production of CHO K1 cells grown in supplemented and unsupplemented (control) medium in bioreactors (Boosted with nutrient feed on day 3 and 7).

FIG. 16A. Shows the concentration of product produced by CHO K1 in supplemented and unsupplemented medium in bioreactors (with nutrient feed on day 3 and day 7). FIG. 16B Shows the specific productivity of CHO K1 in supplemented and unsupplemented medium in bioreactors (with nutrient feed on day 3 and day 7).

FIG. 17A Shows the schematic representation of methods used in the purification of antibody by protein A chromatography. FIG. 17B: Shows the absorbance chromatogram showing equilibration, loading, washing, and eluded fractions. Note that there is one strong peak of protein present in the eluded fraction representing purified antibody.

FIG. 18 Shows the SDS-PAGE with Coomassie blue staining showing the purification of antibody and the successful removal of the media supplements by protein A chromatography.

FIG. 19. Shows the SDS-PAGE with silver staining showing the purification of antibody and the successful removal of the media supplements by protein A chromatography.

### DETAILED DESCRIPTION OF INVENTION

### Definitions

In order that the present disclosure may be more readily understood, certain terms are first defined. Additional definitions are set forth throughout the detailed description.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. As used herein, the term “increase” or the related term “increased” refers to a statistically significant increase. For the avoidance of doubt, the terms generally refer to at least a 10% increase in a given parameter, and can encompass at least 20%, 50%, 75%, 100%, 150% or more.

The term “antigen-binding fragment” refers to a polypeptide portion of an immunoglobulin or antibody that binds antigen or competes with intact antibody (i.e., with the intact antibody from which they were derived) for antigen binding (i.e., specific binding). Binding fragments can be produced by recombinant DNA techniques, or by enzymatic or chemical cleavage of intact immunoglobulins. Binding fragments include Fab, Fab′, F(ab′)₂, Fabc, Fv, single chains, and single-chain antibodies.

The term “apoptosis” (“normal” or “programmed” cell death) refers to the physiological process by which unwanted or useless cells are eliminated during development and other normal biological processes. Apoptosis is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine dependent tissue atrophy. Apoptosis may also be triggered in cells grown under tissue culture conditions in response to stress. Cells undergoing apoptosis show characteristic morphological and biochemical features, which can be readily measured and quantified. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis”.

As used herein, the terms “cell,” “cells,” “cell line,” “host cell,” and “host cells,” are used interchangeably and, encompass plant, and animal cells and include invertebrate, non-mammalian vertebrate and mammalian cells. All such designations include cell populations and progeny. Thus, the terms “transformants” and “transfectants” include the primary subject cell and cell lines derived therefrom without regard for the number of transfers. Exemplary non-mammalian vertebrate cells include, for example, avian cells, reptilian cells and amphibian cells. Exemplary invertebrate cells include, but are not limited to, insect cells such as, for example, caterpillar (Spodoptera frugiperda) cells, mosquito (Aedes aegypti) cells, fruitfly (Drosophila melanogaster) cells, Schneider cells, and Bombyx mori cells. See, e.g., Luckow et al., Bio/Technology 6:47-55 (1988). The cells may be differentiated, partially differentiated or undifferentiated, e.g. stem cells, including embryonic stem cells and pluripotent stem cells. Additionally tissue samples derived from organs or organ systems may be used according to the invention. Exemplary mammalian cells include, for example, cells derived from human, non-human primate, cat, dog, sheep, goat, cow, horse, pig, rabbit, rodents including mouse, hamster, rat and guinea pig and any derivatives and progenies thereof.

The terms “cell culture,” or “tissue culture” refer to cells grown in suspension or grown adhered to a variety of surfaces or substrates in vessels such as roller bottles, tissue culture flasks, dishes, multi-well plates and the like. Large scale approaches, such as bioreactors, including adherent cells growing attached to microcarriers in stirred fermentors, are also encompassed by the term “cell culture.” Moreover, it is possible not only to culture contact-dependent cells, but also to use suspension culture techniques in the methods of the claimed invention. Exemplary microcarriers include, for example, dextran, collagen, plastic, gelatin and cellulose and others as described in Butler, Spier & Griffiths, Animal cell Biotechnology 3:283-303 (1988). Porous carriers, such as, for example, Cytoline™ or Cytopore™, as well as dextran-based carriers, such as DEAE-dextran (Cytodex 1™ quaternary amine-coated dextran (Cytodex™) or gelatin-based carriers, such as gelatin-coated dextran (Cytodex 3™) may also be used. Cell culture procedures for both large and small-scale production of proteins are encompassed by the present invention. Procedures including, but not limited to, a fluidized bed bioreactor, hollow fiber bioreactor, roller bottle culture, or stirred tank bioreactor system may be used, with or without microcarriers, and operated alternatively in a batch, fed-batch, or perfusion mode.

The terms “cell culture medium,” “cell culture media,” and “culture medium” refer to the solutions used for growing, storing, handling and maintaining cells and cell lines. Such solutions generally include various factors necessary for cell attachment, growth, and maintenance of the cellular environment. For example, a typical solution may include a basal media formulation, various supplements depending on the cell type and, occasionally, antibiotics. In some embodiments, a solution may include at least one component from one or more of the following categories: 1) an energy source, usually in the form of a carbohydrate such as glucose; 2) all essential amino acids, and usually the basic set of twenty amino acids plus cystine; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The solution may optionally be supplemented with one or more components from any of the following categories: 1) hormones and other growth factors as, for example, insulin, transferrin, and epidermal growth factor; 2) salts and buffers as, for example, calcium, magnesium, phosphate, Tris, HEPES, and sodium bicarbonate; 3) nucleosides and bases such as, for example, adenosine and thymidine, hypoxanthine; and 4) protein and tissue hydrolysates. In general, any suitable cell culture medium may be used. The medium may be comprised of serum, e.g. fetal bovine serum, calf serum or the like. Alternatively, the medium may be serum free, animal free, or protein free.

The term “cell lineage” when referring to a stem cell culture refers to all of the stages of the development of a cell type, from the earliest precursor cell to a completely mature cell (i.e. a specialized cell).

The terms “cell viability” or “viability” refers to relative amounts of living and dead cells, present with a population of cells at any given time. Cell viability may be determined by measuring the relative numbers of living and dead cells in any given sample of the population. Cell viability may also be estimated by measuring the rate of cell proliferation of the entire population which represents the overall balance of the rates of cell growth and cell death. Rates of cell growth may also be directly measured, by counting the number of cells, and by using any number of commercially available cell proliferation assays which directly scores the rate of cell growth.

“Conditioned medium” refers to a cell culture medium that is obtained from a culture of a feeder cell on which stem cells can be cultured and maintained in a pluripotent state. The feeder cell depletes the conditioned medium of some components, but also enriches the medium with cell-derived material, probably including small amounts of growth factors. The term “feeder cell factor” as used herein means the cell-derived material that is released into the conditioned medium by the feeder cell. The cell factor that is released into the cell culture medium is useful in enhancing the growth of stem cells, or in the maintenance of the embryonic stem cell in a pluripotent state. The feeder cell factor can be identified and purified using techniques that are known to one skilled in the art, and are described herein.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

The term “cytotoxicity” refers to the cell killing property of a chemical compound (such as a chemical or protein contaminant, detergent, or toxin). In contrast to necrosis and apoptosis, the term cytotoxicity need not necessarily indicate a specific cellular death mechanism.

As used herein, the term “decrease” or the related terms “decreased,” “reduce” or “reduced” refers to a statistically significant decrease. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero).

As used herein, the terms “develop”, “differentiate” and “mature”, as used to describe a stem cell, refer to the progression of a cell from the stage of having the potential to differentiate into at least two different cellular lineages to becoming a specialized and terminally differentiated cell. Such terms can be used interchangeably for the purposes of the present application.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are DNA regulatory sequences, such as promoters, enhancers, polyadenylation signals, terminators, internal ribosome entry sites (IRES) and the like, that provide for the expression of a coding sequence in a host cell. Exemplary expression control sequences are described in Goeddel; Gene Expression Technology: Methods in Enzymology 185, Academic Press, San Diego, Calif. (1990).

The term “feeder cell” refers to a culture of cells that grows in vitro and secretes at least one factor into the culture medium, and that can be used to support the growth of another cell of interest in culture. As used herein, a “feeder cell layer” can be used interchangeably with the term “feeder cell.” A feeder cell can comprise a monolayer, where the feeder cells cover the surface of the culture dish with a complete layer before growing on top of each other, or can comprise clusters of cells.

The term “growth phase” of the cell culture refers to the period of exponential cell growth (the log phase) where cells are dividing at a constant rate. During this phase, cells are cultured for a period of time, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. During the growth phase, cells are cultured in nutrient medium containing the necessary additives usually at about 30-40° C., generally about 37° C., in a humidified, controlled atmosphere, such that optimal growth is achieved for the particular cell line, for instance a mammalian cell.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., Cell, 50:667, 1987). Such proteins (and their encoding nucleic acids) have sequence homology, as reflected by their sequence similarity, whether in terms of percent identity or by the presence of specific residues or motifs and conserved positions.

The term growth factor refers to Amphiregulin, Angiopoietin, Betacellulin, (Bone Morphogenic protein-13, Bone Morphogenic protein-14, Bone Morphogenic protein-2, Human BMP-3, Bone Morphogenic protein-4, Human BMP-5, Bone Morphogenic protein-6, Bone Morphogenic protein-7, Human CD135 Ligand/Flt-3 Ligand, Human Granulocyte Colony Stimulating Factor (G-CSF), Human Granulocyte Macrophage Colony Stimulating Factor (GM-CSF), Human Macrophage Colony Stimulating Factor (M-CSF), Human Cripto-1, Human CTGF (Connective tissue growth factor), Human EGF (Epidermal Growth Factor), Human EG-VEGF (Endocrine-Gland-Derived Vascular Endothelial Growth Factor), Human Erythropoietin (EPO), Human FGF (Fibroblast Growth Factors 1-23), Human GDF-11, Human GDF-15, Human GDF-8, Human Growth Hormone Releasing Factor (GHRF, GRF, GHRH, Growth Hormone Releasing Hormone), Human Heparin Binding Epidermal Growth Factor (HB-EGF), Human Hepatocyte Growth Factor (HGF), Human Heregulin beta 1, Human insulin, Human IGF-1 (Insulin-like Growth Factor-1), Human IGF-2 (Insulin-like Growth Factor-2), Human IGFBP-1 (Insulin-like Growth Factor Binding Protein 1), Human IGFBP-3 (Insulin-like Growth Factor Binding Protein 3), intestinal trefoil factor (ITF), Human keratinocyte growth factors 1 & 2, Human Leukemia Inhibitory Factor (LIF), Human MSP, Human Myostatin, Human Myostatin, pro (propeptide), Human NRG1, Human NGF, Human Oncostatin M, Human Osteoblast Specific Factor 1 (OSF-1, Pleiotrophin), Human PD-ECGF (Platelet-derived endothelial cell growth factor), Human PDGF, Human PDGF, Human Placental Growth Factor 1 (PLGF1), Human Placental Growth Factor 2 (PLGF2), Human SCGF-a (Stem Cell Growth Factor-alpha), Human SCGF-b (Stem Cell Growth Factor-beta), Human Stem Cell Factor (SCF)/CD117 Ligand, Human Thrombopoietin (TPO, THPO), Human Transforming Growth Factor, Human TGF-alpha (Transforming Growth Factor-alpha, TGFa), Human TGF-beta 1 (Transforming Growth Factor-beta1, TGFb), Human TGF-beta 1.2 (Transforming Growth Factor-beta1, TGFb), Human TGF-beta 2 (Transforming Growth Factor-beta2, TGFb), Human TGF-beta 3 (Transforming Growth Factor-beta3, TGFb), Human VEGF (Vascular Endothelial Growth Factor), Human VEGF-121, Human VEGF-165, Human VEGF-A

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm can also be used to determine identity.

The terms “immunoglobulin” or “antibody” (used interchangeably herein) refers to a protein typically having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by beta-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains). Immunoglobulins or antibodies may be monoclonal or polyclonal and may exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments.

The term “isolated,” when used to describe the cell culture components, or heat shock proteins disclosed herein, means protein that has been identified and separated and/or recovered from a component of its natural environment. Contaminant components of its natural environment are materials that would typically interfere with research, diagnostic or therapeutic uses for the protein, and may include enzymes, hormones, and other proteinaceous or non-proteinaceous solutes. In some embodiments, the protein will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated protein will be prepared by at least one purification step.

“Markers” as used herein, are nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

Cells expressing “markers of pancreatic endocrine lineage” refer to cells with positive gene expression for the transcription factor PDX-1 and at least one of the following transcription factors: NGN-3, NRx2.2, NRx6.1, NeuroD, Is1-1, HNF-3 beta, MAFA, Pax4, and Pax6. Cells expressing markers characteristic of the pancreatic cell lineage include pancreatic β cells.

Cells expressing “markers characteristic of endoderm lineage” as used herein refer to cells expressing at least one of the following markers: SOX-17, GATA-4, HNF-3 beta, GSC, Cer1, Nodal, FGF8, Brachyury, Mix-like homeobox protein, FGF4 CD48, eomesodermin (EOMES), DKK4, FGF17, GATA-6, CXCR4, C-Kit, CD99, or OTX2. Cells expressing markers characteristic of the definitive endoderm lineage include primitive streak precursor cells, primitive streak cells, mesendoderm cells and definitive endoderm cells.

Cells expressing pluripotency markers derived by the methods of the present invention express at least one of the following pluripotency markers selected from the group consisting of: ABCG2, cripto, FoxD3, Connexin43, Connexin45, Oct4, SOX-2, Nanog, hTERT, UTF-1, ZFP42, SSEA-3, SSEA-4, Tral-60, and Tral-81.

Cells expressing “markers characteristic of mesoderm lineage” as used herein refers to a cell expressing at least one of the following markers: CD48, eomesodermin (EOMES), SOX-17, DKK4, HNF-3 beta, GSC, FGF17, GATA-6.

Cells expressing “markers characteristics of ectoderm lineage” as used herein refers to a cell expressing at least one of the following markers: BMP-4. Noggin, Chordin, Otx2, Fox J3, Nestin, p63/TP73L, beta-III Tubulin.

The terms “operably linked” and “operatively linked,” as used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more nucleotide sequences chosen from: (a) a nucleotide sequence capable of increasing translation; (b) a nucleotide sequence capable of increasing secretion of the recombinant protein outside a cell; and (c) a nucleotide sequence capable of increasing the mRNA stability, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The terms “polynucleotide” and “nucleic acid molecule,” used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, “DNA” or “nucleotide sequence” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The term “pluripotent stem cell” encompasses stem cells obtained from embryos, fetuses or adult tissues. In one preferred embodiment, the pluripotent stem cell is an embryonic stem cell. In another embodiment the pluripotent stem cell is a fetal stem cell, such as a primordial germ cell. In another embodiment the pluripotent stem cell is an adult stem cell.

As used herein, the term “pluripotent” refers to a cell capable of at least developing into one of ectodermal, endodermal and mesodermal cells. As used herein the term “pluripotent” includes cells that are totipotent and multipotent. As used herein, the term “totipotent cell” refers to a cell capable of developing into all lineages of cells. The term “multipotent” refers to a cell that is not terminally differentiated.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter, the rice endosperm specific glutelin (Gt1) promoter, CaMV35S viral promoter. Inducible promoters include the Tet system, (U.S. Pat. Nos. 5,464,758 and 5,814,618), the Ecdysone inducible system (No et al., Proc. Natl. Acad. Sci. (1996) 93 (8): 3346-3351; the T-REₓ™ system (Invitrogen Carlsbad, Calif.), LacSwitch® (Stratagene, (San Diego, Calif.) and the Cre-ER™ tamoxifen inducible recombinase system (Indra et al. Nuc. Acid. Res. (1999) 27 (22): 4324-4327; Nuc. Acid. Res. (2000) 28 (23): e99; U.S. Pat. No. 7,112,715; and Kramer & Fussenegger, Methods Mol. Biol. (2005) 308: 123-144) or any promoter known in the art suitable for expression in the desired cells.

The term “protein of interest” refers to any protein which may be useful for research, diagnostic or therapeutic purposes. The protein of interest may comprise a mammalian protein or non-mammalian protein, and may optionally comprise a receptor or a ligand. Exemplary proteins of interest include, but are not limited to, molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; members of the TNF and TNF receptor (TNFR) family, like tumor necrosis factor-alpha and -beta, CD40 ligand, Apo-2 ligand/TRAIL, DR4, DR5, DcR1, DcR2, DcR3, OPG, Fas ligand; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TG-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; thrombopoietin (TPO); interleukins (ILs), e.g., IL-1 to IL-10; superoxide dismutase; T-cell receptors; surface membrane proteins; decay accelerating factor; viral antigen such as, for example, a portion of the AIDS envelope, gp120; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and variants and/or fragments of any of the above-listed polypeptides; as well as antibodies against various protein antigens like CD proteins such as CD3, CD4, CD8, CD19, CD20 and CD34; members of the ErbB receptor family such as the EGF receptor, HER2, HER3 or HER4 receptor; cell adhesion molecules such as LFA-1, Mac1, p150.95, VLA-4, ICAM-1, VCAM and αv/β3 integrin including either α or β subunits thereof (e.g. anti-CD11a, anti-CD18 or anti-CD11b antibodies); growth factors such as VEGF; IgE; blood group antigens; flk2/flt3 receptor; obesity (OB) receptor; mpl receptor; CTLA-4; protein C; an Apo-2L receptor such as Apo-2 (DR5), DR4, DcR1, DcR2, DcR3; and variants and/or fragments of the above-identified antibodies etc. In one embodiment of the invention, a protein of interest will comprise a protein which itself is capable of inducing apoptosis in mammalian or non-mammalian cells in vitro or in vivo, such as Apo-2 ligand/TRAIL, Fas ligand, or TNF-alpha.

The term “production phase” of the cell culture refers to the period of time during which cell growth has reached a plateau. During the production phase, logarithmic cell growth has ended and protein production is primary. During this period of time the medium is generally supplemented to support continued protein production and to achieve the desired protein product.

The term “recombinant protein” or “recombinant polypeptide” refers to an exogenous, i.e., heterologous or foreign polypeptide, to the cells producing the polypeptide.

The term “stress” in the context of apoptosis or cell culture refers to non-optimal conditions for tissue culture including any combination of the following; the presence of toxins, nutrient or growth factor depletion or withdrawal, hypoxia, thermal stress (temperature is too high or too low compared to the preferred range), loss of cell-cell contacts, viral infection, osmotic stress (osmolality is too high or too low compared to the preferred range), oxidative stress, cell density (cell density is too high or too low compared to the preferred range), and pH stress (pH is too high or too low compared to the preferred range).

The term “transformation” refers to the transfer of one or more nucleic acid molecules into a host cell or organism. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction or infection with viruses or other infectious agents. “Transformed”, “transduced”, “transgenic”, and “recombinant” refer to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells have been through the transformation process and contain foreign nucleic acid. The term “untransformed” refers to cells that have not been through the transformation process.

The term “transition phase” of the cell culture refers to the period of time during which culture conditions for the production phase are engaged. During the transition phase environmental factors such as pH, ion concentration, and temperature may shift from growth conditions to production conditions.

The term “sequence similarity” refers to the degree of identity or correspondence between nucleic acid or amino acid sequences that may or may not share a common evolutionary origin (see Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3, 4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The publications discussed above are provided solely for their disclosure before the filing date of the present application. Nothing herein is to be construed as an admission that the invention is not entitled to antedate such disclosure by virtue of prior invention.

### I Methods for Using the Supplements of the Invention

The claimed supplements are useful in a wide range of applications for tissue and cell culture and recombinant protein production where they provide for significant improvements in preventing apoptosis and improving cell viability during tissue culture, and in particular in response to stress.

Apoptosis involves a series of biochemical events leading to a characteristic cell morphology and death. These changes include, changes to the cell membrane such as loss of membrane asymmetry and attachment, cellular blebbing cell shrinkage, nuclear fragmentation, chromatin condensation, and chromosomal DNA fragmentation.

The process of apoptosis is controlled by a diverse range of cell signals, which may originate either extracellularly (extrinsic inducers) or intracellularly (intrinsic inducers). Extracellular signals may include toxins, hormones, growth factors, nitric oxide, cytokines, which may be present to different degrees in tissue culture media. These signals may positively (i.e., trigger) or negatively (i.e., repress, inhibit, or dampen) affect apoptosis, and thus influence overall cell viability. A number of intracellular components, including ATP content, calcium level, and a number of apoptotic and anti-apoptotic genes also help regulate apoptosis. A cell may initiate intracellular apoptotic signaling in response to a stress, which may bring about cell suicide. Stress inducing agents encountered during tissue culture include for example toxins, associated with tissue culture components such as endotoxins, and heavy metals that leach from plastic ware, transfection reagents (e.g. Lipofectamine and similar lipid based transfection reagents), viral transformation, nutrient and growth factor deprivation, associated with serum free culture, or cell differentiation protocols hypoxia and oxidative stress associated with high density culture in a bioreactor and increased intracellular calcium concentration, for example, by damage to the membrane caused by detergents and electroporation.

Before the actual process of cell death occurs, the apoptotic signals must overcome regulatory proteins which act as gatekeepers overseeing the activation of the apoptosis pathway. In vivo, this step allows the process to be stopped, should the cell no longer need to die. Several proteins are involved at this step, though two main mechanisms of regulation have been identified and include those associated with mitochondria functionality, and those directly involved in transducing the signal via adaptor proteins to the apoptotic mechanisms.

Cells grown under cell culture conditions may experience cellular stresses associated with routine tissue culture procedures, as described above which may trigger apoptotic signals and increase the susceptibility of the cells to apoptosis. For example, nutrient deprivation associated with serum free culture, oxidative stress associated with high density growth in a bioreactor, the use of cytotoxic compounds associated with DNA transfection reagents, and thermal stresses associated with cryopreservation, may predispose the cell to enter apoptosis. By enhancing the ability of a cell to survive such signals it is possible to improve cell viability during these procedures, by preventing the cells commitment to cell death, thereby improving the success and utility of these approaches.

Recently a number of genes in eukaryotic cells have been identified which inhibit the onset or reduce the effects of apoptosis. Some of these genes inhibit caspase dependent apoptotic pathways in the cell, and in fact transfecting cells with anti-apoptotic genes may be useful in prolonging the life and productivity of transfected cells grown under biologically demanding conditions. (U.S. Pat. Nos. 6,586,206; 7,531,327; US Patent Application US 2009/0170165; US2009/0181426).

Additionally the addition of exogenous heat shock proteins has in some cases been shown to improve the survival of cells in culture under a variety of conditions. (Novoselova et al., J. Neurochem. 94 597-606 (2005); Tidwell et al., Cell Stress & Chap 9 (1) 88-90 (2004); Guzhova et al., Cell Stress & Chap. 3 (1) 67-77 (1998); Hounenou et al., Cell Stress & Chap 1 (3) 161-166 (1996); Johnson et al., In vitro Cell. Dev. Biol., 29A 807-812 (1993).

The present invention is based in part on the demonstration that plant derived recombinant cell culture component proteins surprisingly enhanced the cell growth and viability when added to mammalian cells grown in culture. Specifically, such supplements result in improved culture viability, extended cell survival, improved rates of cell growth and improved yields of recombinant proteins produced from tissue culture bioreactors. Because the supplements show unexpectedly improved activity and stability they offer significant improvements compared to the use of standard recombinant or purified proteins.

The methods disclosed in the present application are useful, for example, for improving cell viability and in accelerating the rate of cell growth of cells grown in culture. In one aspect, the supplements of the invention are useful for improving or enhancing the yield of the recombinant proteins from the cell cultures. Further improvements provided by the invention are described in detail below.

In one embodiment, the present invention includes a method for enhancing cell growth of a cell in culture comprising the addition of a supplement to the cell culture medium.

In one embodiment, the present invention includes a method for enhancing the productivity of a cell that has been adapted to serum free media comprising the addition of a supplement to the serum free media.

In one embodiment, the present invention includes a method for reducing the accumulation of lactate in a bioreactor comprising the addition of a supplement to cells in culture in the bioreactor.

In one embodiment, the present invention includes a method or reducing the consumption of glucose and other sugars in a bioreactor comprising the addition of a supplement to cells in culture in the bioreactor.

In one embodiment, the present invention includes a method of reducing time required to produce protein from start of culture to harvest in a bioreactor comprising the addition of a supplement to cells in culture in the bioreactor.

In one embodiment, the present invention includes a method for improving the viability of cells in a bioreactor comprising the addition of a supplement to the bioreactor.

In one embodiment, the present invention includes a method for improving the viability of cells grown under serum free conditions comprising the addition of a supplement to the serum free medium.

In one embodiment, the present invention includes a method for improving the viability of cells when plated at low density comprising the addition of a supplement to the cell culture medium.

In one embodiment, the present invention includes a method for improving the viability of cells grown from single cell clones comprising the addition of a supplement to the cell culture medium.

In one embodiment, the present invention includes a method for improving the viability of primary cells grown in culture comprising the addition of a supplement to the culture medium.

In one embodiment, the present invention includes a method for improving the viability of cells after transfection comprising the addition of a supplement to the cell culture medium prior to, during, or immediately after transfection.

In one embodiment, the present invention includes a method for improving the viability of cell after cryopreservation comprising the addition of a supplement to the cell culture medium prior to, during, or immediately after cryopreservation or thawing.

In one embodiment, the present invention includes a method for improving the rate of cell growth or viability of stem cells grown in culture comprising the addition of a supplement of the present invention to the cell culture media.

In one embodiment, the present invention includes a method for improving the yield of a recombinant product produced from cells in culture comprising the addition of a supplement of the present invention to the cell culture media during one or more of the growth phase, transition phase, or production phase of the culture.

In one embodiment, the present invention includes a method for improving the purification of a recombinant product produced from cells in culture, comprising the addition of a supplement to the culture media during one or more of the growth phase, transition phase, or production phase of the culture.

In one embodiment, the present invention includes a method for reducing the proteolysis of a recombinant product produced from cells in culture, comprising the addition of a supplement to the culture media during one or more of the growth phase, transition phase, or production phase of the culture.

In one embodiment, the present invention includes a method for improving the bioactivity of a recombinant product produced from cells in culture, comprising the addition of a supplement to the culture media during one or more of the growth phase, transition phase, or production phase of the culture.

In one embodiment, the present invention includes a method for improving the stability of a recombinant product produced from cells in culture, comprising the addition of a supplement to the culture media during one or more of the growth phase, transition phase, or production phase of the culture.

In one embodiment, the present invention includes a method for improving the assembly of a recombinant product produced from cells in culture, comprising the addition of a supplement to the culture media during one or more of the growth phase, transition phase, or production phase of the culture.

In one embodiment, the present invention includes a method for creating a more human pattern of glycosylation of a recombinant product produced from cells in culture, comprising the addition of a supplement to the culture media during one or more of the growth phase, transition phase, or production phase of the culture.

In one embodiment, the present invention includes a method for creating a recombinant product produced from cells in culture with less immunogenicity, comprising the addition of a supplement comprising recombinant albumin to the culture media during one or more of the growth phase, transition phase, or production phase of the culture.

In any of these methods, the supplements of the invention, by increasing host cell viability in culture (and during fermentation), provide for a simple and cost effective method to increase the yield, and or purity, bioactivity, stability and assembly of functional recombinant protein. Additionally, the supplements of the invention, by decreasing or inhibiting apoptosis in the cell culture, can decrease the number or presence of adverse proteases in the culture media and protect the expressed protein of interest against proteolytic degradation, thereby increasing the quality of the protein of interest produced, as evidenced by increased amounts of active protein, and increased yields of intact protein. Additionally Applicants have found that the supplements of the invention may protect the cells against potential adverse effects of agents like detergents, heavy metals and endotoxin contaminates present in the culture components, or protect the cells from toxic reagents introduced to the cells during transfection or cryopreservation.

In any of the claimed methods, the supplements of the invention can be added directly, or admixed, to the culture media at any convenient time, for example when changing the media, passaging the cells, or when plating out the cells at low density. Optionally, the supplement is added to the culture media at the beginning (at the time of initiating, day 0) of the cell culturing process. In one aspect the supplements of the invention may be added before an anticipated stressful event, for example before cryopreservation, transfection or serum withdrawal, etc.

In another aspect, the supplement is added to the culture media during the culturing of the cells prior to the point when induction of typically apoptosis occurs. For example, during a large scale cell culture, induction of apoptosis can be observed on about day 3 or day 4 of the culture, and therefore, the supplement will preferably be added prior to day 3 or day 4. Optionally, a desired quantity of the supplement is added throughout, or for the duration of, the cell culture, for instance, on a daily basis for the entire fermentation. As an example, for a 5 day culture, the supplement could be added at day 0, and every 24 hours thereafter until the culture is terminated.

Accordingly in one embodiment, the invention provides a method of improving the yield and quality of a recombinant protein produced in a bioreactor by adding a supplement of the invention to the bioreactor. In one embodiment, the bioreactor comprises bacterial cells. In another aspect the bioreactor comprises yeast cells. In another aspect the bioreactor comprises plant cells. In another aspect the bioreactor comprises mammalian cells.

In another embodiment, the invention provides a method of improving the yield and quality of a recombinant protein produced in bacterial cells, by adding the supplement of the invention to the cell culture. In another embodiment, the invention provides a method of improving the yield and quality of a recombinant protein produced in yeast cells by adding the supplement of the invention to the cell culture. In another embodiment, the invention provides a method of improving the yield and quality of a recombinant protein produced in a plant cells by adding the supplement of the invention to the cell culture. In another embodiment, the invention provides a method of improving the yield and quality of a recombinant protein produced in insect cells by adding the supplement of the invention to the cell culture. In another embodiment, the invention provides a method of improving the yield and quality of a recombinant protein produced in mammalian cells by adding the supplement of the invention to the cell culture.

In another embodiment, the invention provides a method to increase the yield of the production phase of a cell culture system and thereby increase the productivity of a bioreactor by adding the supplement of the invention to the cell culture system prior to, or during the production phase of the cell culture system. In one aspect of this method the yield of the production phase is increased by about 10%. In one aspect of this method the yield of the production phase is increased by about 20%. In one aspect of this method the yield of the production phase is increased by about 30%. In one aspect of this method the yield of the production phase is increased by about 40%. In one aspect of this method the yield of the production phase is increased by about 50%. In one aspect of this method the yield of the production phase is increased by about 60%. In one aspect of this method the yield of the production phase is increased by about 70%. In one aspect of this method the yield of the production phase is increased by about 80%. In one aspect of this method the yield of the production phase is increased by about 90%. In one aspect of this method the yield of the production phase is increased by about 100%. In one aspect of this method the yield of the production phase is increased by about 200%. In one aspect of this method the yield of the production phase is increased by about 500%.

In another embodiment, the invention provides a method to produce a protein of interest at a temperature that is elevated compared to normal growth conditions for the production of that protein, comprising the addition of a supplement of the invention to cells expressing the protein of interest.

In another embodiment, the invention provides a method to decrease the amount of aggregates formed in a cell culture expression system by aggregate prone proteins of interest comprising the addition of a supplement of the invention to the cell culture expression system, whereby the aggregation state of the protein is reduced.

In another embodiment, the invention provides a method to increase the activity of a protein of interest protein expressed by a cell by preventing the denaturation and aggregation of the recombinant protein comprising the addition of a supplement of the invention to the cell, whereby the specific activity of the protein of interest is increased.

In another embodiment, the invention provides a method to improve the expression of proteins in a cell culture expression system that are aggregation prone, cause precipitation to occur, or are toxic themselves to the cells comprising the addition of a supplement of the invention to the cell culture expression system, whereby the expression of the protein of interest is increased.

In another embodiment, the invention provides a method to improve the glycosylation pattern of glycosylated proteins comprising the addition of a supplement of the invention to the cell culture expression system, whereby the degree of glycosylation is increased, and/or the pattern of glycosylation obtained is more human like.

The amount of supplement to add in any of these methods will depend on various factors, for instance, the type of host cell, the cell density, protein of interest and culture conditions, etc. Determining the desired concentration of supplement to be added to the culture media is within the skill in the art and can be ascertained empirically by routine optimization and without undue experimentation.

The skilled artisan will readily appreciate that different cell types will have different magnitudes of responses to the supplement of the invention, and this will be determined, to some degree, by the amount or type of the HSPs in the supplement. Additionally different densities of cells will require appropriate adjustment in the total amount of supplement as well as the concentration of HSPs added to the culture to account for the increased cell number. Additionally cells grown in suspension culture or via adherent culture will have different membrane surface areas available for HSP entry and will typically exhibit different rates and degrees of response. Therefore, one should choose a concentration which provides for a sufficient inhibition of apoptosis, or increase in viability, or net cell growth. Typically the supplements of the invention will be added to a final concentration of about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 40%, or about 50%. Wt/wt, or wt/volume.

There will typically be an upper range of concentration of the supplement beyond which further increases in cell survival do not occur. As described in the Examples below, Applicants have found that the supplements of the invention can inhibit apoptosis when added to cell cultures at a concentration of about 200 mg/L to about 2 g/L, or more preferably about 200 mg/L to about 1000 mg/L, or more preferable about 250 to about 500 mg/L.

### II. Supplements

In one aspect the supplements of the invention comprise one or more plant derived recombinant cell culture components. In one embodiment of the invention, the one or more recombinant cell culture components are independently selected from albumin and lactoferrin, or a mixture thereof.

In another aspect of the invention, the supplements contain one or more additional factors selected from the group consisting of transferrin, glutathione S-transferase, superoxide dismutase or a growth factor.

In another aspect of the invention, the growth factors are independently selected from insulin, Epidermal Growth Factor (EGF), Fibroblast Growth Factors 1-23 (FGF), Insulin-like Growth Factor-1 (IGF), keratinocyte growth factors 1 & 2(KGF), and Leukemia Inhibitory Factor (LIF).

In one aspect of the supplements of the invention, at least one of the recombinant cell culture components is albumin. In another aspect, the albumin comprises less than about 2% aggregated albumin. In another aspect the albumin comprises less than about 1% aggregated albumin.

In one aspect of the supplements of the invention, the recombinant cell culture components comprise a mixture of albumin and lactoferrin. In another aspect, the albumin comprises less than about 2% aggregated albumin. In another aspect the albumin comprises less than about 1% aggregated albumin.

In another aspect the supplements of the invention comprise recombinant albumin and a rice heat shock protein. In another aspect the supplements of the invention comprise recombinant albumin and a rice hsp70 homolog. In one aspect the rice hsp70 homolog is selected from HSP70, Bip and rice stromal protein.

In one embodiment, the supplements of the invention comprise preparations of the co-purified recombinant albumin and rice hsps that are also essentially free of detergents and endotoxins which would otherwise mask or inhibit the positive impact of the hsp. In one aspect the supplements of the invention have less than about 1 EU of endotoxin, and said albumin is at least about 95% pure.

In any of these methods the supplements of the invention may be prepared by co-purifying, or mixing in aqueous solution the cell culture components with a heat shock protein.

The term “albumin” refers to all naturally-occurring and synthetic forms of albumin. Preferably, the term “albumin” refers to recombinant albumin. In one aspect the albumin is from a vertebrate. In one aspect the albumin is from a mammal. In a further embodiment the albumin is human. In another aspect, the recombinant albumin is produced from a plant cell. In one particularly preferred embodiment the recombinant albumin is produced from transgenic rice (Oryza sativa). Representative species and Gene bank accession numbers for various species of albumin are listed below in Table D 1

TABLE D1
Exemplary Albumin genes

| Species | Gene Bank Accession number |
|---|---|
| Human | NP_000468.1 |
| Pan troglodytes | XP_517233.2 |
| Canis lupus familiaris | XP_855557.1 |
| Bos taurus | NP_851335.1 |
| Mus musculus | NP_033784.1 |
| Rattus norvegicus | NP_599153.1 |
| Gallus gallus | NP_990592.1 |

It will be understood that for the recombinant production of albumin in different species it will typically be necessary to codon optimize the nucleic acid sequence of the gene for the host organism in question. Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

The albumin may be in its native form, i.e., as different allelic variants as they appear in nature, which may differ in their amino acid sequence, for example, by truncation (e.g., from the N- or C-terminus or both) or other amino acid deletions, additions, insertions, substitutions, or post-translational modifications. Naturally-occurring chemical modifications including post-translational modifications and degradation products of the albumin, are also specifically included in any of the methods of the invention including for example, pyroglutamyl, iso-aspartyl, proteolytic, phosphorylated, glycosylated, reduced, oxidatized, isomerized, and deaminated variants of the albumin.

Fragments of native or synthetic albumin sequences may also have the desirable functional properties of the peptide from which they derived and may be used in any of the methods of the invention. The term “fragment” as used herein thus includes fragments of albumin provided that the fragment retains the biological or therapeutically beneficial activity of the whole molecule.

For example, albumin contains at least 2 high affinity multi-metal binding sites for a number of physiologically important metals ions including copper, zinc, cadmium and nickel. (Carter et al., Advances in Protein Chemistry 45 153-203 (1994); Bai et al., J. Inorg Biochem 70 (1) 33-39 (1998), Blindauer et al., J. Biol. Chem. 284 (34) 23116-24 (2009); U.S. Pat. No. 6,787,636). Since trace amounts of these metals are typically present in the recombinant production of albumin, a significant amount of these metal ions can be become chelated to the protein. The binding of these ions, and in particular the binding of cadmium and nickel to recombinant albumin is associated with cellular toxicity of the protein when added to cells as a tissue culture component.

Accordingly, in one aspect, the term albumin can comprise a fragment of albumin that includes the deletion of one or amino acids involved in the multi-metal binding sites of albumin. In one aspect the albumin fragment is created by the deletion of one or more amino acids at the N-terminus of the mature protein. In another aspect the albumin can comprise one or more deletions or mutations of any of the amino acids involved in the N-terminal metal binding site of albumin. In one aspect, the amino acids to be deleted or mutated are independently selected from the sequence 5′ DAHKSEVAH 3′ (SEQ. ID. NO. 1).

The term “derivative” as used herein thus refers to albumin sequences or fragments thereof, which have modifications as compared to the native sequence. Such modifications may be one or more amino acid deletions, additions, insertions and/or substitutions. These may be contiguous or non-contiguous. Representative variants may include those having 1 to 20, or more preferably 1 to 15, 1 to 10, or 1 to 5 amino acid substitutions, insertions, and/or deletions as compared to any of genes listed in Tables D1. The substituted amino acid may be any amino acid, particularly one of the well-known 20 conventional amino acids (Ala (A); Cys (C); Asp (D); Glu (E); Phe (F); Gly (G); His (H); Ile (I); Lys (K); Leu (L); Met (M); Asn (N); Pro (P); Gin (O); Arg (R); Ser (S); Thr (T); Val (V); Trp (W); and Tyr (Y)). Any such variant or derivative of albumin may be used in any of the methods of the invention.

Accordingly, the albumin of the invention can comprise amino acid deletions, insertions or mutations in any of the functional binding domains of albumin. In one aspect the albumin may comprise a mutation in a binding domain of albumin. In one aspect the mutated binding domain is a domain involved in the binding of aspirin, warfarin, diazepam, digitoxin, dlofibrate, ibuprofen or AZT, as outlined is U.S. Pat. No. 5,780,593, or a multimetal binding site as outlined in Blindauer et al., J. Biol. Chem. 284 (34) 23116-24 (2009).

Thus, the albumin which may be used in any of the methods of the invention may have amino acid sequences which are substantially homologous, or substantially similar to the native albumin amino acid sequences, for example, to any of the native albumin gene sequences listed in Table D1. Alternatively, the albumin may have an amino acid sequence having at least 30% preferably at least 40, 50, 60, 70, 75, 80, 85, 90, 95, 98, or 99% identity with albumin listed in Table D1. In a preferred embodiment, the albumin for use in any of the methods of the present invention is at least 80% identical to the mature secreted human serum albumin (SEQ. ID No. 2) as shown underlined in the below (Swiss-Prot P02768):

(SEQ. ID. NO. 2)
MKWVTFISLL FLFSSAYSRG VFRRDAHKSE VAHRFKDLGE ENFKALVLIA FAQYLQQCPF
EDHVKLVNEV TEFAKTCVAD ESAENCDKSL HTLFGDKLCT VATLRETYGE MADCCAKQEP
ERNECFLQHK DDNPNLPRLV RPEVDVMCTA FHDNEETFLK KYLYEIARRH PYFYAPELLF
FAKRYKAAFT ECCQAADKAA CLLPKLDELR DEGKASSAKQ RLKCASLQKF GERAFKAWAV
ARLSQRFPKA EFAEVSKLVT DLTKVHTECC HGDLLECADD RADLAKYICE NQDSISSKLK
ECCEKPLLEK SHCIAEVEND EMPADLPSLA ADFVESKDVC KNYAEAKDVF LGMFLYEYAR
RHPDYSVVLL LRLAKTYETT LEKCCAAADP HECYAKVFDE FKPLVEEPQN LIKQNCELFE
QLGEYKFQNA LLVRYTKKVP QVSTPTLVEV SRNLGKVGSK CCKHPEAKRM PCAEDYLSVV
LNQLCVLHEK TPVSDRVTKC CTESLVNRRP CFSALEVDET YVPKEFNAET FTFHADICTL
SEKERQIKKQ TALVELVKHK PKATKEQLKA VMDDFAAFVE KCCKADDKET CFAEEGKKLV
AASQAALGL

Fusion proteins of albumin to other proteins are also included, and these fusion proteins may enhance, activity, targeting, stability or potency.

Chemical modifications of the native albumin structure which retain or stabilize albumin activity or biological half-life may also be used with any of the methods described herein. Such chemical modification strategies include without limitation pegylation, glycosylation, and acylation (see Clark et al.: J. Biol. Chem. 271(36): 21969-21977, 1996; Roberts et al.: Adv. Drug. Deliv. Rev. 54(4): 459-476, (2002); Felix et al.: Int. J. Pept. Protein. Res. 46(3-4): 253-264, (1995); Garber Diabetes Obes. Metab. 7 (6) 666-74 (2005)) C- and N-terminal protecting groups and peptomimetic units may also be included.

Isomers of the native L-amino acids, e.g., D-amino acids may be incorporated in any of the above forms of albumin, and used in any of the methods of the invention. All such variants, derivatives, fusion proteins, or fragments of albumin are included, may be used in any of the methods claims or disclosed herein, and are subsumed under the term “albumin”.

The term “transferrin” refers to all naturally-occurring and synthetic forms of transferrin. In one aspect, the term “transferrin” refers to recombinant transferrin. In one aspect the transferrin is from a vertebrate. In one aspect the transferrin is from a mammal. In a further embodiment the transferrin is human. In another aspect the recombinant transferrin is produced from a plant cell. In one particularly preferred embodiment the recombinant transferrin is produced from transgenic rice (Oryza sativa). Representative species and Gene bank accession numbers for various species of transferrin are listed below in Table D2.

TABLE D2
Exemplary Transferrin genes

| Species | Gene Bank Accession number |
|---|---|
| Homo sapiens | NP_001054.1 |
| Canis lupus familiaris | XP_864550.1 |
| Bos taurus | NP_803450.2 |
| Mus musculus | NP_598738.1 |
| Rattus norvegicus | NP_001013128.1 |
| Gallus gallus | NP_990635.1 |
| Danio rerio | NP_001015057.1 |

The transferrin may be in its native form, i.e., as different apo forms, or allelic variants as they appear in nature, which may differ in their amino acid sequence, for example, by truncation (e.g., from the N- or C-terminus or both) or other amino acid deletions, additions, insertions, substitutions, or post-translational modifications. Naturally-occurring chemical modifications including post-translational modifications and degradation products of the transferrin, are also specifically included in any of the methods of the invention including for example, pyroglutamyl, iso-aspartyl, proteolytic, phosphorylated, glycosylated, reduced, oxidatized, isomerized, and deaminated variants of the transferrin.

The transferrin which may be used in any of the methods of the invention may have amino acid sequences which are substantially homologous, or substantially similar to the native transferrin amino acid sequences, for example, to any of the native transferrin gene sequences listed in Table D2. Alternatively, the transferrin may have an amino acid sequence having at least 30% preferably at least 40, 50, 60, 70, 75, 80, 85, 90, 95, 98, or 99% identity with transferrin listed in Table D2. In a preferred embodiment, the transferrin for use in any of the methods of the present invention is at least 80% identical to the mature human transferrin.

The term “Glutathione S-transferase” refers to all naturally-occurring and synthetic forms of Glutathione S-transferase. In one aspect, the term “Glutathione S-transferase” refers to recombinant Glutathione S-transferase. In one aspect the Glutathione S-transferase is from a vertebrate. In one aspect the Glutathione S-transferase is from a mammal. In a further embodiment the Glutathione S-transferase is human. In another aspect the recombinant Glutathione S-transferase is produced from a plant cell. In one particularly preferred embodiment the recombinant Glutathione S-transferase is produced from transgenic rice (Oryza sativa). Representative species and Gene bank accession numbers for various species of Glutathione S-transferase are listed below in Table D3.

TABLE D3
Exemplary Glutathione S-transferase genes

| Species | Gene Bank Accession number |
|---|---|
| Homo sapiens | NP_004519.1 |
| Pan troglodytes | XP_001174621.1 |
| Canis lupus familiaris | XP_536147.1 |
| Canis lupus familiaris | XP_851330.1 |
| Bos taurus | NP_001030218.1 |
| Mus musculus | NP_079845.1 |
| Rattus norvegicus | XP_213943.2 |
| Gallus gallus | XP_001232860.1 |
| Danio rerio | NP_ 998592.1 |
| Arabidopsis thaliana | NP_176758.1 |
| Oryza sativa | NP_001051042.1 |

The Glutathione S-transferase may be in its native form, i.e., as different apo forms, or allelic variants as they appear in nature, which may differ in their amino acid sequence, for example, by truncation (e.g., from the N- or C-terminus or both) or other amino acid deletions, additions, insertions, substitutions, or post-translational modifications. Naturally-occurring chemical modifications including post-translational modifications and degradation products of the Glutathione S-transferase, are also specifically included in any of the methods of the invention including for example, pyroglutamyl, iso-aspartyl, proteolytic, phosphorylated, glycosylated, reduced, oxidatized, isomerized, and deaminated variants of the Glutathione S-transferase. The Glutathione S-transferase which may be used in any of the methods of the invention may have amino acid sequences which are substantially homologous, or substantially similar to the native Glutathione S-transferase amino acid sequences, for example, to any of the native Glutathione S-transferase gene sequences listed in Table D2. Alternatively, the Glutathione S-transferase may have an amino acid sequence having at least 30% preferably at least 40, 50, 60, 70, 75, 80, 85, 90, 95, 98, or 99% identity with Glutathione S-transferase listed in Table D2. In a preferred embodiment, the Glutathione S-transferase for use in any of the methods of the present invention is at least 80% identical to the mature human Glutathione S-transferase.

The term “Superoxide Dismutase” refers to all naturally-occurring and synthetic forms of Superoxide Dismutase. In one aspect, the term “Superoxide Dismutase” refers to recombinant Superoxide Dismutase. In one aspect the Superoxide Dismutase is from a vertebrate. In one aspect the Superoxide Dismutase is from a mammal. In a further embodiment the Superoxide Dismutase is human. In another aspect the recombinant Superoxide Dismutase is produced from a plant cell. In one particularly preferred embodiment the recombinant Superoxide Dismutase is produced from transgenic rice (Oryza sativa). Representative species and Gene bank accession numbers for various species of Superoxide Dismutase are listed below in Table D4.

TABLE D4
Exemplary Superoxide Dismutase genes

| Species | Gene Bank Accession number |
|---|---|
| Homo sapiens | NP_000445.1 |
| Pan troglodytes | NP_001009025.1 |
| Canis lupus familiaris | NP_001003035.1 |
| Bos taurus | XP_584414.4 |
| Bos taurus | NP_777040.1 |
| Mus musculus | NP_035564.1 |
| Mus musculus | XP_994787.1 |
| Rattus norvegicus | NP_058746.1 |
| Gallus gallus | NP_990395.1 |
| Danio rerio | NP_571369.1 |
| Drosophila melanogaster | NP_476735.1 |
| Anopheles gambiae | XP_311594.2 |
| Caenorhabditis elegans | NP_494779.1 |
| Caenorhabditis elegans | NP_001021956.1 |
| Schizosaccharomyces pombe | NP_593163.1 |
| Saccharomyces cerevisiae | NP_012638.1 |
| Kluyveromyces lactis | XP_454197.1 |
| Eremothecium gossypii | NP_986346.1 |
| Magnaporthe grisea | XP_366549.2 |
| Neurospora crassa | XP_329323.1 |
| Arabidopsis thaliana | NP_001077494.1 |
| Oryza sativa | NP_001050118.1 |
| Oryza sativa | NP_001060564.1 |

The Superoxide dismutase may be in its native form, i.e., as different apo forms, or allelic variants as they appear in nature, which may differ in their amino acid sequence, for example, by truncation (e.g., from the N- or C-terminus or both) or other amino acid deletions, additions, insertions, substitutions, or post-translational modifications. Naturally-occurring chemical modifications including post-translational modifications and degradation products of the Superoxide dismutase, are also specifically included in any of the methods of the invention including for example, pyroglutamyl, iso-aspartyl, proteolytic, phosphorylated, glycosylated, reduced, oxidatized, isomerized, and deaminated variants of the Superoxide dismutase. The Superoxide dismutase which may be used in any of the methods of the invention may have amino acid sequences which are substantially homologous, or substantially similar to the native Superoxide dismutase amino acid sequences, for example, to any of the native Superoxide dismutase gene sequences listed in Table D4. Alternatively, the Superoxide dismutase may have an amino acid sequence having at least 30% preferably at least 40, 50, 60, 70, 75, 80, 85, 90, 95, 98, or 99% identity with Superoxide dismutase listed in Table D4. In a preferred embodiment, the Superoxide dismutase for use in any of the methods of the present invention is at least 80% identical to the mature human Superoxide dismutase.

The term “Lactoferrin” refers to all naturally-occurring and synthetic forms of Lactoferrin. In one aspect, the term “Lactoferrin” refers to recombinant Lactoferrin. In one aspect the Lactoferrin is from a vertebrate. In one aspect the Lactoferrin is from a mammal. In a further embodiment the Lactoferrin is human. In another aspect the recombinant Lactoferrin is produced from a plant cell. In one particularly preferred embodiment the recombinant Lactoferrin is produced from transgenic rice (Oryza sativa). Representative species and Gene bank accession numbers for various species of Lactoferrin are listed below in Table D5.

TABLE D5
Exemplary Lactoferrin genes

| Species | Gene Bank Accession number |
|---|---|
| Homo sapiens | AAA59511.1 |
| Sus scrofa | AAA31059.1 |
| Camelus dromedarius | CAB53387.1 |
| Bos taurus | AAA30610.1 |
| Equus caballus | CAA09407.1 |

The Lactoferrin may be in its native form, i.e., as different apo forms, or allelic variants as they appear in nature, which may differ in their amino acid sequence, for example, by truncation (e.g., from the N- or C-terminus or both) or other amino acid deletions, additions, insertions, substitutions, or post-translational modifications. Naturally-occurring chemical modifications including post-translational modifications and degradation products of Lactoferrin, are also specifically included in any of the methods of the invention including for example, pyroglutamyl, iso-aspartyl, proteolytic, phosphorylated, glycosylated, reduced, oxidatized, isomerized, and deaminated variants of Lactoferrin. Lactoferrin which may be used in any of the methods of the invention may have amino acid sequences which are substantially homologous, or substantially similar to the native Lactoferrin amino acid sequences, for example, to any of the native Lactoferrin gene sequences listed in Table D5. Alternatively, the Lactoferrin may have an amino acid sequence having at least 30% preferably at least 40, 50, 60, 70, 75, 80, 85, 90, 95, 98, or 99% identity with Lactoferrin listed in Table D5. In a preferred embodiment, the Lactoferrin for use in any of the methods of the present invention is at least 80% identical to the mature human Lactoferrin.

In one aspect, the supplements of the invention may be prepared by mixing the isolated cell culture components with a purified, or semi purified preparation of one or more heat shock proteins in aqueous solution. Such heat shock proteins will be typically be mixed in a molar ratio of the cell culture component to hsp of about 1:1, about 1:10, about 1:20, about 1:50, about 1:100, about 1:200, about 1:500, about 1:1000, or about 1:10,000. In one aspect a mixture of the cell culture component and one or more heat shock proteins may be incubated together in an aqueous buffer at about 4° C. to 25° C. for a time ranging from a few minutes to overnight. In another aspect a mixture of cell culture component and one or more heat shock proteins may be incubated together in an aqueous buffer at about 20° C. to about 37° C. for a time ranging from a few minutes to overnight. In one aspect the cell culture component and hsp may be mixed in the presence of ATP to enable the hsp to undergo ATP-dependent conformation binding to the cell culture component. In one aspect of any of these methods, the aqueous buffer has a pH of about 6.5 to about 7.5. In another aspect of any of these methods, the aqueous buffer solution comprises a buffer selected from phosphate, TRIS, HEPES, and acetate. In one aspect of any of these methods the complex of the cell culture component and the heat shock protein is isolated.

In one aspect of any of the claimed methods the cell culture component is albumin. In one aspect of any of the claimed methods the cell culture component is lactoferrin. In one aspect of any of the claimed methods the cell culture component is transferrin. In one aspect of any of the claimed methods the cell culture component is a human growth factor.

Liquids of known concentration can also be combined containing one component part A (albumin or another cell culture component), to a liquid containing part B (such as a heat shock protein) to obtain a ratio that contains approximately 0.01% to 0.5% wt/wt hsp with respect to cell culture component. Powdered, lyophilized, or otherwise dried powder (Hsp) can be added directly to an aqueous solution containing the cell culture component in order to obtain a ratio based on dry weight of Hsp at 0.01% to 0.5% Hsp with respect to cell culture component. Powdered, lyophilized, or otherwise dried Hsp can also be blended with the cell culture component powder on a mass to mass basis to obtain a ratio that is completely based on gravimetrics. The resulting powder can be dissolved at concentrations ranging from very low (picomolar) to very high concentrations (millimolar) in suitable buffers that are common to the art to reconstitute the cell culture component/hsp complex.

In one aspect, supplements of the present invention will accordingly comprise albumin and one or more heat shock proteins. Such supplements will commonly be prepared as sterile liquid or powder form. The total amount of hsp in the composition may vary from 1% to 0.001% of weight of the cell culture component. In other aspects the amount of hsp in the composition may vary from about 0.01% to about 0.02%, or about 0.01% to about 0.09%, or about 0.02% to about 0.04%, or about 0.02% to about 0.06,% or about 0.02% to about 0.08%.

In another aspect the amount of hsp in the composition is greater than about 0.02%, or more preferably greater than about 0.03%, or more preferably greater than about 0.04% wt/wt, or more preferably greater than about 0.05% wt/wt hsp with respect to the cell culture component.

In one aspect of any of the claimed supplements, the supplement is essentially free of endotoxin and detergents. In another aspect the supplement has less than about 1 EU/mg of endotoxin. In yet another aspect, the supplement contains less than about 10 ppm detergent. In another aspect of any of the claimed supplements, the cell culture component has a purity of greater than 95%.

In another of any of the claimed supplements, the supplement comprises recombinant albumin which is bound to a rice heat shock protein, wherein the complex has less than about 1 EU of endotoxin and is at least 95% pure. In one aspect the recombinant albumin is produced in rice.

In another aspect of any of these methods the supplement contains albumin as the cell culture component, and the albumin is essentially free of aggregated albumin. In another aspect of any of these supplements the albumin has less than about 2% aggregated albumin.

### III. Exemplary Heat Shock Proteins

The terms “heat shock protein”, “HSP” or “hsp”, as used herein includes all naturally-occurring and synthetic forms of the heat shock protein super family that retain anti-apoptotic activity. Such heat shock proteins include the small heat shock proteins/HSPB family, Hsp40/DnaJ family, HSP70/HSPA family, HSP90/HSPC family, HSP110/HSPH family and chapererone family, as well as peptide fragments and protein complexes of two or more heat shock proteins or nucleotide exchange factors (for example, complexes of HSP70 & HSP40) derived therefrom.

Heat shock genes from a large number of different species have been sequenced, and are known in the art to be at least partially functionally interchangeable. It would thus be a routine matter to select a variant being a heat shock protein from a family or species or genus other than rice heat shock protein. Several such variants of heat shock proteins (i.e., representative heat shock proteins) are shown in Tables D6-D8.

The heat shock proteins were originally identified as stress-responsive proteins required to adapt to thermal and other stresses. It became clear shortly thereafter that all HSP families also encode constitutively expressed members like Hsc70 (HSPA8) in the HSP70 family. The heat shock genes (and the protein family members that they encode) that have been most extensively studied are those that are heat inducible, such as HSP70i. (HSPA1A/B), HSP40 (DNAJB1), and HSP27 (HSPB1). Heat shock proteins, as a class, are among the most highly expressed cellular proteins across all species. As their name implies, heat shock proteins protect cells when stressed by elevated temperatures. They account for 1-2% of total protein in unstressed cells. However when cells are heated, the fraction of heat shock proteins increases to about 4-6% of cellular proteins.

The number of genes coding for the diverse HSP family members varies widely in different organisms. For example, in the HSPA (HSP70) family, the number of members varies from three in Escherichia coli to 13 in humans. Gene duplication during evolution likely satisfied the need for additional members in different intracellular compartments as well as for tissue specific or developmental expression. Moreover, gene duplication provides functional diversity for client specificity and/or processing.

All such homologues, orthologs, and naturally occurring isoforms of heat shock proteins from eukaryotes, prokaryotes, vertebrates, invertebrates, and plants as well as other species are included in any of the methods of the invention, as long as they retain detectable anti-apoptotic activity.

Since the annotation of the human genome, the names used for the Hsp family members in the literature have become chaotic and up to ten different names can be found for the same gene product. The nomenclature used in the tables below is based on the systematic gene symbols that have been assigned by the HUGO Gene Nomenclature Committee (HGNC) and are used as the primary identifiers in databases such as Entrez Gene and Ensemble. (Kaminga et al., Cell Stress 7 Chaperones 1.4 105-111 (2009)).

The human genome encodes 13 members of the HSPA family (Table D6), excluding the many pseudogenes. The most studied genes are HSPA1A and HSPA1B, the products of which only differ by two amino acids and which are believed to be fully interchangeable proteins. Together with HSPA6, these are the most heat-inducible family members. HSPA7 has long been considered to be a pseudogene, but recent analyses suggest that it might be a true gene that is highly homologous to HSPA6. HSPA8 is the cognate HSPA and was designated previously as Hsc70 (or HSP73). It is an essential “house-keeping” HSPA member and is involved in co-translational folding and protein translocation across intracellular membranes. HSPA1L and HSPA2 are two cytosolic family members with high expression in the testis. HSPA9 is the mitochondrial housekeeping HSPA member (HSPA9 is also known as mortalin/mtHSP70/GRP75/PBP74).

TABLE D6
HSP70 superfamily: HSPA (HSP70)

| | Gene | Protein | | Human | Mouse |
|---|---|---|---|---|---|
| HSP A | name | name | Old names | gene ID | ortholog ID |
| 1 | HSPA1A | HSPA1A | HSP70-1; HSP72; HSPA1 | 3303 | 193740 |
| 2 | HSPA1B | HSPA1B | HSP70-2 | 3304 | 15511 |
| 3 | HSPA1L | HSPA1L | hum70t; hum70t; Hsp-hom | 3305 | 15482 |
| 4 | HSPA2 | HSPA2 | Heat-shock 70kD protein-2 | 3306 | 15512 |
| 5 | HSPA5 | HSPA5 | BIP; GRP78; MIF2 | 3309 | 14828 |
| 6 | HSPA6 | HSPA6 | Heat shock 70kD protein 6 | 3310 | X |
| | | | (HSP70B′) | | |
| 7 | HSPA7a | HSPA7 | Heat shock 70kD protein 7 | 3311 | X |
| 8 | HSPA8 | HSPA8 | HSC70; HSC71; HSP71; HSP73 | 3312 | 15481 |
| 9 | HSPA9 | HSPA9 | GRP75; HSPA9B; MOT; | 3313 | 15526 |
| | | | MOT2; PBP74; mot-2 | | |
| 10 | HSPA12A | HSPA12A | FLJ13874; KIAA0417 | 259217 | 73442 |
| 11 | HSPA12B | HSPA12B | RP23-32L15.1; 2700081N06Rik | 116835 | 72630 |
| 12 | HSPA13b | HSPA13 | Stch | 6782 | 110920 |
| 13 | HSPA14 | HSPA14 | HSP70-4; HSP70L1; | 51182 | 50497 |
| | | | MGC131990 | | |

aAnnotated as pseudogene, but possibly a true gene
bUnder consultation with HGNC and the scientific community

Members of the Hsp70 family are strongly up-regulated by heat stress and toxic chemicals, particularly heavy metals such as arsenic, cadmium, copper, mercury, etc. Hsp70 was originally discovered by FM Ritossa in the 1960s when a lab worker accidentally boosted the incubation temperature of Drosophila (fruit flies). When examining the chromosomes, Ritossa found a “puffing pattern” that indicated the elevated gene transcription of an unknown protein. This was later described as the “Heat Shock Response”.

Hsp70 proteins play important roles in guiding the folding of new proteins, improving protein integrity, and also aid in the transmembrane transport of proteins, by stabilizing them in a partially-folded state. In addition to improving overall protein integrity, Hsp 70 also directly inhibits apoptosis, and participates in the recognition and disposal of damaged or defective proteins.

Consistent with Hsp70's central role in enhancing protein folding, the expression of Hsp 70 can also act to protect cells from thermal or oxidative stress during routine tissue culture processes such as cryopreservation and bio-processing. These stresses normally act to damage proteins, causing partial unfolding and possible aggregation. By temporarily binding to hydrophobic residues exposed by stress, Hsp70 prevents these partially-denatured proteins from aggregating, and allows them to refold. Low ATP which is characteristic of heat shock further enhances sustained binding of the HSP70 and further acts to enhance the ability of the HSPs to suppress aggregation. In a thermophile anaerobe (Thermotoga maritima) the Hsp70 demonstrates redox sensitive binding to model peptides, suggesting a second mode of binding regulation based on oxidative stress.

Hsp70 also inhibits apoptosis by blocking the recruitment of procaspase-9 leading to caspase 3 activation, and seems to be able to participate in disposal of damaged or defective proteins via interactions with CHIP (Carboxyl-terminus of Hsp70 Interacting Protein)—an E3 ubiquitin ligase.

Therefore, Hsp 70 proteins not only prevent damage to proteins, but also act to directly prevent programmed cell death under stressful conditions. The human genome also encodes four HSP110 (HSPH; Table D7) genes which encode a family of HSPs with high homology to HSPA members except for the existence of a longer linker domain between the N-terminal ATPase domain and the C-terminal peptide binding domain. In fact, two members, HSPA4 (HSPH2) and HSPA4L (HSPH3), were previously named as HSPA members in the Entrez Gene database. Besides the three cytosolic members, one compartment-specific HSPH member (HYOU1/Grp170) is present in the ER, (HSPH4). Recent evidence shows that HSPH members are nucleotide exchange factors for the HSPA family.

TABLE D7
HSP H superfamily: HSPH (HSP110)

| | | | | | Mouse |
|---|---|---|---|---|---|
| | Gene | Protein | | Human | ortholog |
| | name | name | Old names | gene ID | ID |
| 1 | HSPH1 | HSPH1 | HSP105 | 10808 | 15505 |
| 2 | HSPH2b | HSPH2 | HSPA4; APG-2; HSP110 | 3308 | 15525 |
| 3 | HSPH3b | HSPH3 | HSPA4L; APG-1 | 22824 | 18415 |
| 4 | HSPH4b | HSPH4 | HYOU1/Grp170; ORP150; | 10525 | 12282 |
| | | | HSP12A | | |

aAnnotated as pseudogene, but possibly a true gene
bUnder consultation with HGNC and the scientific community

In one embodiment of the any of the claims, the supplement of the invention comprises a Hsp selected from a small heat shock protein family member. In another aspect, the Hsp is selected from a HSP40/DnaJ family member. In another aspect, the Hsp is selected from a HSP70 family member. In another aspect, the Hsp is selected from a HSP90 family member. In another aspect, the Hsp is selected from a HSP110 family member. In another aspect, the Hsp is selected from a chapererone family member.

In one aspect of any the claims, the supplement of the invention comprises a Hsp superfamily member which is derived from a mammalian, insect, yeast or plant cell. In another aspect the Hsp superfamily member is derived from a plant cell. In yet another embodiment the HSP superfamily member is derived from rice (Oryza sativa).

In one aspect of any of these claims the supplement of the invention comprises a hsp superfamily member which is present in a protein complex with one or more other proteins. In a one aspect, the HSP superfamily member is complexed with another Hsp superfamily member of nucleotide exchange factor. In another aspect of any of these claims the Hsp superfamily member is bound to Albumin.

In one embodiment, the supplement of the invention comprises a HSP70 family member. In one aspect, the HSP70 family member is selected from HSPA1A (HSP72), HSPA8 (Hsc72) and HSPA9 (Grp78). In one aspect of any the claims, the HSP superfamily member is derived from a mammalian, insect, yeast or plant cell. In a preferred aspect the HSP superfamily member is derived from a plant cell. In one particularly preferred embodiment the HSP superfamily member is derived from rice (Oryza sativa).

In one aspect of any of the claims, the supplement of the invention comprises a HSP70 family member which is selected from a sequence from Table D8.

### HSPA1A

TABLE D8
HSPA1 HSP70 genes

| | |
|---|---|
| Human | 1 MAKAAAIGID LGTTYSCVGV FQHGKGERNV LIFDLGGGTF DVSILTIDDG IFEVKATAGD |
| CAM24989 | 61 THLGGEDFDN RLVNHFVEEF KRKHKKDISQ NKRAVRRLRT ACERAKRTLS SSTQASLEID |
| | 121 SLFEGIDFYT SITRARFEEL CSDLFRSTLE PVEKALRDAK LDKAQIHDLV LVGGSTRIPK |
| | 181 VQKLLQDFFN GRDLNKSINP DEAVAYGAAV QAAILMGDKS ENVQDLLLLD VAPLSLGLET |
| | 241 AGGVMTALIK RNSTIPTKQT QIFTTYSDNQ PGVLIQVYEG ERAMTKDNNL LGRFELSGIP |
| | 301 PAPRGVPQIE VTFDIDANGI LNVTATDKST GKANKITITN DKGRLSKEEI ERMVQEAEKY |
| | 361 KAEDEVQRER VSAKNALESY AFNMKSAVED EGLKGKISEA DKKKVLDKCQ EVISWLDANT |
| | 421 LAEKDEFEHK RKELEQVCNP IISGLYQGAG GPGPGGFGAQ GPKGGSGSGP TIEEVD |
| | (SEQ. ID. NO. 3) |
| Insect | 1 MPAIGIDLGT TYSCVGVYQH GKVEIIANDQ GNRTTPSYVA FTDSERLIGD PAKNQVAMNP |
| NP_524798 | 61 RNTVFDAKRL IGRKYDDPKI AEDMKHWPFK VVSDGGKPKI GVEYKGESKR FAPEEISSMV |
| | 121 LTKMKETAEA YLGESITDAV ITVPAYFNDS QRQATKDAGH IAGLNVLRII NEPTAAALAY |
| | 181 GLDKNLKGER NVLIFDLGGG TFDVSILTID EGSLFEVRST AGDTHLGGED FDNRLVTHLA |
| | 241 DEFKRKYKKD LRSNPRALRR LRTAAERAKR TLSSSTEATI EIDALFEGQD FYTKVSRARF |
| | 301 EELCADLFRN TLQPVEKALN DAKMDKGQIH DIVLVGGSTR IPKVQSLLQD FFHGKNLNLS |
| | 361 INPDEAVAYG AAVQAAILSG DQSGKIQDVL LVDVAPLSLG IETAGGVMTK LIERNCRIPC |
| | 421 KQTKTFSTYA DNQPGVSIQV YEGERAMTKD NNALGTFDLS GIPPAPRGVP QIEVTFDLDA |
| | 481 NGILNVSAKE MSTGKAKNIT IKNDKGRLSQ AEIDRMVNEA EKYADEDEKH RQRITSRNAL |
| | 541 ESYVFNVKQA VEQAPAGKLD EADKNSVLDK CNDTIRWLDS NTTAEKEEFD HKLEELTRHC |
| | 601 SPIMTKMHQQ GAGAGAGGPG ANCGQQAGGF GGYSGPTVEE VD |
| | (SEQ. ID. NO. 4) |
| Yeast | 1 MSRAVGIDLG TTYSCVAHFS NDRVEIIAND QGNRTTPSYV AFTDTERLIG DAAKNQAAIN |
| NP_009478 | 61 PHNTVFDAKR LIGRKFDDPE VTTDAKHFPF KVISRDGKPV VQVEYKGETK TFTPEEISSM |
| | 121 VLSKMKETAE NYLGTTVNDA VVTVPAYFND SQRQATKDAG TIAGMNVLRI INEPTAAAIA |
| | 181 YGLDKKGRAE HNVLIFDLGG GTFDVSLLSI DEGVFEVKAT AGDTHLGGED FDNRLVNHLA |
| | 241 TEFKRKTKKD ISNNQRSLRR LRTAAERAKR ALSSSSQTSI EIDSLFEGMD FYTSLTRARF |
| | 301 EELCADLFRS TLEPVEKVLK DSKLDKSQID EIVLVGGSTR IPKIQKLVSD FFNGKEPNRS |
| | 361 INPDEAVAYG AAVQAAILTG DQSTKTQDLL LLDVAPLSLG IETAGGIMTK LIPRNSTIPT |
| | 421 KKSETFSTYA DNQPGVLIQV FEGERTRTKD NNLLGKFELS GIPPAPRGVP QIDVTFDIDA |
| | 481 NGILNVSALE KGTGKSNKIT ITNDKGRLSK DDIDRMVSEA EKYRADDERE AERVQAKNQL |
| | 541 ESYAFTLKNT INEASFKEKV GEDDAKRLET ASQETIDWLD ASQAASTDEY KDRQKELEGI |
| | 601 ANPIMTKFYG AGAGAGPGAG ESGGFPGSMP NSGATGGGED TGPTVEEVD |
| | (SEQ. ID. NO. 5) |
| Rice | 1 MAGKGEGPAI GIDLGTTYSC VGVWQHDRVE IIANDQGNRT TPSYVGFTDS ERLIGDAAKN |
| NP_001068540 | 61 QVAMNPINTV FDAKRLIGRR FSDASVQSDI KLWPFKVIAG PGDKPMIVVQ YKGEEKQFAA |
| | 121 EEISSMVLIK MREIAEAYLG TTIKNAVVTV PAYFNDSQRQ ATKDAGVIAG LNVMRIINEP |
| | 181 TAAAIAYGLD KKATSVGEKN VLIFDLGGGT FDVSLLTIEE GIFEVKATAG DTHLGGEDFD |
| | 241 NRMVNHFVQE FKRKNKKDIT GNPRALRRLR TACERAKRTL SSTAQTTIEI DSLYEGIDFY |
| | 301 STITRARFEE LNMDLFRKCM EPVEKCLRDA KMDKSSVHDV VLVGGSTRIP RVQQLLQDFF |
| | 361 NGKELCKNIN PDEAVAYGAA VQAAILSGEG NEKVQDLLLL DVTPLSLGLE TAGGVMTVLI |
| | 421 PRNTTIPTKK EQVFSTYSDN QPGVLIQVYE GERTRTRDNN LLGKFELSGI PPAPRGVPQI |
| | 481 TVCFDIDANG ILNVSAEDKT TGQKNKITIT NDKGRLSKEE IEKMVQEAEK YKSEDEEHKK |
| | 541 KVESKNALEN YAYNMRNTIK DEKIASKLPA ADKKKIEDAI DQAIQWLDGN QLAEADEFDD |
| | 601 KMKELEGICN PIIAKMYQGA GADMAGGMDE DDAPPAGGSG AGPKIEEVD |
| | (SEQ. ID. NO. 6) |
| Rice | 1 MAGNKGEGPA IGIDLGTTYS CVGVWQHDRV EIIANDQGNR TTPSYVAFTD TERLIGDAAK |
| Os03g0277300 | 61 NQVAMNPTNT VFDAKRLIGR RFSDPSVQAD MKMWPFKVVP GPADKPMIVV TYKGEEKKFS |
| NP_001049719.1\| | 121 AEEISSMVLT KMKEIAEAFL STTIKNAVIT VPAYFNDSQR QATKDAGVIS GLNVMRIINE |
| | 181 PTAAAIAYGL DKKAASTGEK NVLIFDLGGG TFDVSILTIE EGIFEVKATA GDTHLGGEDF |
| | 241 DNRMVNHFVQ EFKRKHKKDI TGNPRALRRL RTACERAKRT LSSTAQTTIE IDSLYEGIDF |
| | 301 YATITRARFE ELNMDLFRRC MEPVEKCLRD AKMDKAQIHD VVLVGGSTRI PKVQQLLQDF |
| | 361 FNGKELCKSI NPDEAVAYGA AVQAAILSGE GNQRVQDLLL LDVTPLSLGL ETAGGVMTVL |
| | 421 IPRNTTIPTK KEQVFSTYSD NQPGVLIQVY EGERTRTKDN NLLGKFELTG IPPAPRGVPQ |
| | 481 INVTFDIDAN GILNVSAEDK TTGKKNKITI TNDKGRLSKE EIERMVQEAE KYKAEDEQVR |
| | 541 HKVEARNALE NYAYNMRNTV RDEKIASKLP ADDKKKIEDA IEDAIKWLDG NQLAEADEFE |
| | 601 DKMKELESLC NPIISKMYQG GAGGPAGMDE DAPNGSAGTG GGSGAGPKIE EVD |
| | (SEQ. ID. NO. 7) |
| Tobacco | 1 MAGKGEGPAI GIDLGTTYSC VGVWQHDRVE IIANDQGNRT TPSYVGFTDS ERLIGDAAKN |
| AAR17080 | 61 QVAMNPINTV FDAKRLIGRR FSDASVQSDI KLWPFKVISG PGDKPMIVVN YKGEEKQFAA |
| | 121 EEISSMVLIK MKEIAEAFLG STVKNAVVTV PAYFNDSQRQ ATKDAGVISG LNVMRIINEP |
| | 181 TAAAIAYGLD KKATSVGEKN VLIFDLGGGT FDVSLLTIEE GIFEVKATAG DTHLGGEDFD |
| | 241 NRMVNHFVQE FKRKHKKDIT GNPRALRRLR TACERAKRTL SSTAQTTIEI DSLYEGVDFY |
| | 301 STITRARFEE LNMDLFRKCM EPVEKCLRDA KMDKSTVHDV VLVGGSTRIP KVQQLLQDFF |
| | 361 NGKELCKSIN PDEAVAYGAA VQAAILSGEG NEKVQDLLLL DVTPLSLGLE TAGGVMTVLI |
| | 421 PRNTTIPTKK EQVFSTYSDN QPGVLIQVYE GERARTRDNN LLGKFELSGI PPAPRGVPQI |
| | 481 TVCFDIDANG ILNVSAEDKT TGQKNKITIT NDKGRLSKEE IEKMVQEAEK YKAEDEEHKK |
| | 541 KVEAKNALEN YAYNMRNTIK DEKIGSKLSS DDKKKIEDAI DQAISWLDSN QLAEADEFED |
| | 601 KMKELESICN PIIAKMYQGA GGEAGAPMDD DAPPAGGSSA GPKIEEVD |
| | (SEQ. ID. NO. 8) |

The heat shock proteins may be in their native form, i.e., as different variants as they appear in nature in different species which may be viewed as functionally equivalent variants, or they may be functionally equivalent natural derivatives thereof, which may differ in their amino acid sequence, for example, by truncation (e.g., from the N- or C-terminus or both) or other amino acid deletions, additions, insertions, substitutions, or post-translational modifications. Naturally-occurring chemical derivatives, including post-translational modifications and degradation products of the HSPs, are also specifically included in any of the methods of the invention including for example, pyroglutamyl, iso-aspartyl, proteolytic, phosphorylated, glycosylated, oxidatized, isomerized, and deaminated variants of the HSP.

It is known in the art to synthetically modify the sequences of proteins or peptides, while retaining their useful activity and this may be achieved using techniques which are standard in the art and widely described in the literature, e.g., random or site-directed mutagenesis, cleavage, and ligation of nucleic acids, or via the chemical synthesis or modification of amino acids or polypeptide chains.

The term “derivative” as used herein thus refers to HSP sequences or fragments thereof, which have modifications as compared to the native sequence. Such modifications may be one or more amino acid deletions, additions, insertions and/or substitutions. These may be contiguous or non-contiguous. Representative variants may include those having 1 to 100, or more preferably 1 to 50, 1 to 25, or 1 to 10 amino acid substitutions, insertions, and/or deletions as compared to any of genes listed in Tables D6 to D8. The substituted amino acid may be any amino acid, particularly one of the well-known 20 conventional amino acids (Ala (A); Cys (C); Asp (D); Glu (E); Phe (F); Gly (G); His (H); Ile (I); Lys (K); Leu (L); Met (M); Asn (N); Pro (P); Gin (Q); Arg (R); Ser (S); Thr (T); Val (V); Trp (W); and Tyr (Y)). Any such variant or derivative of a HSP may be used in any of the methods of the invention.

Thus, Hsps which may be used in any of the methods of the invention may have amino acid sequences which are substantially homologous, or substantially similar to the native HSP amino acid sequences, for example, to any of the native HSP gene sequences listed Tables D6 to D8. Alternatively, the HSP may have an amino acid sequence having at least 30% preferably at least 40, 50, 60, 70, 75, 80, 85, 90, 95, 98, or 99% identity with the amino acid sequence of any one of genes shown in Tables D6 to D8. In a one embodiment, the HSP for use in any of the methods of the present invention is at least 80% identical to a sequence selected from Table D6. In another embodiment, the HSP for use in any of the methods of the present invention is at least 80% identical to a sequence selected from Tables D6 to D8. In another aspect, the HSP for use in any of the methods of the invention is at least 80% identical to an Hspa8 gene selected from Table D8.

Fusion proteins of HSP to other proteins are also included, and these fusion proteins may enhance HSP biological activity, targeting, biological life, or stability.

Chemical modifications of the native HSP structure which retain or stabilize HSP activity or biological half-life may also be used with any of the methods described herein. Such chemical modification strategies include without limitation pegylation, glycosylation, and acylation (see Clark et al.: J. Biol. Chem. 271(36): 21969-21977, 1996; Roberts et al.: Adv. Drug. Deliv. Rev. 54(4): 459-476, (2002); Felix et al.: Int. J. Pept. Protein. Res. 46(3-4): 253-264, (1995); Garber Diabetes Obes. Metab. 7 (6) 666-74 (2005)) C- and N-terminal protecting groups and peptomimetic units may also be included.

Isomers of the native L-amino acids, e.g., D-amino acids may be incorporated in any of the above forms of HSP, and used in any of the methods of the invention. Additional variants may include amino and/or carboxyl terminal fusions as well as intrasequence insertions of single or multiple amino acids. Longer peptides may comprise multiple copies of one or more of the HSP peptide sequences. Insertional amino acid sequence variants are those in which one or more amino acid residues are introduced at a site in the protein.

Fragments of native or synthetic HSP sequences may also have the desirable functional properties of the peptide from which they derived and may be used in any of the methods of the invention. The term “fragment” as used herein thus includes fragments of a HSP provided that the fragment retains the biological or therapeutically beneficial activity of the whole molecule. Deletional variants are characterized by the removal of one or more amino acids from the sequence. Variants may also include, for example, different allelic variants as they appear in nature, e.g., in other species or due to geographical variation. All such variants, derivatives, fusion proteins, or fragments of HSP are included, may be used in any of the methods claims or disclosed herein, and are subsumed under the terms “heat shock protein” or “hsp”.

The variants, derivatives, and fragments are functionally equivalent in that they have detectable anti-apoptotic activity. More particularly, they exhibit at least 40%, preferably at least 60%, more preferably at least 80% of the activity of HSP70, particularly rice HSP70. Thus they are capable of functioning as anti-apoptotic agents when co-administered with albumin, i.e., can substitute for HSP70 itself.

Such activity means any activity exhibited by a native rice HSP, whether a physiological response exhibited in an in vivo or in vitro test system, or any biological activity or reaction mediated by a native HSP, for example, in an enzyme assay, cell growth assay or by testing the effect of the hsp on cell viability in the presence of stress.

Thus the activity of HSPs can be readily assessed using any previously disclosed methods to determine cell viability and apoptosis which are applicable to any cells grown in culture that can be conditioned to a serum free or low serum containing media or alternatively to media that contains components that are apoptotic or toxic in nature.

Additionally, growth rates may be determined using cells conditioned to grow in low serum or serum free conditions by plating defined numbers of the cells into multiwall plates. Cells may be seeded at different densities depending on the cells, and tissue culture plates employed. For example, hybridoma cells conditioned to serum free conditions can be seeded at an initial density of 0.5×10⁵ cells per mL of media. Typically the cells are initially washed three times, and specific ingredients required to support growth in culture such as albumin, candidate hsps, Glutathione S transferase, Superoxide Dismutase, or transferrin are added at the desired concentration in phosphate buffered saline up to about 1 part per 10 parts liquid media (for example, Dulbecos). At the end of the growth period at 37 C and 5% CO2, the cells are enumerated for viability. Dual label staining is the preferred method for determining viability in a mixture of viable and non-viable cells. The preferred method of determining the number of viable cells with respect to the total number of cells (percent viability) is to use a cell counting apparatus which is common to the art. Other methods that can be employed include dual label flow cytometry or alternatively manual counting of the cells utilizing a microscope, with stained with trypan blue and a cell counting device. Experimental sample viability and cell number are compared to the negative control, the media components minus the experimental factor(s), and the positive control (fetal bovine serum or other known cell culture supporting ingredients). In general, the statistical significance of the counts must be determined based on the signal to noise ratio of the replicate samples as well as the observed difference or lack of difference as compared to the positive and negative control. For those skilled in the art, with consideration that a stable cell platform must be established that allows serum free growth, a 20% change in viability versus the controls would be considered a significant difference with approximately 95% confidence provided a low signal to noise ratio for the replicate samples.

Performance of the potential factors may also be measured according to indicators of productivity including production of an endogenous or intentionally expressed protein or alternatively measured as a function of apoptotic indicators. Apoptosis assays are numerous and rely on upstream changes in the cell such as DNA fragmentation and nuclear degradation. Downstream assays rely on measurement of the activity of such apoptotic pathway components as Caspase 3. Cultured cells as conditioned in the previous method can also be assayed with a commercially available apoptosis assays to determine the effect of the added components to cell culture.

### IV. Production of Tissue Components

Albumin and other protein factors for use in the supplements of the present invention can be prepared in any suitable manner, for instance by isolation from naturally occurring sources, from genetically engineered host cells comprising expression systems (see below), or by chemical synthesis, using, for instance, automated peptide synthesizers, or any combination of such methods. The means for preparing such polypeptides are well understood in the art.

For recombinant production, host cells can be genetically engineered to incorporate nucleic acids encoding the culture component and/or a hsp of interest. Typically the nucleic acid will be codon optimized for high level expression in the expression system of choice, and incorporated into an expression vector to enable the expression of the protein of interest in the host cell. Vectors can exist as circular, double stranded DNA, and range in size form a few kilobases (kb) to hundreds of kb. Preferred cloning vectors have been modified from naturally occurring plasmids to facilitate the cloning and recombinant manipulation of polynucleotide sequences. Many such vectors are well known in the art and commercially available; see for example, by Sambrook (In. “Molecular Cloning: A Laboratory Manual,” second edition, edited by Sambrook, Fritsch, & Maniatis, Cold Spring Harbor Laboratory, (1989)), Maniatis, In: Cell Biology: A Comprehensive Treatise, Vol. 3, Gene Sequence Expression, Academic Press, NY, pp. 563-608 (1980).

In one aspect, expression vectors are used to increase the expression of the culture component in the host cell, while the expression of the host cells endogenous heat shock proteins is accomplished by activating the expression of the host cells genes. In another aspect expression vectors are used to increase the expression of the heat shock protein. In another aspect expression vectors are used to increase the expression of the heat shock protein and the cell culture component. In another aspect the nucleic acid sequence encoding a heat shock protein and the cell culture component are located in the same expression vector.

Expression vectors include plasmids, episomes, cosmids retroviruses or phages; the expression vector can be used to express a DNA sequence encoding the cell culture component or a hsp, and in one aspect comprises an assembly of expression control sequences. The choice of promoter and other regulatory elements can vary according to the intended host cell, and many such elements are available commercially, and can be readily assembled from isolated components such as the Gateway system from Invitrogen, (CA, USA). Expression systems for hsps or tissue culture components can be stable or transient expression systems.

In one aspect of any of these methods, hsp expression can be inducible, in another aspect, hsp expression can be constitutive. Inducible expression systems for hsps can be included in the expression vector for albumin, or can be included in a separate expression system or vector.

In one aspect of any of these methods, cell culture component expression can be inducible, in another aspect, hsp expression can be constitutive. Inducible expression systems for the tissue culture components can be included in the expression vector for the hsp, or can be included in a separate expression system or vector.

General and specific techniques for producing proteins from plant cells may be obtained from the following patents and applications, each of which is incorporated herein in its entirety by reference: U.S. Pat. Appi. Pub. No. 2003/0172403 A1 (“Plant Transcription Factors and Enhanced Gene Expression”); U.S. Pat. No. 6,991,824 (“Expression of Human Milk Proteins in Trans genic Plants”); U.S. Pat. Appi. Pub. No. 2003/0221223 (“Human Blood Proteins Expressed in Monocot Seeds”); U.S. Pat. Appl. Pub. No. 2004-0078851 (“Production of Human Growth Factors in Monocot Seeds”); U.S. Pat. Appl. Pub. No. 2004/0063617 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”). Other general and specific techniques for producing proteins from plant cells may be obtained, for example, from the following references, each of which is incorporated herein in its entirety by reference: U.S. Pat. No. 5,693,507, U.S. Pat. No. 5,932,479, U.S. Pat. Nos. 6,642,053, and 6,680,426 (each titled “Genetic Engineering of Plant Chloroplasts”); U.S. Pat. Appi. Pub. No. 2005/0066384 (“Site-Targeted Transformation Using Amplification Vectors”); U.S. Pat. Appi. Pub. No. 2005/0221323 (“Amplification Vectors Based on Trans-Splicing”); U.S. Pat. Appl. Pub. No. 2006/0026718 (“Method of Controlling Cellular Processes in Plants”); and U.S. Pat. Appi. Pub. No. 2006/0075524 (Method of Controlling A Cellular Process in a Multi-Cellular Organism”); Marillonnet et at., Systemic Agrobacterium tumefaciens-mediated transfection of viral replicons for efficient transient expression in plants, Nature Biotech. (2005) 23(6): 718-723.

Representative commercially available viral expression vectors include, but are not limited to, the adenovirus-based systems, such as the Per.C6 system available from Crucell, Inc., lentiviral-based systems such as pLP1 from Invitrogen, and retroviral vectors such as tobacco mosaic virus based vectors (Lindbo et al., BMC Biotechnol. (2007) 7 52-58).

An episomal expression vector is able to replicate in the host cell, and persists as an extrachromosomal episome within the host cell in the presence of appropriate selective pressure. (See for example, Conese et al., Gene Therapy 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP), specific examples include the vectors pREP4, pCEP4, pREP7 from Invitrogen. The host range of EBV based vectors can be increased to virtually any eukaryotic cell type through the co-expression of EBNA1 binding protein 2 (EPB2) (Kapoor et al., EMBO. J. 20: 222-230 (2001)), vectors pcDNA3.1 from Invitrogen, and pBK-CMV from Stratagene represent non-limiting examples of an episomal vector that uses T-antigen and the SV40 origin of replication in lieu of EBNA1 and oriP.

An integrating expression vector can randomly integrate into the host cell's DNA, or can include a recombination site to enable the specific recombination between the expression vector and the host cells chromosome. Such integrating expression vectors can utilize the endogenous expression control sequences of the host cell's chromosomes to effect expression of the desired protein. Examples of vectors that integrate in a site specific manner include, for example, components of the flp-in system from Invitrogen (e.g., pcDNA™ 5/FRT), or the cre-lox system, such as can be found in the pExchange-6 Core Vectors from Stratagene. Examples of vectors that integrate into host cell chromosomes in a random fashion include, for example, pcDNA3.1 (when introduced in the absence of T-antigen) from Invitrogen, pCI or pFN10A (ACT) FLEXI® from Promega.

Alternatively, the expression vector can be used to introduce and integrate a strong promoter or enhancer sequences into a locus in the cell so as to modulate the expression of an endogenous gene of interest such as a heat shock protein (Capecchi M R. Nat Rev Genet. (2005); 6 (6):507-12; Schindehutte et al., Stem Cells (2005); 23 (1):10-5). This approach can also be used to insert an inducible promoter, such as the Tet-On promoter (U.S. Pat. Nos. 5,464,758 and 5,814,618), in to the genomic DNA of the cell so as to provide inducible expression of an endogenous gene of interest, such as a heat shock protein. The activating construct can also include targeting sequence(s) to enable homologous or non-homologous recombination of the activating sequence into a desired locus specific for the gene of interest (see for example, Garcia-Otin & Guillou, Front Biosci. (2006) 11:1108-36). Alternatively, an inducible recombinase system, such as the Cre-ER system, can be used to activate a transgene in the presence of 4-hydroxytamoxifen (Indra et al. Nuc. Acid. Res. (1999) 27 (22): 4324-4327; Nuc. Acid. Res. (2000) 28(23): e99; and U.S. Pat. No. 7,112,715).

Alternatively in one embodiment, the host cell may endogenously express the hsp of interest or be induced to express the hsp of interest by the means described above such as, but not limited to, heat elevation. Polynucleotides may be introduced into host cells by methods described in many standard laboratory manuals, such as Davis et al., Basic Methods in Molecular Biology (1986) and Sambrook et al., (In. “Molecular Cloning: A Laboratory Manual,” second edition, edited by Sambrook, Fritsch, & Maniatis, Cold Spring Harbor Laboratory, (1989)), Maniatis, In: Cell Biology: A Comprehensive Treatise, Vol. 3, Gene Sequence Expression, Academic Press, NY, pp. 563-608 (1980). Exemplary methods of introducing polynucleotides into host cells include, for instance, calcium phosphate transfection, DEAE-dextran mediated transfection, transfection, microinjection, cationic lipid-mediated transfection, electroporation, transduction, scrape loading, ballistic introduction or infection.

Suitable cells for producing the tissue culture component and heat shock proteins include prokaryotic cells, yeasts, insect cells, plant expression systems and mammalian expression systems. Within these general guidelines, useful microbial hosts include, but are not limited to, bacteria from the genera Bacillus, Escherichia (such as E. coli), Pseudomonas, Streptomyces, Salmonella, Erwinia, Bacillus subtilis, Bacillus brevis, the various strains of Escherichia coli (e.g., HB101, (ATCC NO. 33694) DH5α, DH10 and MC1061 (ATCC NO. 53338)).

Many strains of yeast cells known to those skilled in the art are also available as host cells for the expression of albumin and hsps including those from the genera Hansenula, Kluyveromyces, Pichia, Rhino-sporidium, Saccharomyces, and Schizosaccharomyces, and other fungi. Preferred yeast cells include, for example, Saccharomyces cerivisae and Pichia pastoris.

Additionally, where desired, insect cell systems can be utilized in the methods of the present invention. Such systems are described, for example, by Kitts et al., Biotechniques, 14:810-817 (1993); Lucklow, Cum Opin. Biotechnol., 4:564-572 (1993); and Lucklow et al. (J. Virol., 67:4566-4579 (1993). Preferred insect cells include Sf-9 and HI5 (Invitrogen, Carlsbad, Calif.).

Many suitable plant expression systems can be used for the expression of albumin and hsps examples includes for example, any monocot or dicot plant. Suitable monocot plants include without limitation, rice, barley, wheat, rye, corn, millet, triticale, or sorghum, preferably rice. Other suitable plants include Arabidopsis, Alfalfa, tobacco, peanut and soybean.

A number of suitable mammalian host cells are also known in the art and many are available from the American Type Culture Collection (ATCC), 10801 University Boulevard, Manassas, Va. 20110-2209. Examples include, but are not limited to, mammalian cells, such as Chinese hamster ovary cells (CHO) (ATCC No. CCL61) CHO DHFR-cells (Urlaub et al., Proc. Natl. Acad. Sci. USA, 97:4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines.

Cell-free transcription and translation systems can also be employed to produce such proteins using the DNA constructs (or RNAs derived from the DNA constructs) of the present invention.

Accordingly, in another aspect, the invention comprises a method for producing a supplement with the ability to enhance survival and/or growth of cells or tissues in culture. The method comprises culturing a host cell of the invention under conditions sufficient for the expression of both cell culture component, and a heat shock protein and recovering the complex of albumin and the heat shock protein.

Production of recombinant proteins of the present invention may be prepared by processes well known in the art from genetically engineered host cells comprising expression systems. Accordingly, in a further aspect, the present invention relates to expression systems comprising a polynucleotide or polynucleotides encoding albumin and to host cells which are genetically engineered with such expression systems and to the production of such proteins by recombinant techniques. In one embodiment the host cell endogenously expresses a heat shock protein of interest.

In cases where purification of the expressed proteins of the supplement of the invention are necessary, proteins of the present invention can be recovered from either the cellular environment, before lysing the cells, or after cell lysis. The proteins can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. High performance liquid chromatography is also employed for purification.

Methods for the purification of heat shock proteins, including anion exchange chromatography and ATP agarose affinity chromatography are well known in the art. (Welch & Feramisco, J. Biol. Chem. 257 (24)14949-14959; (1982); Welch & Feramisco, Mol. Cell. Biol. 5 (6) 1229-1237 (1985). Well known techniques for refolding proteins may be employed to regenerate active conformation when the polypeptide is denatured during intracellular synthesis, isolation and/or purification.

A search of patents, published patent applications, and related publications will also provide those skilled in the art reading this disclosure with significant possible methods for preparing and purifying albumin. For example, U.S. Pat. Nos. 4,075,197; 4,086,222; 4,093,612; 4,097,473; 4,136,094; 4,228,154; 5,250,662; 5,656,729; 5,677,424; 5,710,253; 5,728,553; 5,994,507; 6,001,974; 6,638,740; 6,617,133 and 7,423,124 disclose various processes for purifying albumin. In one aspect, the albumin for use in the present invention is purified using any of these art recognized processes listed above, and then mixed in aqueous solution with a heat shock protein.

In one preferred aspect recombinant albumin is purified using procedures that enable the direct co-purification of both recombinant albumin and a heat shock protein, or hsp protein complex. In one aspect the recombinant albumin is produced in rice, and the heat shock protein is an endogenous rice heat shock protein.

Due to the similar electronegativity of albumin and hsp70, anion exchange chromatography is the preferred method to prepare albumin enriched in Hsps. For example, both albumin and Hsp70 bind to anion exchange columns with resins consisting of either quaternary amine or diethylaminoethyl mounted on a bead that is suitable for the ion exchange of polypeptides (large molecular exclusion limit and of suitable size) at high pH (7.5 and above). Examples of such resins are General Electric (GE) Q Sepharose and GE DEAE Sepharose. Due to their similar electronegativity, utilizing low pH conditions (below pH 6.5) allows for the co-purification of the two molecules on cation exchangers as well. Examples of such cation exchangers are GE Carboxymethyl Sepharose and Sulfonic acid Sepharose based resins. Because the albumin and Hsp70 have similar isoelectric points, mixed mode resins may also be employed for the co-purification of albumin and Hsp70. Since both Hsp70 and Albumin are well known to bind to fatty acids and other hydrophobic molecules, it is also possible to co-purify albumin and Hsp70 on a hydrophobic based resin such as octyl sepharose (GE). Due the similar size of Hsp70 proteins and Albumin (65-75 kDa), co-purification of the two proteins and enrichment of Hsp70 by tangential flow ultrafiltration utilizing both higher and lower molecular exclusions than 65-75 kDa may also be employed to co-purify and thus enrich Albumin with hsps.

Also due to their similar molecular weights, any method that separates polypeptides based on size should effectively co-purify albumin and hsp70 such as molecular sieves and gel filtration or size exclusion chromatography. In addition, due to the similar nature of Hsp70 and Albumin in terms of hydrophobicity and electronegativity or surface charge may be co-purified by precipitation under a number of conditions. Some of those conditions are precipitation by ammonium sulfate, precipitation by denaturants such as urea, or precipitation based on isoelectric point and solubility.

The methods are also applicable to enrich albumin with hsps from other sources. For example albumin derived from native and transgenic animal feedstock serum, as well as albumin produced from recombinant organisms and tissue culture systems based on prokaryotic and eukaryotic cells, including, vertebrate cells such as mammalian cells, and non vertebrate cells, such as insects, as well as plant, and fungi such as yeast, and the like.

### V Exemplary Cells

Without wishing to be bound by theory, it is contemplated that any cell which is susceptible to apoptosis may be used in the methods of the invention, including primary cells, immortalized cells, differentiated cells, undifferentiated cells or cells, such as stem cells, with varying degrees of specialization. In a particular embodiment, cells used in the methods of the invention are transfected with a nucleic acid molecule comprising a nucleotide sequence encoding a protein of interest, e.g., a therapeutic protein or an antibody.

In a particular embodiment, the cells used in the methods of the invention are eukaryotic cells, e.g., mammalian cells. Examples of mammalian cells include, but are not limited to, for example, human B-cells, and T cells, and derivatives thereof, such as hybridomas, and cell expressing markers of B or T cells, monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/-DHFR(CHO, Urlaub and Chasin, Proc. Natl. Acad. Sci. USA, 77:4216 (1980)); CHO-K1 cell (ATCC CCL-61), human PER.C6 cells (Crucell, Nev.), mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; NSO mouse myeloma cells (ECACC; SIGMA), and a human hepatoma line (Hep G2). Additional examples of useful cell lines include, but are not limited to, HT1080 cells (ATCC CCL 121), MCF-7 breast cancer cells (ATCC BTH 22), K-562 leukemia cells (ATCC CCL 243), KB carcinoma cells (ATCC CCL 17), 2780AD ovarian carcinoma cells (see Van der Blick, A. M. et al., Cancer Res. 48:5927-5932 (1988), Raji cells (ATCC CCL 86), Jurkat cells (ATCC TIB 152), Namalwa cells (ATCC CRL 1432), HL-60 cells (ATCC CCL 240), Daudi cells (ATCC CCL 213), RPMI 8226 cells (ATCC CCL 155), U-937 cells (ATCC CRL 1593), Bowes Melanoma cells (ATCC CRL 9607), WI-38VA13 subline 2R4 cells (ATCC CLL 75.1), and MOLT-4 cells (ATCC CRL 1582), as well as heterohybridoma cells produced by fusion of human cells and cells of another species. These and other cells and cell lines are available commercially, for example from the American Type Culture Collection (Virginia, USA). Many other cell lines are known in the art and will be familiar to the ordinarily skilled artisan; such cell lines therefore can be used equally well in the methods of the present invention. In a particular embodiment, cells used in the methods of the invention are CHO cells or NSO cells. Hybridomas and antibody-producing cells may also be used in the methods of the invention.

In another embodiment, cells used in any of the methods of the invention are stem cells. Stem cells are undifferentiated cells defined by their ability at the single cell level to both self-renew and differentiate to produce progeny cells, including self renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Types of human stem cells that may be used in any of the methods of the invention include established lines of human cells derived from tissue formed after gestation, including pre-embryonic tissue (such as, for example, a blastocyst), embryonic tissue, or fetal tissue taken any time during gestation, typically but not necessarily before approximately 10-12 weeks gestation. Non-limiting examples are established lines of human embryonic stem cells or human embryonic germ cells, such as, for example the human embryonic stem cell lines H1, H7, and H9 (WiCell). Also contemplated is use of the compositions of this disclosure during the initial establishment or stabilization of such cells, in which case the source cells would be primary pluripotent cells taken directly from the source tissues. Also, suitable are stem cells isolated from blood or cord blood. Also suitable are cells taken from a pluripotent stem cell population already cultured in the absence of feeder cells. Also suitable are mutant human stem cell lines, such as, for example, BG01v (BresaGen, Athens, Ga.). In one embodiment, Human stem cells are prepared as described by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995).

Additionally, hybridoma cells can also be used in the methods of the invention. The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line which produces antibodies such as, for example, quadromas. See, e.g., Milstein et al., Nature, 537:3053 (1983). The hybrid cell lines can be of any species, including human, rabbit and mouse.

In some embodiments, a cell line used in the methods of the invention is an antibody-producing cell line. Antibody-producing cell lines may be selected and cultured using techniques well known to the skilled artisan. See, e.g., Current Protocols in Immunology, Coligan et al., Eds., Green Publishing Associates and Wiley-Interscience, John Wiley and Sons, New York (1991) which is herein incorporated by reference in its entirety, including supplements. In general, any cell suitable for recombinant protein expression in cell culture can be used in the methods of the invention.

In some embodiments, the cells used in the methods of the present invention may include a heterologous nucleic acid molecule which encodes a desired recombinant protein, e.g., a therapeutic protein or antibody which is desired to be produced using the methods of the invention. In a particular embodiment, the methods of the present invention are useful for producing high titers of a desired recombinant protein, e.g., a therapeutic protein or antibody, in the presence of reduced levels of one or more contaminants.

### VI. Cell Culture Media

Any suitable culture medium or feed medium suitable for cell growth and protein production may be used in the methods of the invention. Suitable culture or feed mediums are chosen for their compatibility with the host cells and process of interest. Suitable culture or feed mediums are well known in the art and include, but are not limited to, commercial media such as Ham's F10 (SIGMA), Minimal Essential Medium (SIGMA), RPMI-1640 (SIGMA), and Dulbecco's Modified Eagle's Medium SIGMA) are suitable for culturing the animal cells. In addition, any of the media described in Ham and Wallace, (1979) Meth. Enz., 58:44; Barnes and Sato, (1980) Anal. Biochem. 102:255; U.S. Pat. No. 4,767,704; 4,657,866; 4,927,762; 5,122,469 or 4,560,655; International Publication Nos. WO 90/03430; and WO 87/00195 may be used.

Any such media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. The necessary growth factors for a particular cell are readily determined empirically without undue experimentation, as described for example in Mammalian Cell Culture (Mather, J. P. ed., Plenum Press, N.Y. (1984), and Barnes and Sato, Cell, 22:649 (1980).

Other methods, vectors, and host cells suitable for adaptation to the synthesis of the protein of interest in recombinant vertebrate cell culture are described in Gething et al., Nature, 293:620-625 (1981); Mantei et al., Nature, 281:40-46 (1979); EP 117,060; and EP 117,058. In general, principles, protocols, and practical techniques for maximizing the productivity of mammalian cell cultures can be found in Mammalian Cell Biotechnology: A Practical Approach, M. Butler, ed. (IRL Press, 1991).

### VII. Exemplary Cell Culture Expression Products

In one aspect of any of the claimed methods, the supplements of the invention are used to improve the viability and growth of a cell which is used to express and produce a protein of interest. The cell may express the protein of interest endogenously or may be an engineered cell line that has been modified genetically to express the protein of interest at levels above background for that cell.

Cells may be genetically modified to express a protein by transformation with a nucleic acid encoding the protein of interest, or by transformation of an activating sequence that promotes the expression of an endogenous gene. In one aspect the protein of interest may be expressed from an expression vector, in which a coding sequence for the protein of interest is operably linked to an expression control sequences, to enable either constitutive or inducible expression, as is known in the art.

The protein of interest may be any protein, or fragment thereof, which is of commercial, therapeutic or diagnostic value including without limitation cytokines, chemokines, hormones, antibodies, anti-oxidant molecules, engineered immunoglobulin-like molecules, a single chain antibodies, a humanized antibodies, fusion proteins, enzymes, immune co-stimulatory molecules, immunomodulatory molecules, transdominant negative mutants of a target protein, toxins, conditional toxins, antigens, a tumor suppresser proteins, growth factors, membrane proteins, vasoactive proteins and peptides, anti-viral proteins and ribozymes, and derivatives thereof (such as with an associated reporter group). The protein of interest may also comprise pro-drug activating enzymes.

In some embodiments, the protein of interest comprises a glycoprotein, or any other protein which has one or more post-translational modifications. For example, any protein which is suitable for production in a eukaryotic host may be expressed using the methods and compositions described here.

The methods of the invention can be used to produce any desired recombinant protein or fragment thereof. In some embodiments, a recombinant protein produced using the methods described herein is a therapeutic protein. In other embodiments, the recombinant protein is an antibody or functional fragment thereof. Antibodies which may be produced using the methods of the invention include, for example, polyclonal, monoclonal, monospecific, polyspecific, fully human, humanized, single-chain, chimeric, hybrid, CDR grafted. It may comprise a full length IgG1 antibody or an antigen-binding fragments thereof, such as, for example, Fab, F(ab′)₂, Fv, and scfv.

Antibodies within the scope of the present invention include, but are not limited to: anti-HER2 antibodies including Trastuzumab (HERCEPTIN™) (Carter et al., Proc. Natl. Acad. Sci. USA, 89:4285-4289 (1992), U.S. Pat. No. 5,725,856); anti-CD20 antibodies such as chimeric anti-CD20“C2B8” as in U.S. Pat. No. 5,736,137 (RITUXAN™), a chimeric or humanized variant of the 2H7 antibody as in U.S. Pat. No. 5,721,108, B1, or Tositumomab (BEXXAR™); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN™. (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58:377-380 (1994)); anti-IgE (Presta et al., J. Immunol. 151:2623-2632 (1993), and International Publication No. WO 95/19181); anti-CD18 (U.S. Pat. No. 5,622,700, issued Apr. 22, 1997, or as in WO 97/26912, published Jul. 31, 1997); anti-IgE (including E25, E26 and E27; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-alpha, antibodies including cA2 (REMICADE™), CDP571 and MAK-195 (See, U.S. Pat. No. 5,672,347 issued Sep. 30, 1997, Lorenz et al. J. Immunol. 156(4):1646-1653 (1996), and Dhainaut et al. Crit. Care Med. 23(9):1461-1469 (1995)); anti-Tissue Factor (TF) (European Patent No. 0 420 937 B1 granted Nov. 9, 1994); anti-human alpha 4 beta 7 integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT™) and (ZENAPAX™) (See U.S. Pat. No. 5,693,762 issued Dec. 2, 1997); anti-CD4 antibodies such as the cM-7412 antibody (Choy et al. Arthritis Rheum 39(1):52-56 (1996)); anti-CD52 antibodies such as CAMPATH-1H (Riechmann et al. Nature 332:323-337 (1988)); anti-Fc receptor antibodies such as the M22 antibody directed against Fc gamma RI as in Graziano et al. J. Immunol. 155(10):4996-5002 (1995); anti-carcinoembryonic antigen (CEA) antibodies such as hMN-14 (Sharkey et al. Cancer Res. 55(23Suppl): 5935s-5945s (1995); antibodies directed against breast epithelial cells including huBrE-3, hu-Mc 3 and CHL6 (Ceriani et al. Cancer Res. 55(23): 5852s-5856s (1995); and Richman et al. Cancer Res. 55(23 Supp): 5916s-5920s (1995)); antibodies that bind to colon carcinoma cells such as C242 (Litton et al. Eur J. Immunol. 26(1):1-9 (1996)); anti-CD38 antibodies, e.g. AT 13/5 (Ellis et al. J. Immunol. 155(2):925-937 (1995)); anti-CD33 antibodies such as Hu M195 (Jurcic et al. Cancer Res 55(23 Suppl):5908s-5910s (1995) and CMA-676 or CDP771; anti-CD22 antibodies such as LL2 or LymphoCide (Juweid et al. Cancer Res 55(23 Suppl):5899s-5907s (1995)); anti-EpCAM antibodies such as 17-1A (PANOREX™); anti-GpIIb/IIIa antibodies such as abciximab or c7E3 Fab (REOPRO™ anti-RSV antibodies such as MEDI-493 (SYNAGIS™); anti-CMV antibodies such as PROTOVIR™; anti-HIV antibodies such as PRO542; anti-hepatitis antibodies such as the anti-Hep B antibody OSTAVIR™; anti-CA 125 antibody OvaRex; anti-idiotypic GD3 epitope antibody BEC2; anti-.alpha.v.beta.3 antibody VITAXIN™; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 and the anti-HLA DR antibody Oncolym (Lym-1). The preferred target antigens for the antibody herein are: HER2 receptor, VEGF, IgE, CD20, CD11a, and CD40.

The recombinant protein may be a cellular protein such as a receptor (e.g., membrane bound or cytosolic) or a structural protein (e.g. a cytoskeleton protein). The recombinant protein may be cellular factor secreted by the cell or used internally in one or more signal transduction pathways. Non limiting examples include, but are not limited to, CD2, CD3, CD4, CD8, CD11a, CD14, CD18, CD20, CD22, CD23, CD25, CD33, CD40, CD44, CD52, CD80 (B7.1), CD86 (B7.2), CD147, IL-1, IL-2, IL-3, IL-7, IL-4, IL-5, IL-8, IL-10, IL-2 receptor, IL-4 receptor, IL-6 receptor, IL-13 receptor, IL-18 receptor subunits, PDGF, EGF receptor, VEGF receptor, hepatocyte growth factor, osteoprotegerin ligand, interferon gamma, B lymphocyte stimulator C5 complement TAG-72, integrin alpha 4 beta 7, the integrin VLA-4, B2 integrins, TRAIL receptors 1, 2, 3, and 4, RANK, RANK ligand, TNF, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2, and CTLA4 (which is a cytotoxic T lymphocyte-associated antigen).

The recombinant protein may also be derived from an infectious agent such as a virus, a bacteria, or fungus. For example, the protein may be derived from a viral coat or may be a viral enzyme or transcription factor. The protein may be derived from a bacterial membrane or cell wall, or may be derived from the bacterial cytosol. The protein may be a yeast enzyme, transcription factor, or structural protein. The yeast protein may be membrane bound, cytsolic, or secreted. Examples of infectious agents include, but are not limited to, respiratory syncitial virus, human immunodeficiency virus (HIV), hepatitis B virus (HBV), hepatitis C virus (HCV), Streptococcus mutans, and Staphlycoccus aureus, and Candida albicans. Moreover, the product of the cell culture system may be a virus such as any of those noted above. These viruses include live viruses, attenuated viruses and otherwise inactivated viruses or components thereof such as viral particles or virus-like-particles. The virus can also be pseudotyped viruses in which the components of the virus are comprised of components of two or more different viruses. In addition, the product of the cell culture can be a vaccine. Vaccines can be therapeutic or prophylactic in nature. Vaccines produced in cultures are often live or attenuated viruses or components thereof as exemplified by subunit vaccines or can be recombinant viruses or virus-like particles comprising components of more than one virus.

The methods of the invention can also be used to produce recombinant fusion proteins comprising all or part of any of the above-mentioned proteins. For example, recombinant fusion proteins comprising one of the above-mentioned proteins plus a multimerization domain, such as a leucine zipper, a coiled coil, an Fc portion of an antibody, or a substantially similar protein, can be produced using the methods of the invention. See e.g. International Application No. WO 94/10308; Lovejoy et al. (1993), Science 259:1288-1293; Harbury et al. (1993), Science 262: 1401-05; Harbury et al. (1994), Nature 371:80-83; Hang. kansson et al. (1999), Structure 7:255-64.

Also encompassed by this invention are pharmaceutical compositions including one or more recombinant proteins produced by the methods described herein. In some embodiments, pharmaceutical compositions further include a pharmaceutically acceptable carrier. The term “pharmaceutically-acceptable carrier” as used herein means one or more compatible solid or liquid filler, diluents or encapsulating substances which are suitable for administration into a subject.

### VIII Stem Cells

In one embodiment, human stem cells are cultured in a culture system that is essentially free of feeder cells, but nonetheless supports proliferation of human embryonic stem cells without undergoing substantial differentiation, comprising a supplement of the invention. The growth of human stem cells in feeder-free culture without differentiation is supported using a medium conditioned by culturing previously with another cell type and further comprising a supplement of the present invention. Alternatively, the growth of human stem cells in feeder-free culture without differentiation is supported using a chemically defined medium comprising a supplement of the present invention. Examples of feeder-free, serum free culture systems in which stem cells are maintained in unconditioned serum replacement (SR) medium supplemented with different growth factors capable of triggering stem cell self-renewal include those disclosed in US patent applications, US20050148070, US20050244962, US20050233446, U.S. Pat. No. 6,800,480, and PCT publications WO2005065354 and WO2005086845.

In an alternate embodiment, human stem cells are initially cultured with a layer of feeder cells that support the human stem cells and further comprising a supplement of the present invention. The human are then transferred to a culture system that is essentially free of feeder cells, but nonetheless supports proliferation of human stem cells without undergoing substantial differentiation and which further comprises a supplement of the present invention. In any of these approaches, the use of the supplements of the invention results in significantly enhanced rates of cell growth and improved cell viability.

Examples of conditioned media suitable for use with the supplements of the present invention are disclosed in US20020072117, U.S. Pat. No. 6,642,048, WO2005014799, and Xu et al (Stem Cells 22: 972-980, 2004). An example of a chemically defined medium suitable for use with the supplements of the present invention may be found in US20070010011.

Examples of feeder cells include feeder cells selected from the group consisting of a fibroblast cell, a MRC-5 cell, an embryonic kidney cell, a mesenchymal cell, an osteosarcoma cell, a keratinocyte, a chondrocyte, a Fallopian ductal epithelial cell, a liver cell, a cardiac cell, a bone marrow stromal cell, a granulosa cell, a skeletal muscle cell, a muscle cell and an aortic endothelial cell. In a preferred embodiment, the MRC-5 cell, has ATCC Catalog Number 55-X; the transformed and has ATCC Accession Number CRL-2309; the human osteosarcoma cell has ATCC Accession Number HTB-96; and the mesenchymal cell is a human fetal palatal mesenchymal cell with ATCC Accession Number CRL-1486. In other preferred embodiments the human fibroblast cell is a skin keloid fibroblast, KEL FIB and has ATCC Accession Number CRL-1762, or is a fetal skin fibroblast cell; and the bone marrow stromal cell, HS-5, has ATCC Accession Number CRL-11882.

Suitable culture media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12/50% DMEM basal medium; 200 mM L-glutamine, Gibco #15039-027; non-essential amino acid solution, Gibco 11140-050; β-mercaptoethanol, Sigma # M7522; human recombinant basic fibroblast growth factor (bFGF), Gibco #13256-029.

In one embodiment, the human stem cells are plated onto a suitable culture substrate that is treated prior to treatment according to the methods of the present invention, with a composition comprising a supplement of the present invention. In one embodiment, the treatment is an extracellular matrix component, such as, for example, those derived from basement membrane or that may form part of adhesion molecule receptor-ligand couplings. In one embodiment, the suitable culture substrate is MATRIGEL (Becton Dickenson). MATRIGEL is a soluble preparation from Engelbreth-Holm-Swarm tumor cells that gels at room temperature to form a reconstituted basement membrane.

Other extracellular matrix components and component mixtures are suitable as an alternative and can be used with the supplements of the present invention. This may include laminin, fibronectin, proteoglycan, entactin, heparan sulfate, and the like, alone or in various combinations with a supplement of the present invention.

In another embodiment, the invention encompasses a stem cell culture, comprising a human pluripotent stem cell and a feeder-free, serum free culture system comprising a supplement of the invention. In one embodiment the invention encompasses a human pluripotent stem cell culture, comprising a human pluripotent stem cell and a feeder-free, serum free culture system comprising a supplement of the invention.

In another embodiment the invention encompasses an stem cell culture, comprising a human stem cell and a human feeder cell culture comprising a supplement of the invention. In another embodiment the invention encompasses a human pluripotent stem cell culture, comprising a human pluripotent stem cell and a human feeder cell culture comprising a supplement of the invention.

In another embodiment, the present invention provides a method for deriving a population of cells comprising cells expressing pluripotency markers, comprising the steps of:
a. Culturing human stem cells,
b. Differentiating the human stem cells into cells expressing pluripotency markers, wherein the differentiation is conducted in the presence of a supplement of the present invention.

In another embodiment, the present invention provides a method for deriving a population of cells comprising cells expressing markers, characteristic of ectodermal, endodermal or mesodermal cells, comprising the steps of:
a. Culturing pluripotency stem cells;
b. Differentiating the pluripotency stem cells into cells expressing markers characteristic of ectodermal, endodermal or mesodermal cells, wherein the differentiation is conducted in the presence of a supplement of the present invention.

In any of these methods, the stem cells can be differentiated into cells expressing markers characteristic of an endodermal, ectodermal or mesodermal lineage by any method in the art. For example, cells expressing pluripotency markers may be differentiated into cells expressing markers characteristic of the definitive endoderm lineage according to the methods disclosed in D'Amour et al, Nature Biotechnology 23, 1534-1541 (2005), by Shinozaki et al, Development 131, 1651-1662 (2004), McLean et al., Stem Cells 25, 29-38 (2007), D'Amour et al., Nature Biotechnology 24, 1392-1401 (2006).

Cells expressing markers characteristic of the endoderm lineage may be further differentiated into cells expressing markers characteristic of the pancreatic endocrine lineage by any method in the art. For example, cells expressing markers characteristic of the pancreatic endoderm lineage may be differentiated into cells expressing markers characteristic of the pancreatic endocrine lineage according to the methods disclosed in D'Amour et al, Nature Biotechnology 24, 1392-1401 (2006), wherein the differentiation is conducted in the presence of a supplement of the present invention.

In one aspect of any of these methods of differentiation, the human stem cells are cultured and differentiated on a tissue culture substrate coated with an extracellular matrix. The extracellular matrix may be a solubilized basement membrane preparation extracted from mouse sarcoma cells (which is sold by BD Biosciences under the trade name MATRIGEL). Alternatively, the extracellular matrix may be growth factor-reduced MATRIGEL. Alternatively, the extracellular matrix may be fibronectin. In an alternate embodiment, the human stem cells are cultured and differentiated on tissue culture substrate coated with human serum. In one aspect, the tissue culture substrate is coated with extracellular matrix and a supplement of the present invention.

The extracellular matrix may be diluted prior to coating the tissue culture substrate. Examples of suitable methods for diluting the extracellular matrix and for coating the tissue culture substrate may be found in Kleinman, H. K., et al., Biochemistry 25:312 (1986), and Hadley, M. A., et al., J. Cell. Biol. 101:1511 (1985).

In one aspect of the methods of stem cell differentiation, the culture medium should contain sufficiently low concentrations of certain factors to allow the differentiation of human stem cells to cells of endoderm, ectoderm or mesoderm lineage, such as, for example insulin and IGF (as disclosed in WO2006020919). This may be achieved by lowering the serum concentration, or alternatively, by using chemically defined media that lacks insulin and IGF. Examples of chemically defined media are disclosed in Wiles et al (Exp Cell Res. 1999 Feb. 25; 247(1): 241-8.). In a preferred embodiment, of any of these methods, the culture media comprises a supplement of the present invention.

The culture medium may also contain at least one other additional factor that may enhance the formation of cells expressing markers characteristic of endoderm, mesoderm or ectoderm lineage from human stem cells. The at least one additional factor may be, for example, nicotinamide, members of TGF-β family, including TGF-β1, 2, and 3, serum albumin, members of the fibroblast growth factor family, platelet-derived growth factor-AA, and -BB, platelet rich plasma, insulin growth factor (IGF-I, II), growth differentiation factor (GDF-5, -6, -8, -10, 11), glucagon like peptide-I and II (GLP-I and II), GLP-1 and GLP-2 mimetobody, Exendin-4, retinoic acid, parathyroid hormone, insulin, progesterone, aprotinin, hydrocortisone, ethanolamine, beta mercaptoethanol, epidermal growth factor (EGF), gastrin I and II, copper chelators such as, for example, triethylene pentamine, forskolin, Na-Butyrate, activin, betacellulin, ITS, noggin, neurite growth factor, nodal, valproic acid, trichostatin A, sodium butyrate, hepatocyte growth factor (HGF), sphingosine 1, VEGF, MG132 (EMD, CA), N2 and B27 supplements (Gibco, Calif.), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, or combinations thereof. In a preferred embodiment, of any of these methods, the culture media containing at least one additional factor listed above, further comprises a supplement of the present invention.

The at least one other additional factor may be supplied by conditioned media obtained from pancreatic cells lines such as, for example, PANC-1 (ATCC No: CRL-1469), CAPAN-1 (ATCC No: HTB-79), BxPC-3 (ATCC No: CRL-1687), HPAF-II (ATCC No: CRL-1997), hepatic cell lines such as, for example, HepG2 (ATCC No: HTB-8065), and intestinal cell lines such as, for example, FHs 74 (ATCC No: CCL-241). In a preferred embodiment, of any of these methods, the conditioned media further comprises a supplement of the present invention.

In another embodiment, the invention encompasses a method of using the cell or tissue of any of the aforementioned stem cells for the experimental, therapeutic and prophylactic treatment of a disease or condition in a human or animal. Preferably, the disease is selected from the group consisting of Parkinson's, Alzheimer's, Multiple Sclerosis, spinal cord injuries, stroke, macular degeneration, burns, liver failure, heart disease, diabetes, Duchenne's muscular dystrophy, osteogenesis imperfecta, osteoarthritis, rheumatoid arthritis, anemia, leukemia, breast cancer, solid tumors, and AIDS. In a preferred embodiment, the disease is Parkinson's or Alzheimer's. In a more preferred embodiment, the disease is Parkinson's.

### IX. Large Scale Production of Recombinant Proteins

In one embodiment, the supplements of the present invention can be used to produce a protein of interest by growing host cells in the presence of the supplement. In one embodiment, the cell culture is performed in a stirred tank bioreactor system and a fed batch culture procedure is employed. In another embodiment a wave disposable bioreactor is employed. In the bioreactor system, the size of the bioreactors are sufficiently large to produce the desired amount of protein of interest, such as 1,000 Liter or 12,000 Liter sizes, but are not limited to such sizes as much smaller (i.e., 2 Liter, 400 Liter) or larger (i.e., 25,000 Liter, 50,000 Liter) bioreactor vessels may be appropriate. In the preferred fed batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process but at the termination of the culture process (in perfusion culturing, the cells are restrained in the culture by, e.g., filtration, encapsulation, anchoring to microcarriers etc. and the culture medium is continuously or intermittently introduced and removed from the culturing vessel).

Further, the cultured cells may be propagated according to any scheme or routine that may be suitable for the particular host cell and the particular production plan contemplated. Therefore, the present invention contemplates a single step or multiple step culture procedure. In a single step culture, the host cells are inoculated into a culture environment and the method steps of the instant invention are employed during a single production phase of the cell culture. Alternatively, a multi-stage culture is envisioned. In the multi-stage culture, cells may be cultivated in a number of steps or phases. For instance, cells may be grown in a first step or growth phase culture wherein cells, possibly removed from storage, are inoculated into a medium comprising a supplement of the present invention suitable for promoting growth and high viability. The cells may be maintained in the growth phase for a suitable period of time by the addition of fresh medium to the host cell culture.

According to a preferred aspect of the invention, fed batch or continuous cell culture conditions are devised to enhance growth of the mammalian cells in the growth phase of the cell culture. In the growth phase, cells are grown under conditions and for a period of time that is maximized for growth. Culture conditions, such as temperature, pH, dissolved oxygen (dO₂) and the like, are those used with the particular host and will be apparent to the ordinarily skilled artisan. Generally, the pH is adjusted to a level between about 6.5 and 7.5 using either an acid (e.g., CO₂) or a base (e.g., Na₂CO₃ or NaOH). A suitable temperature range for culturing mammalian cells such as CHO cells is between about 30 to 38° C. and preferably about 37° C. and a suitable dO₂ is between 5-90% of air saturation.

At a particular stage the cells may be used to inoculate a production phase or step of the cell culture. Alternatively, as described above, the production phase or step may be continuous with the inoculation or growth phase or step.

According to the present invention, the cell culture environment during the production phase of the cell culture is controlled. According to the steps of the presently disclosed methods, the addition of the supplements of the invention can be coordinated such that the desired content and quality of the protein of interest is achieved and maintained in the resulting cell culture fluid. In a preferred aspect, the production phase of the cell culture is preceded by a transition phase of the cell culture in which the addition of the supplements of the invention initiates the production phase of the cell culture.

In any of the above-described methods, it is contemplated that it may be desirable to include a desired amount of agent like butyrate or Trichostatin A in the cell culture medium in combination with a supplement of the invention. Various forms of butyrate and its salts are known in the art, such as butyric acid and sodium butyrate, and are publicly available from sources such as Sigma Chemical Co. Butyrate has been reported in the literature to enhance the productivity and protein expression of cell cultures [Arts et al., Biochem J., 310:171-176 (1995); Gorman et al., Nucleic Acids Res., 11:7631-7648 (1983); Krugh, Mol. Cell. Biochem., 42:65-82 (1982); Lamotte et al., Cytotechnology, 29:55-64 (1999); Chotigeat et al., Cytotechnology, 15:217-221 (1994)]. Trichostatin A (TSA) is an inhibitor of histone deacetylase and may act similarly to butyrate in enhancing the productivity and protein expression in cell cultures [Medina et al., Cancer Research, 57:3697-3707 (1997)]. Although butyrate has some positive effects on protein expression, it is also appreciated in the art that at certain concentrations, butyrate can induce apoptosis in the cultured cells and thereby decrease viability of the culture as well as viable cell density [Hague et al., Int. J. Cancer, 55:498-505 (1993); Calabresse et al., Biochim. Biophys. Res. Comm., 195:31-38 (1993); Fillipovich et al., Biochim. Biophys. Res. Comm., 198:257-265 (1994); Medina et al., Cancer Research, 57:3697-3707 (1997)]. In the methods of the present invention, a desired amount of butyrate or TSA may be added to the cell culture at the onset of the production phase and more preferably, may be added to the cell culture after a temperature shift has been implemented. Butyrate or TSA can be added in a desired amount determined empirically by those skilled in the art, but preferably, butyrate is added to the cell culture at a concentration of about 1 to about 25 mM, and more preferably, at a concentration of about 1 to about 6 mM.

Expression of the protein of interest may be measured in a sample directly, for example, by ELISA, conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe. Various labels may be employed, most commonly radioisotopes, and particularly ³²P. However, other techniques may also be employed, such as using biotin-modified nucleotides for introduction into a polynucleotide. The biotin then serves as the site for binding to avidin or antibodies, which may be labeled with a wide variety of labels, such as radionucleotides, fluorophors or enzymes. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like.

Antibodies useful for immunohistochemical staining and/or assay of sample fluids may be either monoclonal or polyclonal, and may be prepared in any mammal. Many are commercially available.

The supplements claimed herein can also be used to increase transfection efficiency and viability of cells during transfection. Conditions and reagents used in various transfection techniques, such as Lipofectamine are relatively toxic to the cells, while electroporation can severely stress a cell. The use of higher concentrations of transfection reagents, and more extensive electroporation conditions is preferred to achieve higher transfection efficiencies. Thus the addition of the supplements of the invention prior, with, and after transfection can result in higher transfection efficiencies, and higher yields of recombinant proteins.

The supplements of the invention can be used to express proteins of interest which induce apoptosis, such as Apo-2 ligand/TRAIL or Fas ligand. The presence of the supplements of the invention may block such apoptotic activity and allow for improved expression of the protein of interest.

In addition, the methods can be used to increase the viability of cells undergoing freezing/storage/thawing procedures. During these procedures generally cells can lose viability. The presence of apoptosis inhibitors added to the cell culture media can provide for increased cell viability and aid in reducing or eliminating the variability in cell viabilities between aliquots or vials of cells.

### X. Kits

Also encompassed by the present invention are kits for promoting the viability of cells. In one embodiment, a kit according to the present invention comprises: (a) one or more reagents or devices for transfection and (b) a supplement of the present invention. In some embodiments, kits featured herein include instructions and/or promotional materials including details regarding using the transfection device, transfection agent and supplement.

In another embodiment a kit according to the present invention comprises: (a) one or more reagents or devices for freezing or thawing cells and (b) a supplement of the present invention. In some embodiments, kits featured herein include instructions and/or promotional materials including details regarding protocols for freezing or thawing cell lines and the use of the reagents.

In another embodiment a kit according to the present invention comprises: (a) one or more tissue culture products for culturing cells and (b) a supplement of the present invention. In some embodiments, kits featured herein include instructions and/or promotional materials including details regarding protocols for dilution cloning techniques and the use of the reagents in such approaches.

### EXAMPLES

### Example 1

### Production of Recombinant Rice Flour

Methods:

Protein sequences of human serum albumin from various data bases were compared. The consensus sequence represented by accession number P02768 was used as base for gene codon-optimization for suitable expression of human serum albumin in rice grain as described previously in WO2007/002762. Gene synthesis was carried out by Blue Heron (Seattle, Wash.) and the synthetic fragment was inserted into a pUC based vector to create pUC-HSA. After confirmation of the correct DNA sequences, the vector was digested with Mly1 and Xho1. The fragment containing the codon-optimized HSA gene was inserted into pAP1405, which had been precut with Nae1 and Xho1. Plasmid AP1405 was a derivate of vector pAP1441 (WO2007/002762) which includes a Gt1 promoter, Gt1 signal sequence and a nos terminator. Insertion of Mly/Xho 1 fragment into pAP1405 resulted in vector pAP1504 which was used for transfection by bombardment as described below.

The basic procedures of particle bombardment-mediated rice transformation and plant regeneration were carried out as described previously (Yi Chuan Xue Bao. 2001; 28(11):1012-8. Chinese and Biotechnol Prog. 2001; 17(1):126-33). Rice variety TP309 seeds were dehusked, sterilized in 50% (v/v) commercial bleach for 25 min and washed with sterile water. The sterilized seeds were placed on rice callus induction medium (RCI) plates containing [N6 salts (Sigma), B5 vitamins (Sigma), 2 mg/l 2,4-D and 3% sucrose]. The rice seeds were incubated for 10 days to induce callus formation. Primary callus was dissected from the seeds and placed on RCI for 3 weeks. This was done twice more to generate secondary and tertiary callus which was used for bombardment and continued subculture. A callus of 1-4 mm diameter was placed in a 4 cm circle on RCI with 0.3M mannitol 0.3M sorbitol for 5-24 hrs prior to bombardment. Microprojectile bombardment was carried out using the Biolistic PDC-1000/He system (Bio-Rad). The procedure requires 1.5 mg gold particles (60 ug/ml) coated with 2.5 ug DNA. DNA-coated gold particles were bombarded into rice calli with a He pressure of 1100 psi.

After bombardment, the callus was allowed to recover for 48 hrs and then transferred to RCI with 30 mg/l hygromycin B for selection and incubated in the dark for 45 days at 26.degree. C. Transformed calli were selected and transferred to RCI (minus 2,4-D) containing 5 mg/l ABA, 2 mg/l BAP, 1 mg/l NAA and 30 mg/l hygromycin B for 9-12 days. Transformed calli were transferred to regeneration medium consisting of RCI (minus 2,4-D), 3 mg/l BAP, and 0.5 mg/l NAA without hygromycin B and cultured under continuous lighting conditions for 2-4 weeks. Regenerated plantlets (1-3 cm high) were transferred to rooting medium whose concentration was half that of MS medium (Sigma) plus 1% sucrose and 0.05 mg/l NM. After 2 weeks on rooting medium, the plantlets developed roots and the shoots grew to about 10 cm. The plants were transferred to a 6.5.times.6.5 cm pots containing a mix of 50% commercial soil (Sunshine #1) and 50% soil from rice fields. The plants were covered by a plastic container to maintain nearly 100% humidity and grown under continuous light for 1 week. The transparent plastic cover was slowly shifted over a 1 day period to gradually reduce humidity and water and fertilizers added as necessary. When the transgenic R0 plants were approximately 20 cm in height, they were transferred to a greenhouse where they grew to maturity.

Individual R1 seed grains from the individual R0 regenerated plants were dissected into embryos and endosperms. Expression levels-of recombinant albumin in the isolated rice endosperms were determined. Embryos from the individual R1 grains with high recombinant protein expression were sterilized in 50% bleach for 25 min and washed with sterile distilled water. Sterilized embryos were placed in a tissue culture tube containing ½ MS basal salts with the addition of 1% sucrose and 0.05 mg/l NAA. Embryos were germinated and plantlets having about 7 cm shoots and healthy root systems were obtained in about 2 weeks. Mature R1 plants were obtained as regenerants.

Transgenic rice containing heterologous polypeptides can be converted to rice extracts by either a dry milling or wet milling process. In the dry milling process, transgenic rice seeds containing the heterologous polypeptides are dehusked with a dehusker. The dehusked rice was then ground into a fine flour though a dry milling process, for example, in one experiment, at speed 3 of a model 91 Kitchen Mill from K-TEC.

### Example 2

### Purification of Recombinant Albumin (B0000C)

Methods:

For Ventria grown rice, the rice was harvested by combine or by hand. During this process the mature seeds were separated from the vegetative plant matter by the combine separator or by manual labor. The harvested rice was dried to approximately 12% moisture at which point it is suitable for storage in a clean grain bin, storage tote, supersack, or other container that will protect the grain from birds, rodents, lizards, insects and other pests. When the rice grain is needed for flour, it is first dehusked or dehulled. This process is done under vacuum such that debris and the outer part of the seed are swept away from the endosperm and germ or bran layer. The dehusked grain is then either washed and dried, or washed and processed directly as in wet homogenization, or processed further in the dry, dehusked state. The dry, dehusked material may be debranned by a rice polishing or debranning machine which are common to white rice producers.

Debranned, dehusked rice may be washed at this point and wet-milled or dried for dry milling or processed directly by grinding into flour. Milling with the least amount of shear and heat is preferred as such with a roller mill or pin mill. A hammermill is also suitable. The flour should be ground such that the protein can be extracted to 90% in less than 5 minutes in water with hard agitation. Normally that requires a size of particle that is smaller than 400 micrometers or 4 mm. However, larger particles can be extracted if given longer time. Alternatively, the grain can be washed and wet milled with a liquid homogenizer set up such that 90% of the extractable protein is solubilized.

The flour slurry is typically mixed at a ratio of at least 3 parts water to 1 part flour and up to 20 parts water to 1 part flour. The water typically contains suitable buffers such as Tris/HCl, Citrate, Phosphate, HEPES, or the like, such that the pH is maintained around pH 7 and a small amount of salt such as 100 mM NaCl. After the slurry is homogenized in the case of wet milling, or mixed thoroughly for dry flour, the bulk solids are removed from the slurry by way of solid liquid separation. This is carried out by decanting, centrifugation, or filtration; for example using plate and frame with pads, pressure filter, belt filter, vacuum flask, hydroclone, or vacuum belt filter. After filtration, the compressed cake should be washed with extraction buffer to recover protein from the cake. The addition of diatomateous earth or other filter media is useful in promoting the clarity of the filtrate but is not necessary given the right equipment. Alternatively, a flocculating agent may be used to aid in clarification. The clarified filtrate should be checked for its albumin content and verified that the recovery is consistent with the determined expression level in the rice seed.

In order to remove starches, precipitable proteins, viruses, and other contaminants, 5 M acetic acid is added to the clarified filtrate until the pH reaches 5.0 and the solution turns white. The white solution is agitated for at least 20 minutes to encourage precipitation of insoluble materials. The precipitated solution is then filtered through a depth filter, such as a canister filter, cartridge filter or other filtration device to reach clarity that is suitable for ultrafiltration, or less that 10 NTU (nephelometry turbidity units). It can also be clarified with a filter press, pressure filter, or alternatively by using a ceramic filter or other material that utilizes cross-flow. In addition, this material is suitable for direct application to an expanded bed chromatography column.

In a preferred method, the clarified filtrate is clarified via filtration through a 0.2 micron filter, and neutralized to pH 7.0 with 1M NaOH. This material is then suitable for ultrafiltration by hollow fiber, flat sheet, or spiral wound cross flow filtration. The material can be passed through a membrane of 100 kilodalton (kDa) size or larger to remove viruses, unwanted larger contaminants, and aggregates. The material that passes through the membrane can be concentrated by a 10 or 30 kDa crossflow membrane and then the same membrane can be used to prepare the solution for chromatography. The concentrated material can then diafiltered with column equilibration buffer until the conductivity and the pH are equalized.

The preferred buffer for anion exchange chromatography on GE DEAE Sepharose or GE Q Sepharose is 10 or 20 mM Tris/HCl buffer pH balanced to pH 8.0. In contrast, the preferred buffer for cation exchange, for example via the use of for negatively charged resins or negatively charged resins mixed with a hydrophobic linker (mixed mode absorbents), or alternatively blue Cibicron such Blue Sepharose (GE) is acetate or citrate buffer pH balanced to 4.8 to 5.0

For either system the albumin and other similarly charged proteins will be retained by the matrix and washing is conducted to remove loosely bound material by washing with at least 5 column volumes of loading buffer, which may also include detergents as deemed necessary to help remove hydrophobic impurities. The material can be eluted by charging the column with the same or modified buffers with the pH increased 2-4 units for cation exchange or decreased 2-4 units for anion exchange. The resulting change in pH will allow for the exchange of ions and the protein will be eluted in a sharp band. To increase the purity of the elution fraction, the elution peak can be scrutinized such that the first portion (10%) or last (10%) or both portions can be excluded from the main elution peak. In the preferred method, a solution containing phosphate at 100 mM and pH adjusted to pH 4.0 including 10 mM NaCl is used to elute the protein from GE Q Sepharose (Fast Flow). In this instance, pH and conductivity are used to elute the material allowing the discrimination between non-binding contaminants (flow through and wash) and tighter binding contaminants (those that are retained on the column in 100 mM Phosphate, 10 mM NaCl, and pH adjusted to 4.0).

After elution, if the pH of the eluted material has a pH of less than 6.0, then it is neutralized with 1M NaOH. The resulting solution is then diafiltered against the same buffer for the next chromatography step, which in a preferred method involves flowing the elutent through a column of the same matrix (i.e. Q Sepharose) except in the non-binding mode with 100 mM Phosphate, 10 mM NaCl, and pH 7.0.

The second column step uses the same principles as the first but in reverse mode such that the contaminants that were co-eluted on the binding column have an opportunity to be retained on the matrix at a neutral pH. The flow through material from the first capture column can also be treated with a variety of alternative types of chromatography approaches, for example, cation exchange, hydrophobic, mixed mode, or gel filtration chromatography.

In a preferred method, the flow through material from the Q Sepharose non-binding column is concentrated on a 10 kDa or 30 kDa crossflow membrane until the concentration is between 15 and 25% albumin. The buffer is then changed by diafiltration into a suitable buffer for cell culture such as Dulbeccos PBS or alternatively 20 mM Phosphate, 50 mM NaCl, and pH 7.0. The material is then sterile grade filtered into a sterile container. The sterile filtered material may be treated with detergent to destroy enveloped viruses and to aid in the removal of hydrophobic toxins and contaminants. In a preferred method, 0.5% v/v Triton X-114 or X-100 is added to the 15 to 25% albumin solution at room temperature (less than 23 C and greater than 18 C) and the solution is agitated or stirred for at least hour. The material is then passed over a hydrophobic resin with a molecular weight exclusion limit that is much less than the molecular weight of albumin. Many commercially available resins are available including those from Biorad and Pall Corporation.

The material that is passed over the column may then be tested in cells that are sensitive to detergent to confirm biological activity. The residual detergent that remains should typically be less than 0.005% with respect to the albumin solution. The detergent free flow through can then be sterile filtered into containers for direct shipment, or can have stabilizers added, or can be subjected to pasteurization with stabilizers, or can have stabilizers added before drying or dried directly. The material may be dried by lyophilization or spray drying. Prior to drying, in some instances, it may be useful to subject the material to a virus filtration step using a disposable, validated, virus removing capsule such as is available from GE, Pall, and Millipore. It is common in the art to understand that a pre-filtration step may be necessary in order to effectively and economically pass the concentrated material through a 20 nm filter.

Results:

Rice flour was extracted at 1:5 ratio in phosphate buffered saline and mixed for 20 minutes. The liquid was clarified using a Nalgene filter flask. The subsequent clarified extract was subjected to acid precipitation as is described in the methods. The solution was then filtered and neutralized to give a clarified filtrate. This material was diafiltered against 50 mM Tris/Cl pH 8.0 until the material and buffer were equilibrated. The material was then loaded (300-600 cmh) on a pre-equilibrated GE Q-Sepharose column to allow for 50 g/L binding capacity. The loaded material was washed with the same buffer and the material was then eluted with 100 mM Phosphate, 10 mM NaCl, and pH 4.0 as described above. The material eluted in a sharp peak and the collected eluate had a stable pH of about 5.8. Albumin produced using this method was compared to other sources of Albumin as more fully disclosed below: The eluate was collected in a pool and 1M NaOH was added until the pH was greater than 6.0. The material was then concentrated on a 10 kDa regenerated cellulose membrane approximately 5 fold and approximately five equal volume diafiltrations were carried out with 100 mM phosphate, 10 mM NaCl, pH 7.0. The final diafiltered material was checked for albumin protein content (in relation to the expression level in the starting material should be greater than 80%) and endotoxin level (should be less than 100 EU/mg depending on the feed material). This material was passed (60-160 cmh) over a Q-Sepharose column, equilibrated with 100 mM phosphate, 10 mM NaCl, pH 7.0, of sufficient size to allow for approximately 2-3 times loading volume. The material was washed through the resin with the same buffer and collected. The collected material was diafiltered on a 10 kDa regenerated cellulose membrane and concentrated approximately 10 fold or until the albumin concentration reaches at least 10% or not more than 20% and five equal volume diafiltrations were performed with 20 mM phosphate, 50 mM NaCl, pH 7.0. After sterile grade filtration (0.2 μm), the solution was agitated for 1 hour with 0.5% (v/v) Triton X-100 at 20+/−2° C. After the incubation, the material was passed through Pall SDR resin according to the manufacturer's directions. The flow through material was sterile grade filtered into sterile containers and refrigerated or freeze dried as is common for protein and salt solutions.

### Example 3

### Comparison of Recombinant Albumin Produced from Rice Using B0000C Process Compared to Other Sources of Albumin and Previous Methods for the Production of Albumin

Methods:

Albumin prepared using the method described in Example 2, was compared to albumin prepared using an alternative process (B000) which was previously used to prepare Cellastim (Batches B202 to B217).

### Albumin Production (Old Process, B000):

Rice flour and 25 mM Sodium phosphate, 50 mM Sodium Chloride, was pH balanced to 6.5 with NaOH and mixed for 20 minutes at room temperature with a S/L ratio of approximately 1:10. Filter aid (Cellpure 300) was added at 10 g/L and the slurry was filtered by filter press, vacuum filtration, or centrifugation. The clarified filtrate was acid precipitated to pH 5.0 with 1 M acetic acid. The resulting solution was filtered as described above with the addition of 5 g/L filter aid (Cellpure 300). The material was neutralized immediately to pH 6.5 to 7.0 with 1M NaOH. The material was diafiltered (10 kDa regenerated cellulose for all UFDF steps) with 5 equal diavolumes of the same buffer used for extraction. The material was loaded on a pre equilibrated Q-Sepharose column (GE Healthcare) to allow for 8 g albumin binding per liter of resin at 60 cmh.

After washing the column with 5 column volumes of the same buffer, the albumin was eluted by increasing the salt concentration to 250 mM NaCl in one step. The resulting material was diafiltered against 100 mM Sodium Phosphate, 10 mM NaCl, pH 7.0 with 5-7 equal diavolumes. The resulting material was passed over a Q-Sepharose column equilibrated with the 100 mM Sodium Phosphate, 10 mM NaCl, pH 7.0, and collected as flow-through. The flow-through material was then concentrated and diafiltered against 20 mM sodium phosphate, 10 mM NaCl, pH 7.0 with 5 diavolumes. The final concentrated material was sterile filtered and incubated with 10 g/L of the detergent CHAPS ((3-Cholamidopropyl)dimethylammonio)-1-Propanesulfonic Acid) and mixed at room temperature for 1 hour. After the one hour incubation, the material was passed over a Biorad SM-2 column. The material was sterile filtered and freeze dried.

### Size Exclusion Chromatography Analysis.

Purity analysis by HPLC was carried out in 100 mM phosphate, pH 7.0 on a GF-250 column (Agilent Technologies) at a flow rate of 1 ml/min with the detector set at 214 and 280 nm. A standard curve was developed by injecting 5 different dilutions made by dry powder with a correction factor of 0.92 for salt and moisture. The main peak from 214 nm was integrated either by retention time or alternatively baseline. The unknown sample was injected at a concentration that is within the range of the standard injections. The unknown concentration of albumin per dry powder weight (purity) was calculated from the standard curve. In a typical experiment, the 0, 5, 8, 10, 15, and 20 μg of the standard was injected followed by approximately 10 μg of unknown sample in approximately 50 μL injection volume. The correlation coefficient for the standard curve after integrating the peaks was typically above 0.98.

SDS PAGE and Densitometry:

(Sodium Dodecyl Sulfate Polyacrylamide Gel Electrophoresis).

Samples were prepared by diluting the protein solutions to 1-2 mg/ml to enable a defined amount of each protein to be loaded on to each well. The sample was mixed 1:1 with Tris-Glycine SDS sample buffer (LC2673 Novex) containing reducing agent (Invitrogen NP0004) and heated to 70° C. for 5 minutes. The sample was loaded (10, 20, or 30 μg) onto a Novex 4-20% precast gel and separated at constant voltage (130V) in standard Tris-Glycine-SDS running buffer. The electrophoresis was ended when the tracking dye reached the end of the gel. A molecular weight marker was included in the first lane as a reference.

The gel was stained with G Bioscience (786-35G) and destained with water. A digital image was obtained with a Hewlett Packard Scanner (G4010). The image file was then opened with UN-SCAN-IT (Silk Scientific Corp.). The densitometry was carried out with positive image analysis in 256 grayscale in which all visible bands were included as individual segments. The background noise was corrected by four corner interpolation as specified in the software for each segment. The signal for each segment or band was then calculated from the product of the # of pixels and the average pixel intensity (0-255). The sum of the signals for an entire lane (all visible segments or bands) was taken as 100% and the impurity bands were subtracted to calculate the albumin purity. The percent of each contaminating protein in each band was calculated as the number of peptides identified for that contaminant protein as determined by peptide mapping divided by the total number of all peptides identified in a particular band. The image analysis was repeated 3 times such that the standard deviation is less than 0.5% out of 100%.

### Determination of Endotoxin by the Pyrogene rFC Method.

Endotoxin content was determined by the Pyrogene rFC method. Lyophilized endotoxin standard was mixed with endotoxin free water as specified by the manufacturer (Lonza) to develop a standard curve. The protein samples were either diluted as is for liquid or alternatively, reconstituted with endotoxin free water for powder. Different dilutions were prepared such that the readings should appear within the range of the standard curve. The samples were heated to 100° C. for 10 minutes to dissociate unwanted molecular interactions. In a typical experiment, the sample and standard were added at 100 μl per well, with 0, 0.001, 0.005, 0.01, 0.05, and 0.1 endotoxin units per well. The samples were also added at 100 μl and extra samples were included such that spiking with 0.001-0.01 endotoxin units per well were added to test for assay inhibition or interference. The working reagent was prepared according to the manufacturer (Lonza) by mixing the rFC enzyme, assay buffer, and substrate in a 1:4:5 ratio, respectively. The working reagent was added to the wells at equal volume to the sample or standards. The fluorescence plate reader (Biotek FLX 800T) was set for excitation at 380 nm (bandwidth=20 nm) and the emission wavelength was set at 440 nm (bandwidth=30 nm). The reading taken at time zero is subtracted from the reading taken after 1 hour at 37° C. The readings were considered valid if the correlation coefficient, slope, and Y-intercept for the standards was within the set limits, and the spiking experiments show that the spiked endotoxin was measureable and recoverable within the set limits. In addition, the standard deviation for duplicate samples should be in reasonable agreement such that the standard deviation was within a specified arbitrarily chosen limit. All samples were collected aseptically and the tubes/vials/containers used for testing were verified to be extremely low endotoxin following good laboratory practices as they relate to accurate and precise endotoxin testing.

### Determination of Cell Viability

The hybridoma cell line AE1 (ATCC) was maintained in DMEM basic media containing 5% fetal bovine serum (FBS). Albumin was tested under serum-free conditions (AFM6, KC Bio, Kansas) without supplementation of fetal bovine serum. The cells were subcultured from 5% FBS to serum free media over multiple passages. At each subculture, the cells were analyzed for total cell count and viability in the presence of the indicated concentrations of albumin. (As assessed by trypsinization and direct counting using a Neubauer haemocytometer). The cells were grown under standard culture conditions (5% CO₂ and 37° C.) for approximately 70 hours after which the viability for the cultures was measured. The experiments were conducted in duplicate. Date show the number of viable cells/ml divided by 10⁵.

### Determination of Detergent

The detergent concentration for the albumin was determined by a detergent (cell based) assay. Briefly, detergent sensitive cells were spiked with different amounts of detergent and the resulting cell viability cell determination used to generate a standard curve consisting of 16 independent data points. The change in viability with respect to the change in detergent concentration was plotted and fitted with a logarithmic function. This equation was then used to calculate the unknown detergent concentrations in samples tested in the same cell based assay. The correlation coefficient for the standard curve for the data given was 0.9816. Typically detergent concentrations of greater than about 10 ppm per Cellastim dry weight, result in noticeable toxic activity. By comparison in a 10% albumin solution, toxic effects of detergent become apparent when the detergent concentration is above about 100 ppm to 200 ppm or 0.01% to 0.02% (v/v).

### Results & Discussion:

### I. Analysis by Size Exclusion Chromatography of Plasma Derived Serum (Sigma Albumin), and Recombinant HSA (Cellastim) Produced Using the Process of Example 2 (Cellastim P0171) And the Old Process (Cellastim P0107).

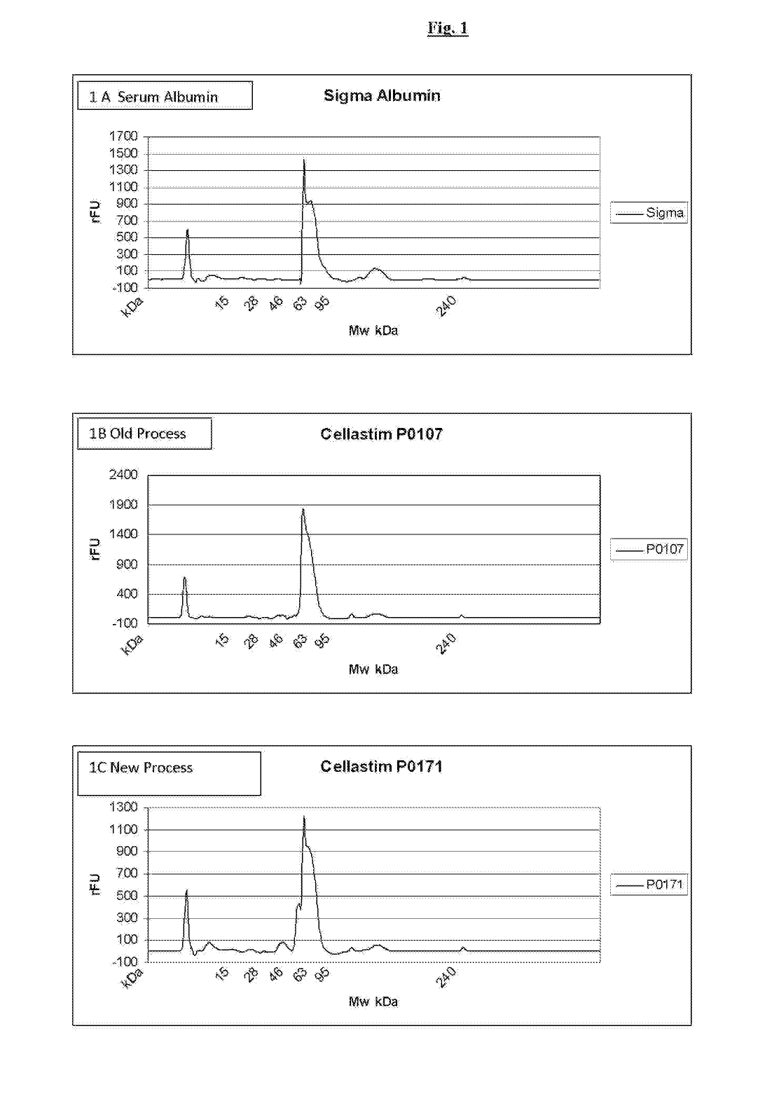

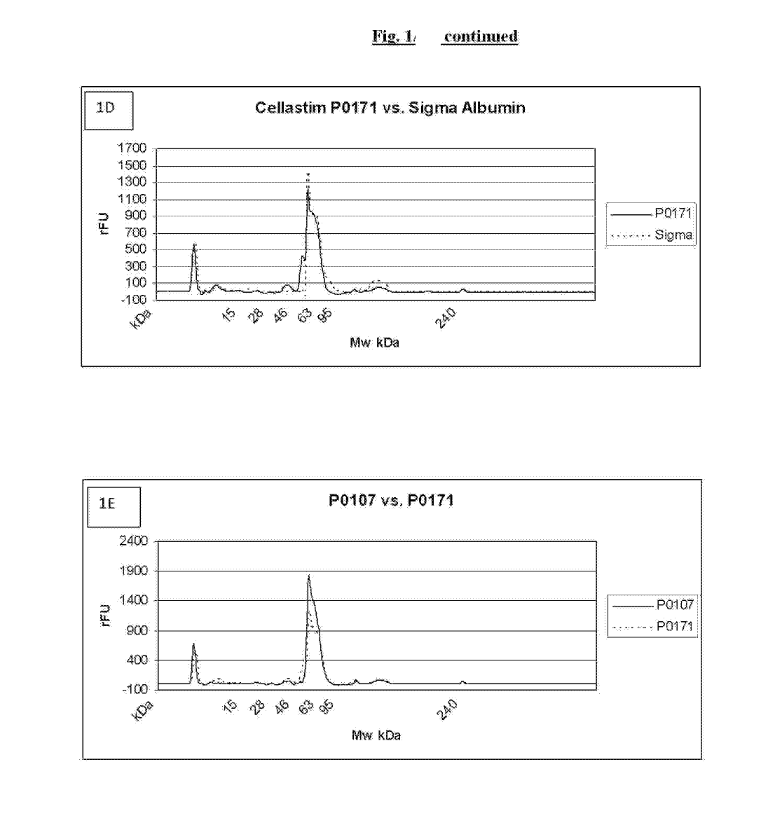

The HPLC size exclusion profiles (FIGS. 1A, C & D) for the three types of albumin show that in terms of overall purity the different albumin preparations are generally similar. Specifically, the peaks at around 4.5 kDa and 240 kDa are the internal controls, while all three products contain a very small amount of an off main peak signal at about 10-12 kDa.

While the human serum derived albumin (Sigma Albumin)(FIG. 1A), contains a contaminant at around 17 kDa, the recombinant rice derived albumin using the new process (Cellastim P0171)(FIG. 1C) contains two protein contaminants of around 44 KDa and 55 kDa that occur in Cellastim made with the new process at significantly higher levels than when using the old process (Cellastim P0107)(FIG. 1B). These peaks are not completely resolved in the HPLC separations, but can be seen as more clearly in the overlaid profiles of Cellastim P0171 and Sigma albumin (FIG. 1D) and Cellastim made using the old and new processes shown in FIG. 1E.

The proteins corresponding to these peaks represent about 5% of all of the contaminant proteins identified by Peptide Mass Fingerprinting analysis of the main albumin peak in Cellastim produced using the process described in Example 2, as discussed further below.

All albumin products tested also contained a peak at around 130 kDa that most likely represents albumin dimers, it is noticeable that the Cellastim dimer peak is significantly smaller than the plasma derived albumin. The creation of aggregated albumin is an indicator of protein degradation which is used as one marker for degradation or loss of stability industry wide. It is likely that the Hsps present in Cellastim promote the disaggregation of the albumin, therefore reducing the number of dimers, since it is a commonly known function of Hsp 70 and other Hsp proteins.

### II. Analysis by SDS PAGE of Cellastim Batch P0171 (New Process) Compared to Albumin Produced by Millipore/Novozymes (Cat No. 9301-01).

Results:

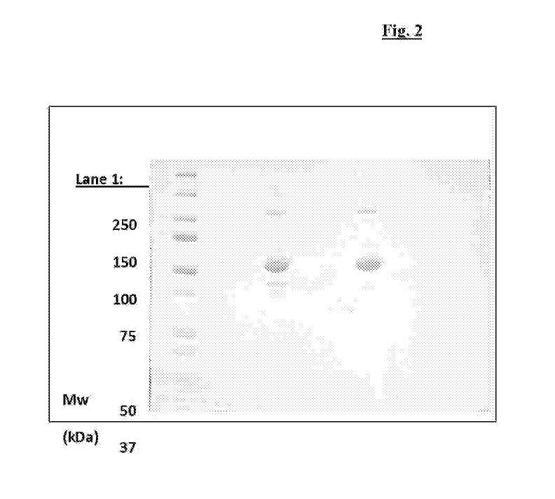

SDS-PAGE analysis (FIGS. 2A & B) shows that in terms of overall purity the products are generally similar. FIG. 2A shows a comparison of Cellastim P0171 and Cellprime albumin (Millipore/Novozymes). Lane 1 is the molecular weight marker. Lane 4 is the Cellastim albumin (10 μg) and Lane 7 is the Cellprime albumin (10 μg). FIG. 2B shows a comparison by SDS PAGE analysis of three Cellastim lots from the previous process (B000) (Lane 2, 3, and 4), and the new Cellastim Process (B0000C) (Lane 6, 7, and 8). The six samples were loaded at 20 μg per lane.

Visual inspection of the gel shows that the new process which meets more rigorous specifications is more consistent among the 3 lots tested. (FIG. 2B, lane 2, 3, 4 vs. lane 6, 7, 8). The banding pattern is significantly different among the three samples from the previous process as compared to the new process. Importantly, the new process samples have significantly less aggregates at around 250 KDa than the old process samples have. (Average greater than 2% for the old process, and average less than 1% for the new process). The identity of the protein contaminates was that are enriched in Cellastim produced using the new process is discussed further below.

### III. Analysis of Endotoxin, Detergent and Growth Promoting Abilities of Old and New Batches of Cellastim.

A comparison of the performance of the two different processes for preparing several different lots of albumin (Tables E1 and E2) demonstrates that the old process produced recombinant albumin that contained significantly more endotoxin, and detergent compared to the new process described in Example 2, and resulted in a product that significantly enhanced cell viability.

TABLE E1
Cellastim - Old Process B000

| | | | EU/mg | | | | | |
|---|---|---|---|---|---|---|---|---|
| Production | Batch | Grams | Dry | Detergent | Viability (number of viable cells/ml/10⁵) | | | |
| Date | Number | product | Material | (ppm) | 1 mg/ml | 2 mg/ml | 5 mg/ml | 10/mg ml |
| Feb. 27, 2008 | B202 | 70.6 | 25.3 | 3313 | 15 | 7.9 | 4.1 | 0.9 |
| Feb. 27, 2008 | B203 | 58.8 | 23.1 | 946 | 15.8 | 12.4 | 6.5 | 4.1 |
| Feb. 28, 2008 | B204 | 59.9 | 29.2 | 1371 | 16.3 | 11.1 | 5.2 | 2.9 |
| Feb. 28, 2008 | B205 | 63 | 64.3 | 1250 | 14 | 11.6 | 6.9 | 3.8 |
| Mar. 3, 2008 | B206 | 41.1 | 35.1 | 11602 | 14.9 | 2.3 | 0.0 | 0.0 |
| Mar. 3, 2008 | B207 | 94.2 | 28.7 | 750 | 16.5 | 12.7 | 7.6 | 4 |
| Mar. 4, 2008 | B208 | 24.5 | >80 | 1250 | 15.3 | 11.4 | 7.1 | 2.7 |
| Mar. 4, 2008 | B209 | 66.1 | 91.8 | 595 | 16.7 | 15.2 | 6.9 | 4.1 |
| Mar. 4, 2008 | B210 | 87.3 | 67.8 | 77 | 17.5 | 19.1 | 15.4 | 8.5 |
| Mar. 11, 2008 | B217 | 62.09 | 4.4 | 430.5 | 16.7 | 18.7 | 13.2 | 6.6 |

| | | | | | | |
|---|---|---|---|---|---|---|
| Averages | 44.97 | 2158.5 | 15.87 | 12.24 | 7.29 | 3.76 |

TABLE E2
Cellastim - New Process B0000C

| | | | EU/mg | | | | | |
|---|---|---|---|---|---|---|---|---|
| Production | Batch | Grams | Dry | Detergent | Viability (number of viable cells/ml/10⁵) | | | |
| Date | Number | product | Material | (ppm) | 0.5 mg/ml | 2 mg/ml | 5 mg/ml | 10/mg ml |
| Feb. 2, 2009 | B0032C | 209.4 | 0.35 | 170 | 13.1 | 15.7 | 13.4 | 11.2 |
| Feb. 2, 2009 | B0033C | 271.4 | 0.26 | not det. | 18.4 | 17.4 | 15.1 | 14.3 |
| Feb. 10, 2009 | B0041C | 247.1 | 0.18 | 93 | 15.4 | 17.2 | 14.1 | 12.1 |
| Apr. 27, 2009 | B0118C | 617.4 | 0.11 | not det | 15.7 | 15.4 | 19.2 | 16.8 |
| May 14, 2009 | B0138C | 610.6 | 0.48 | 102 | 12.5 | 15.8 | 15.1 | 14.6 |
| Jun. 9, 2009 | B0158C | 598.0 | 0.13 | 141 | 13.7 | 17.1 | 13.2 | 11 |
| Jun. 15, 2009 | B0162C | 618.6 | 0.20 | not det. | 15.5 | 17.5 | 17.2 | 14.7 |
| Jul. 28, 2009 | B0196C | 507.0 | 0.36 | not det. | 17.4 | 17.3 | 18.8 | 14.8 |
| Aug. 26, 2009 | B0219C | 851.4 | 0.86 | not det. | 17.9 | 16.3 | 16.6 | 13.7 |
| Aug. 31, 2009 | B0220C | 897.8 | 0.93 | 77 | 13.5 | 16 | 15.5 | 15.1 |
| Sep. 9, 2009 | B0227C | 929.9 | 0.13 | 270 | 11.1 | 14.4 | 12.5 | 11.4 |

| | | | | | | |
|---|---|---|---|---|---|---|
| Averages | 0.36 | 142.2 | 14.92 | 16.37 | 15.52 | 13.6 |

Discussion:

Re-engineering the old process to create the new process described in Example 2 resulted in significant changes in both overall product purity, and performance, as described more fully below.

The changes made it possible to make products that were lower in detergent, lower in endotoxin, and increased purity. Specifically, the new process routinely produced recombinant albumin with an overall purity of greater than about 95%. By comparison the old method routinely produced albumin with a maximum purity of about 90%. Surprisingly, despite the increased product purity, these changes in processing also resulted in enhanced co-purification of heat shock proteins, (see below) with the recombinant albumin. Without being bound by any particular theory of operation, it is believed that the combination of high albumin purity, relative lack of endotoxin and/or detergent, and co-purification of heat shock proteins results in a product that significantly out performs previous methods for preparing albumin.

Specifically Tables E1 and E2 demonstrate that the new process for producing Cellastim results in a product that, for example at 5 mg/ml, results in an average batch to batch 100 percent improvement in cell viability (at 5 mg/ml), and also results in a product with an average 100-fold less endotoxin, and 100 fold less detergent than the old process.

### Example 4

### Analysis of the Effects on Cell Growth and Viability

To compare the cell growth promoting abilities of the supplements of the invention, to other commercially available albumin products, the different sources of albumin they were compared side by side in a cell growth and viability assay. The three products tested were (Cellastim, Lot # P0153) Cellprime albumin (Millipore/Novozymes Cat No. #9301-01), and plasma derived albumin (Seracare Cat No. #HS -400-60).

Methods:

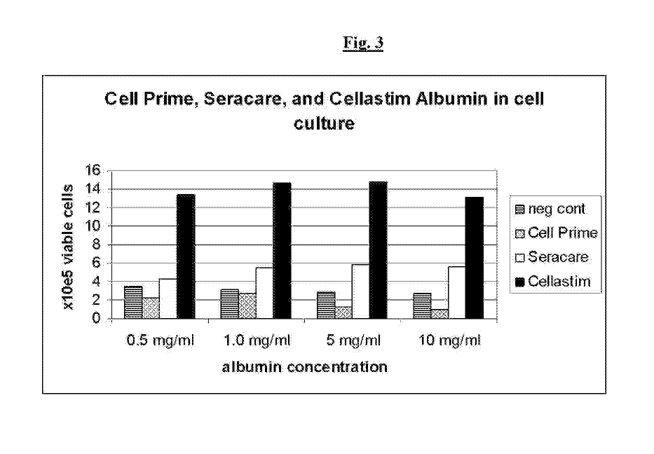

Specially conditioned Hybridoma cells AE1 were seeded in DF12/ITSE at a density of 0.5×10⁵ cells per ml of media after washing twice with same media to remove residual media. The media and cells were then left untreated (negative control), treated with Seracare albumin, treated with Cellprime albumin, and treated with Cellastim at the concentrations shown in the figure legend. The cells were grown under standard culture conditions (5% CO₂ and 37° C.) for approximately 70 hours after which the viability for the cultures was measured. The experiments were conducted in duplicate. Results are shown in FIG. 3.

Results:

Novazyme's Cellprime caused a loss in viability (cross-hatch bars). Seracare albumin (white bars) caused a measureable increase in viability but not as large an increase as is seen with the supplement of the invention comprising recombinant albumin with rice hsps (black bars). Under these conditions Cellastim was approximately 5 times as active in promoting cell viability compared to any of the other albumin products, at any concentration tested. The negative control is represented by the striped bars.

Discussion:

Given the possibility that the other commercially available albumin products may have similar overall purity, endotoxin and detergent levels to Cellastim, the dramatically superior performance of the recombinant albumin of the invention compared to other commercially available albumins suggests that the previously un-identified protein contaminants identified in Cellastim compared to the serum derived albumin (Example 2) could be having a positive impact on cell viability. To identify and then characterize the impact of these proteins on the properties of Cellastim, a sample of the recombinant albumin was subjected to peptide mass finger printing, as described below.

### Example 5

### Peptide Mass Finger Printing of Recombinant Human Serum Albumin

Methods:

Samples of albumin were analyzed to determine significant protein contaminants using a NanoLCMS/MS peptide sequencing system (ProtTech, Inc.), and proprietary software to identify the proteins based on the molecular weight of the peptide fragments. In brief, samples of albumin were analyzed by SDS-PAGE, and each major band gel band was destained, cleaned, and digested in-gel with sequencing grade modified trypsin. The resulting peptide mixture was analyzed by a LC-MS/MS system, in which a high pressure liquid chromatography (HPLC) with a 75 micrometer inner diameter reverse phase C18 column was used in-line coupled with an ion trap mass spectrometer. The mass spectrometric data acquired was used to search the most recent non-redundant protein database with ProtTech's proprietary software suite. The output from the database search was manually validated before reporting.

Results:

Upon testing of three representative lots of recombinant albumin, three Hsp70 proteins were identified by Peptide Mass Fingerprinting (Table E3). The three specific sequences identified: ABF95267, ABA97211, and BAD 07938 were compared to the non redundant database to identify highly related and homologous proteins. The results of the top hits from each of these comparisons is shown in Tables E4, E5 and E6

TABLE E3
Peptides identified from Cellastim by
mass finger printing

| Sequence | Peptide |
|---|---|
| ABF95267 | ATAGDTHLGGEDFDNRVVPGPADKSPMIVVTYKGEEK |
| | NAVITVPAYFN DSQRIINEPTAAAIAYGLDKK |
| | (SEQ. ID. NO. 9) |
| AAB63469 | NQAAVNPER NGHVEIIANDQGNRIVNKDGKPYIQVK |
| BAD07938 | IINEPTAAAIAYGLDKK KLGTVIGIDLGTTYSCVGVYK |
| BAD07713 | VEIESLFDGTDSFSEPLTR |
| | (SEQ. ID. NO. 10) |
| ABA97211 | NQADSVVYQTEKKQDITITGASTLPKDEVERDVVLLDVT |
| | PLSLSLGLETLGGVMTK |
| | (SEQ. ID. NO. 11) |

Results of sequence comparisons to ABF95267 sequences in the non redundant database of protein sequences in Genbank® (Nucleic Acids Research, 2008 January; 36 (Database issue):D25-30) are shown in Table E4 below.

TABLE E4
Sequences producing significant alignments with ABF95267:

| Gene Refs | Gene description | (Bits) | Value |
|---|---|---|---|
| ref\|NP_001140835.1\| | hypothetical protein LOC100272911 [Zea ma . . . | 79.7 | 8e-14 |
| ref\|XP_002465468.1\| | hypothetical protein | 79.7 | 9e-14 |
| | SORBIDRAFT_01g039390 . . . | | |
| ref\|NP_001049719.1\| | Os03g0277300 [Oryza sativa (japonica cult . . . | 79.7 | 9e-14 |
| gb\|ACJ54890.1\| | heat shock protein 70 [Oryza sativa Japonica | 79.7 | 9e-14 |
| | G . . . | | |
| sp\|P09189.1\| | HSP7C_PETHY RecName: Full = Heat shock | 77.0 | 5e-13 |
| | cognate 70 k . . . | | |
| emb\|CAA31663.1\| | hsp70 (AA 6-651) [Petunia × hybrida] | 77.0 | 5e-13 |
| ref\|XP_002312089.1\| | predicted protein [Populus trichocarpa] > . . . | 77.0 | 5e-13 |
| sp\|P24629.1\| | HSP71_SOLLC RecName: Full = Heat shock | 77.0 | 5e-13 |
| | cognate 70 k . . . | | |
| gb\|AAB99745.1\| | HSP70 [Triticum aestivum] | 76.6 | 6e-13 |
| gb\|AAB42159.1\| | Hsc70 [Lycopersicon esculentum] | 76.6 | 7e-13 |
| gb\|ACD45076.1\| | heat-shock protein 70 [Dactylis glomerata] | 76.3 | 8e-13 |
| ref\|XP_002512741.1\| | heat shock protein, putative [Ricinus com . . . | 75.9 | 1e-12 |
| ref\|XP_002512742.1\| | heat shock protein, putative [Ricinus com . . . | 75.9 | 1e-12 |
| gb\|AAA82975.1\| | PsHSP71.2 > emb\|CAA67867.1\| heat shock | 75.9 | 1e-12 |
| | protein . . . | | |
| gb\|AAS09825.1\| | heat shock cognate protein 70 [Thellungiella | 75.9 | 1e-12 |
| | h . . . | | |
| emb\|CAA44820.1\| | heat shock protein 70 [Nicotiana tabacum] | 75.5 | 2e-12 |
| ref\|NP_001055754.1\| | Os05g0460000 [Oryza sativa (japonica cult . . . | 75.5 | 2e-12 |
| ref\|NP_001051724.1\| | Os03g0821100 [Oryza sativa (japonica cult . . . | 75.1 | 2e-12 |
| ref\|XP_002456611.1\| | hypothetical protein | 75.1 | 2e-12 |
| | SORBIDRAFT_03g039360 . . . | | |
| ref\|NP_001044757.1\| | Os01g0840100 [Oryza sativa (japonica cult . . . | 75.1 | 2e-12 |
| gb\|ACR35910.1\| | unknown [Zea mays] | 75.1 | 2e-12 |
| ref\|XP_002284017.1\| | PREDICTED: similar to HSC70-1 (heat | 75.1 | 2e-12 |
| | shock . . . | | |
| ref\|XP_002532297.1\| | heat shock protein, putative [Ricinus com . . . | 75.1 | 2e-12 |
| ref\|XP_002284008.1\| | PREDICTED: similar to HSC70-1 (heat | 75.1 | 2e-12 |
| | shock . . . | | |
| ref\|XP_002283532.1\| | PREDICTED: similar to HSC70-1 (heat | 74.7 | 3e-12 |
| | shock . . . | | |
| ref\|XP 002332067.1\| | predicted protein [Populus trichocarpa | 74.7 | 3e-12 |
| gb\|AAF34134.1\| | high molecular weight heat shock protein | 74.7 | 3e-12 |
| | [Malu . . . | | |
| gb\|EEC76425.1\| | hypothetical protein OsI_14101 [Oryza sativa | 74.7 | 3e-12 |
| | I . . . | | |
| ref\|XP_002316294.1\| | predicted protein [Populus trichocarpa] > . . . | 74.7 | 3e-12 |
| ref\|XP_002283516.1\| | PREDICTED: similar to HSC70-1 (heat | 74.3 | 3e-12 |
| | shock . . . | | |
| ref\|XP_002441219.1\| | hypothetical protein | 74.3 | 4e-12 |
| | SORBIDRAFT_09g022580 . . . | | |

Results of sequence comparisons of ABB63469 to sequences in the non redundant database of protein sequences in GenBank® are shown in Table E5 below.

TABLE E5
Sequences producing significant alignments with AAB63469:

| Gene Refs | Gene description | (Bits) | Value |
|---|---|---|---|
| emb\|CAP31983.1\| | C. briggsae CBR-HSP-4 protein | 50.1 | 7e-05 |
| | [Caenorhabditis . . . | | |
| dbj\|BAG60366.1\| | unnamed protein product [Homo sapiens] | 49.7 | 8e-05 |
| ref\|YP_002421952.1\| | chaperone protein DnaK [Methylobacterium . . . | 49.7 | 9e-05 |
| ref\|YP_001640420.1\| | chaperone protein DnaK [Methylobacterium . . . | 49.7 | 9e-05 |
| ref\|NP_001105893.1\| | Binding protein homolog1 precursor [Zea m . . . | 49.3 | 1e-04 |
| gb\|AAA62325.1\| | HSP70 | 49.3 | 1e-04 |
| ref\|YP_001756576.1\| | chaperone protein DnaK [Methylobacterium . . . | 49.3 | 1e-04 |
| gb\|AAB63469.1\| | endosperm lumenal binding protein [Oryza | 49.3 | 1e-04 |
| | sativa] | | |
| ref\|YP_001925829.1\| | chaperone protein DnaK [Methylobacterium . . . | 49.3 | 1e-04 |
| ref\|NP_001105894.1\| | Binding protein homolog2 precursor [Zea m . . . | 49.3 | 1e-04 |
| gb\|ACF86491.1\| | unknown [Zea mays] | 49.3 | 1e-04 |
| ref\|NP_001045675.1\| | Os02g0115900 [Oryza sativa (japonica cult . . . | 49.3 | 1e-04 |
| ref\|ZP_02191025.1\| | Molecular chaperone [alpha proteobacterium . . . | 49.3 | 1e-04 |
| ref\|XP_001701685.1\| | binding protein 1 [Chlamydomonas reinhard . . . | 48.5 | 2e-04 |
| ref\|XP_001701884.1\| | binding protein 2 [Chlamydomonas reinhard . . . | 48.5 | 2e-04 |
| emb\|CAC37635.1\| | luminal binding protein, BiP [Scherffelia dubia] | 48.1 | 3e-04 |
| gb\|AAM93256.1\| | heat shock protein 70-C [Heterodera glycines] . . . | 48.1 | 3e-04 |

Results of sequence comparisons of sequence ABA97211 to sequences in the non redundant database of protein sequences in GenBank® are shown in Table E6 below.

TABLE E6
Sequences producing significant alignments with ABA97211

| Gene Refs | Gene description | (Bits) | Value |
|---|---|---|---|
| gb\|AAK13022.1\| | heat shock protein 70 [Fibrobacter | 44.3 | 0.004 |
| | succinogene . . . | | |
| ref\|XP_002442079.1\| | hypothetical protein | 44.3 | 0.004 |
| | SORBIDRAFT_08g009580 . . . | | |
| gb\|EEC69073.1\| | hypothetical protein OsI_37938 [Oryza sativa | 44.3 | 0.004 |
| | I . . . | | |
| ref\|XP_001752769.1\| | predicted protein [Physcomitrella patens . . . | 44.3 | 0.004 |
| ref\|NP_001066486.1\| | Os12g0244100 [Oryza sativa (japonica cult . . . | 44.3 | 0.004 |
| gb\|ACT65562.1\| | 70 kDa heat shock protein [Triticum aestivum] | 43.9 | 0.005 |
| ref\|NP_001152528.1\| | stromal 70 kDa heat shock-related protein . . . | 43.9 | 0.005 |
| gb\|ACN31310.1\| | unknown [Zea mays] | 43.9 | 0.005 |
| ref\|XP_001772650.1\| | predicted protein [Physcomitrella patens . . . | 43.9 | 0.005 |
| ref\|NP_001146752.1\| | hypothetical protein LOC100280354 [Zea ma... | 43.9 | 0.005 |
| gb\|ABP65327.1\| | chloroplast heat shock protein 70 | 43.9 | 0.005 |
| | [Pennisetum . . . | | |
| gb\|AAO72585.1\| | heat shock-related protein [Oryza sativa (japo . . . | 43.5 | 0.007 |
| ref\|YP_001740846.1\| | Chaperone protein dnaK (Heat shock protei . . . | 43.5 | 0.008 |
| ref\|ZP_03728467.1\| | chaperone protein DnaK [Dethiobacter alkal . . . | 43.1 | 0.009 |

Discussion:

Peptide Mass Fingerprinting identified 3 rice heat shock protein super family members that co-purify with albumin, 2 Rice HSP70 genes, (gblACJ54890.11), EEC69073, and AAB63469—a BiP homolog from rice endosperm tissue (endosperm lumenal binding protein). The complete amino acid sequences coded by these genes are listed below:

Gene gblACJ54890.1l heat shock protein 70 [Oryza sativa Japonica Group] HSP70 was found to occur in recombinant albumin in Cellastim at approximately 0.07% wt/wt. Its complete amino acid coding sequence is provided below:

```
(SEQ. ID. NO. 12)
    1 magnkgegpa igidlgttys cvgvwqhdry eiiandqgnr ttpsyvaftd terligdaak
  61 nqvamnptnt vfdakrligr rfsdpsvqad mkmwpfkvvp gpadkpmivv tykgeekkfs
121 aeeissmvlt kmkeiaeafl sttiknavit vpayfndsqr qatkdagvis glnvmriine
181 ptaaaiaygl dkkaastgek nvlifdlggg tfdvsiltie egifevkata gdthlggedf
241 dnrmvnhfvq efkrkhkkdi tgnpralrrl rtacerakrt lsstaqttie ieslyegidf
301 yatitrarfe elnmdlfrrc mepvekclrd akmdkaqihd vvlvggstri pkvqqllqdf
361 fngkelcksi npdeavayga avqaailsge gnqrvqdlll ldvtplslgl etaggvmtvl
421 iprnttiptk keqvfstysd nqpgvliqvy egertrtkdn nllgkfeltg ippaprgvpq
481 invtfdidan gilnvsaedk ttgkknkiti tndkgrlske eiermvqeae kykaedeqvr
541 hkvearnale nyaynmrntv rdekiasklp addkkkieda iedaikwldg nqlaeadefe
601 dkmkeleslc npiiskmyqg gaggpagmde dapngsagtg ggsgagpkie evd
```

AAB63469 BiP homolog from rice endosperm tissue (endosperm lumenal binding protein [Oryza sativa]) BiP was found to occur in recombinant albumin in Cellastim at about 0.09% wt/wt. Its complete amino acid coding sequence is provided below:

```
(SEQ. ID. NO. 13)
    1 mdrvrgsafl lgvllagslf afsvakeetk klgtvigidl gttyscvgvy knghveiian
  61 dqgnritpsw vaftdserli geaaknqaav npertifdvk rdigrkfeek evqrdmklvp
121 ykivnkigkp yiqvkikdge nkvfspeevs amilgkmket aeaylgkkin davvtvpayf
181 ndaqrqatkd agviaglnva riineptaaa iaygldkkgg eknilvfdlg ggtfdvsilt
241 idngvfevla tngdthlgge dfdqrimeyf iklikkkysk diskdnralg klrreaerak
301 ralsnqhqvr veieslfdgt dfsepltrar feelnndlfr ktmgpvkkam ddagleksqi
361 heivlvggst ripkvqqllr dyfegkepnk gvnpdeavay gaavqgsils geggdetkdi
421 llldvapltl gietvggvmt kliprntvip tkksqvftty qdqqttvsiq vfegersmtk
481 dcrllgkfdl sgipaaprgt pqievtfevd angilnvkae dkgtgkseki titnekgrls
541 qeeidrmvre aeefaeedkk vkeridarnq letyvynmkn tvgdkdklad kleseekekv
601 eealkealew ldenqtaeke eyeeklkeve avcnpiisav yqrtggapgg rrrgrlddeh
661 del
```

EEC69073/OsI_{—}37938 [Oryza sativa Indica Group] The stromal HSP70 was found to occur in recombinant albumin in Cellastim at about 0.06% wt/wt. Its complete amino acid coding sequence is provided below:

```
(SEQ. ID. NO. 14)
    1 masftsqlga macgaapsts plaarrsgql fvgrkpaaas vqmrvpragr argvamrvac
  61 ekvvgidlgt tnsavaameg gkptvitnae gqrttpsvva ytkggerlvg qiakrqavvn
121 pentffsvkr figrkmaevd deakqvsyhv vrddngnvkl dcpaigkqfa aeeisaqvlr
181 klvddaskfl ndkitkavvt vpayfndsqr tatkdagria glevlriine ptaaslaygf
241 ekknnetilv fdlgggtfdv svlevgdgvf evlstsgdth lggddfdkfy fcwvfyfgam
301 thetpkvvdw lasnfkkdeg idllkdkqal qrlteaaeka kmelstlsqt nislpfitat
361 adgpkhiett lsrakfeelc sdlidrlktp vtnalrdakl svdnldevil vggstripsv
421 qelvkkitgk dpnvtvnpde vvslgaavqg gvlagdvkdv vlldvtplsl gletlggvmt
481 kiiprnttlp tsksevfsta adgqtsvein vlqgerefvr dnkslgsfrl dgippaprgv
541 pqievkfdid angilsvaai dkgtgkkqdi titgastlpk devermveea dkfaqedkek
601 rdaidtknqa dsvvyqtekq lkelgdkvpa pvkekvdakl nelkeaiagg stqsmkdama
661 alneevmqig qamynqqpna gaagptpgad agptssggkg pndgdvidad ftdsn
```

Because these proteins only occur at low levels in the new batches of Cellastim relative to albumin, a pre-requisite for confirming that these contaminants are actually responsible for the superior growth promoting effects of the new batches of Cellastim is to determine whether the addition back of these components to albumin restores or enhances the growth promoting activities of the albumin at levels which are comparable to those actually identified for each component in Cellastim.

### Example 6

### Separation of Heat Shock Proteins from Recombinant Albumin by Affinity Chromatography

Methods:

Cellastim produced using the new process [Lots P0153, P0156, and or P0171] powder was mixed with purified water at approximately 20 g/L. The resulting solution was diafiltered against 50 mM Tris/Cl, pH 7.0 with at least 5 equal volumes of buffer. The resulting solution was passed over an ATP agarose column and the resulting flow through was labeled as fraction A. The column was washed with 5 column volumes of the equilibration buffer and the material bound to the ATP-agarose was eluted with 50 mM Tris/Cl, 1M KCl, pH7.0. The eluted material was labeled as fraction B. The wash was kept as fraction C. Fraction A was directly concentrated to 100 g/L and diafiltered with d-PBS. Fraction B was concentrated significantly, up to 20 fold or 100 fold in 50 mM Tris/Cl for further analysis. The wash fraction C was kept for further reference. For Western blotting, 10 μg of each protein fraction (by A280, where the e.c. (extinction coefficient) of albumin is 0.53 cm²/mg and e.c. of Hsp70 is 0.41 cm²/mg) were loaded on a 4-20% SDS PAGE gel in 2×SDS loading buffer. The samples were heated to 80° C. for approximately 5 minutes before loading. The separation was done at 200V (constant voltage) and ran for approximately 90 minutes. The resulting gel was rinsed in water for 30 minutes to 2 hours and then the proteins were transferred to a Nitrocellulose membrane at 30 mA (constant current) for 2 hours. The resulting blot contained the molecular weight marker proteins as a transfer control and was then blocked in 5% (w/v) milk powder in water. The primary monoclonal antibody (a mouse anti-bovine Hsp70 (Sigma/Aldrich #H5147)) was added in 5% milk solution to the blot (1:2500) and the blot was incubated on a rocker with gentle rocking overnight at 4° C. The blot was then washed 4 times for 10 minutes each in TDN and the secondary antibody (Pierce anti-mouse HRP conjugated) in 5% milk solution which was added at a dilution of 1:2500. After incubation at 4° C. for 2 to 3 hours, the blot was washed 4 times with TDN for 10 minutes each. The resulting blot was then incubated with pico (Pierce) chemiluminescent substrate for 5 minutes. Kodak photographic film was exposed to the blot in a dark room and the subsequent film was developed, rinsed, fixed, rinsed, and dried. To determine accurate transfer of the molecular weight marker position onto the film, a light emitting label was used.

Results:

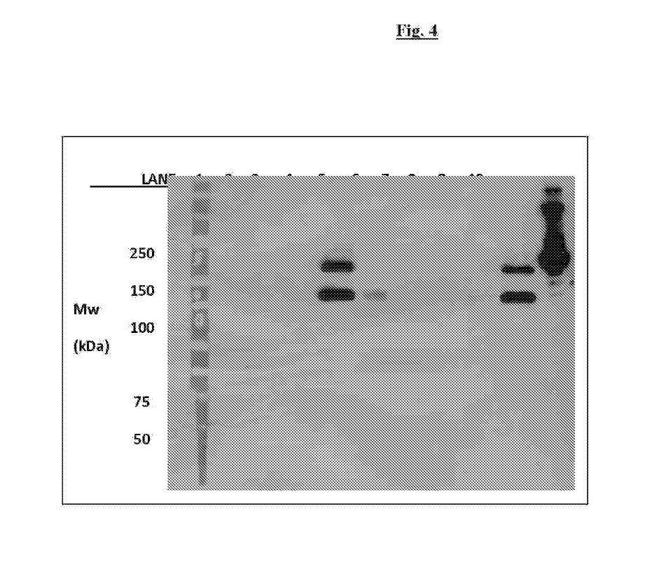

The results are shown in FIG. 4. The Western blot pictured shows that the separation scheme produces two populations of proteins in the A (flow through) and B (ATP binding) fractions. The starting material, (lane 2) the fraction A flow through, (lane 3) fraction C wash, (lane 4) and fraction B (lane 5) were tested for the ability to react to the monoclonal antibody. In addition, a commercially available Hsp70 protein that serves as a positive control was loaded in the last lane (lane 10). As shown in the blot in FIG. 4, the flow through fraction A (lane 3) does not contain significant amounts of Hsp70. The eluted and concentrated fraction B (lane 4) is highly reactive to the antibody as shown in the blot and indicates at least two distinct bands centered around the 75 kDa molecular weight marker. The wash fraction C (lane 5), indicates the presence of two bands that run at slightly below 75 kDa. In a separate independent experiment, the flow through fraction A (lane 7) again is not reactive to the antibody, and the wash fraction C (lane 8) is also not reactive to the antibody, but the fraction enriched in ATP binding proteins (Fraction B) shown in lane 9 gives the same banding pattern as was seen from the first separation.

Discussion:

A separation protocol was developed to separate HSP70 proteins based on their ability to bind to ATP agarose affinity resin, and, was tested for its effectiveness. The procedure involved only minimal sample manipulation, using only ATP agarose, and ultrafiltration to concentrate and conduct buffer changes, and an anti-hsp70 antibody to detect the presence of hsps. The results of the procedure (FIG. 4) clearly demonstrates that while in 10 μg of starting material, 10 μg of flow through, or 10 μg of wash fraction there is insufficient hsp70 to be detected by the ant-Hsp antibody. By contrast, in the fraction eluted from the ATP agarose column, contains at least two proteins that clearly are recognized by the anti-Hsp70 antibody. The results therefore show that the separation scheme was successful and predictable on two independent chromatography runs and diafiltrations. Furthermore, the data substantiates the identification of heat shock proteins made by Peptide Mass Finger printing and demonstrates that these proteins are functional and can be readily isolated and enriched by simple ATP agarose chromatography followed with diafiltration. It is concluded that the heat shock proteins co-purify with the recombinant albumin. Such co-purification is consistent with the hypothesis that the heat shock proteins are bound to the albumin, and that the albumin acts to stabilize the heat shock proteins in a stable conformation. Surprisingly, the recombinant albumin/heat shock protein complex retains significant ATPase activity (data not shown) consistent with the presence of function heat shock proteins. This increased activity was further confirmed as providing a growth promoting effect as described below.

### Example 7

### Impact of the Removal of Hsps from Cellastim on Cell Viability

Methods:

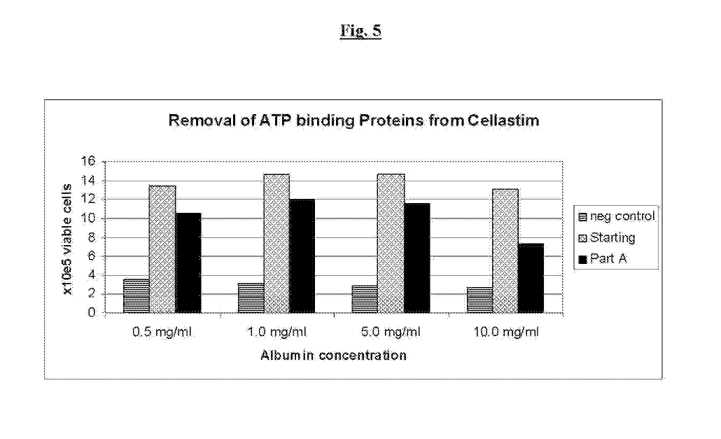

The separation scheme described in Example 6 was also used to produce fraction A suitable for Cell culture testing (FIG. 5). The method involves minimal manipulation of fraction A, as it is flowed through an ATP agarose column and then concentrated by diafiltration and buffered with PBS that is suitable for cell culture. The intent of the method is to not introduce new variables into the experiment such that a loss of viability is seen but due to some other reason or cause beyond the removal of ATP binding proteins. Fraction A was tested against the unadulterated control (starting material) for ability to promote hybridoma cell culture viability. The results of the test are shown in FIG. 5.

Results:

As shown in FIG. 5 the Cellastim starting material (cross hatched bars), and Part A (solid bars) were tested at the same concentration and compared to the negative control (striped bars). A statistically significant decrease is observable at all four concentrations tested. The result indicates that there was a significant loss in the performance of Cellastim after ATP agarose treatment. The treatment resulted in a 28.0, 21.7, 26.7, and 79.5% loss as compared to Cellastim before removal of ATP binding proteins. In this experiment, care was taken in the design and handling of the samples to ensure that any inadvertent losses in performance due to sample handling, or the accidental introduction of new contaminants were minimized.

Discussion:

The cell culture results (FIG. 5) demonstrate that it is possible to reduce the performance of Cellastim by simply passing it over an ATP binding column. This data, when combined with the results shown in Example 5 demonstrates that the depletion of the hsps from albumin by the ATP agarose column directly reduces the cell growth promoting properties of the albumin. This result therefore demonstrates that the superior properties of the albumin arise, at least in part, from the contaminating heat shock proteins in the albumin.

### Example 8

### Analysis of the Effects on Cell Growth and Viability in Shaking Culture

To determine the effect of supplements of the invention on cell growth and viability when cells are grown at high density in shaking flasks and bioreactors, a series of studies were compared to directly.

Methods:

CHO K1 cells, expressing a humanized monoclonal antibody, were adapted for 6 weeks to serum-free base medium (SFM4CHO, Thermo Scientific Hyclone) containing 10 mg/L insulin) prior to study. The adapted cells were grown in shake flasks for banking. Cells were banked and stored in liquid nitrogen in a cryopreservation medium comprised of growth medium with DMSO 8% v/v.

In shake-flasks experiments, cells were seeded in the base medium or in medium containing supplements in 30 ml of medium in 125 ml/shake flasks Corning #431405 at a concentration of 3.0×10⁵ viable cells/ml. Cells were maintained at 37° C., in a humidified CO₂ incubator, at 110 RPM for the length of the run. Fed batch bioreactor experiments were conducted in 1 L or 2 L Applikon bioreactors (Applikon Biotechnology, NE) in base medium or in base medium with supplements. Cells were seeded on Day 0 at 3.0×10⁵ viable cells/ml. The bioreactor temperature, pH and dissolved oxygen (DO) was monitored and controlled by automated controllers. The reactor temperature was maintained at 37° C. by a heating blanket. The culture pH was maintained at 7.1 by the addition of CO₂ or 6% Na₂CO₃. Aeration was performed through a cylindrical sintered sparger at 10 ml/min. Dissolved oxygen was controlled at 50% of air saturation by intermittent sparging of O₂ into the medium. The agitation rate of the impeller was maintained at 180 RPM.

During the cultivation, bioreactor samples were taken periodically for off-line analysis. The viable cell density (VCD) and the cell viability were measured by membrane exclusion of a 0.4% trypan blue and cell counting with a Beckman ViCell cell counter. In some cases viable cell density and cell viability were measured by exclusion of a cell viability die, Viacount reagent (Millipore) followed by analysis on a Guava PC^{A} cell counter as directed by the manufacturer. Glucose, and lactate, concentrations were measured using standard clinical analysis using a Nova 400 Bioprofile analyzer. Specific net growth rates and specific net death rates were determined by Gaudy et al. (Guady, A F, A. Obaysahi, and E. T. Gaudy. 1971. Applied Microbiology, 22(6): p. 1041-1047). The antibody concentration was determined by anti-human IgG ELISA according to the manufacturer's directions (Bethyl Laboratories). Media supplements included recombinant human albumin, (Cellastim as described above in Example 2), or recombinant human Lactoferrin (rLF, Lacromin (L)), or a combination of both proteins. Supplements were added at cell seeding at day 0 unless otherwise indicated. Multiple experiments were conducted in both the shake-flasks and bioreactor systems under the same parameters above except where noted.

Results:

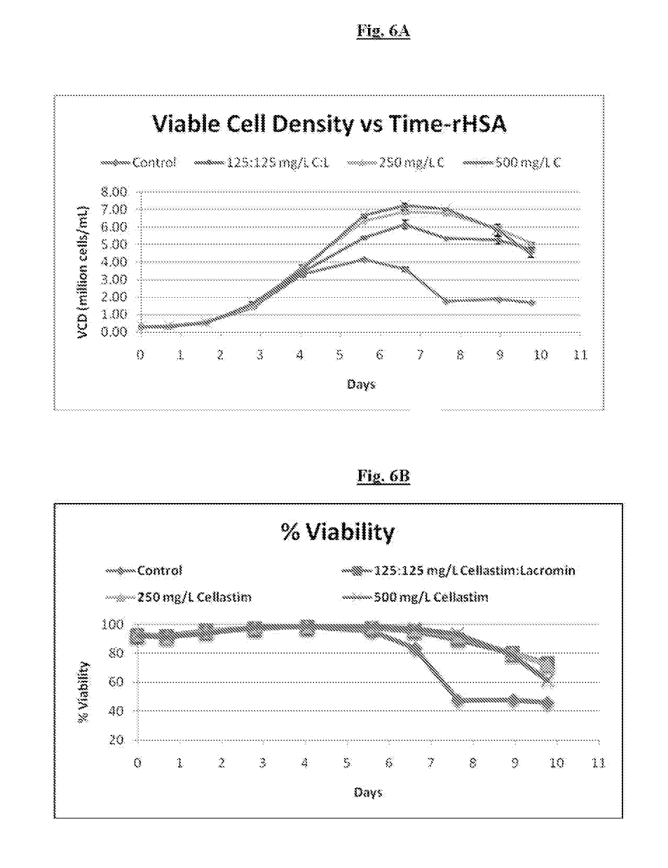

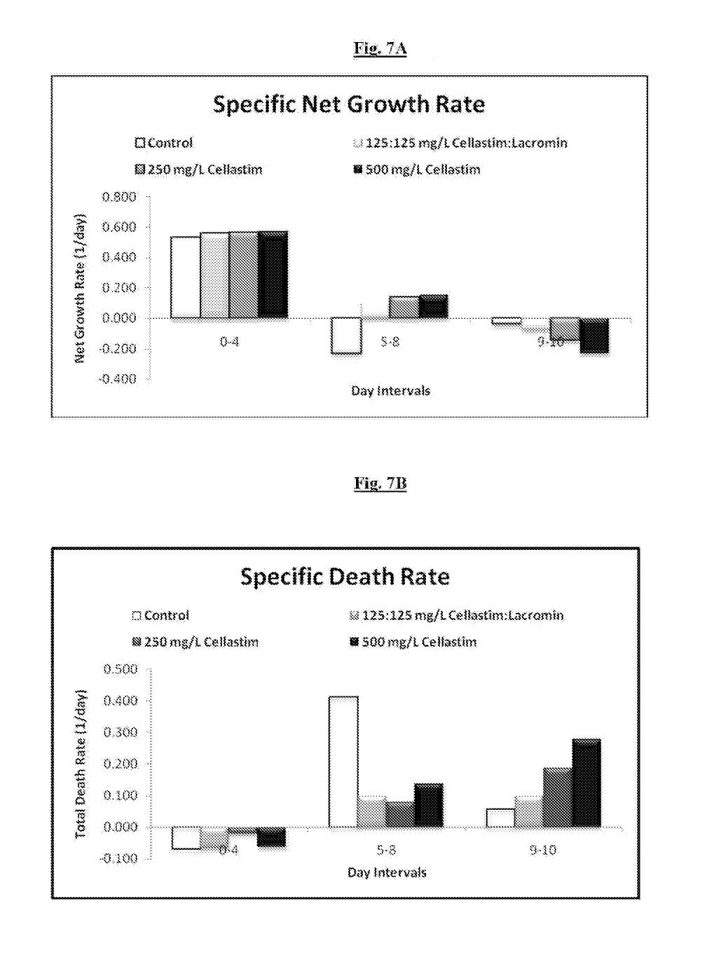

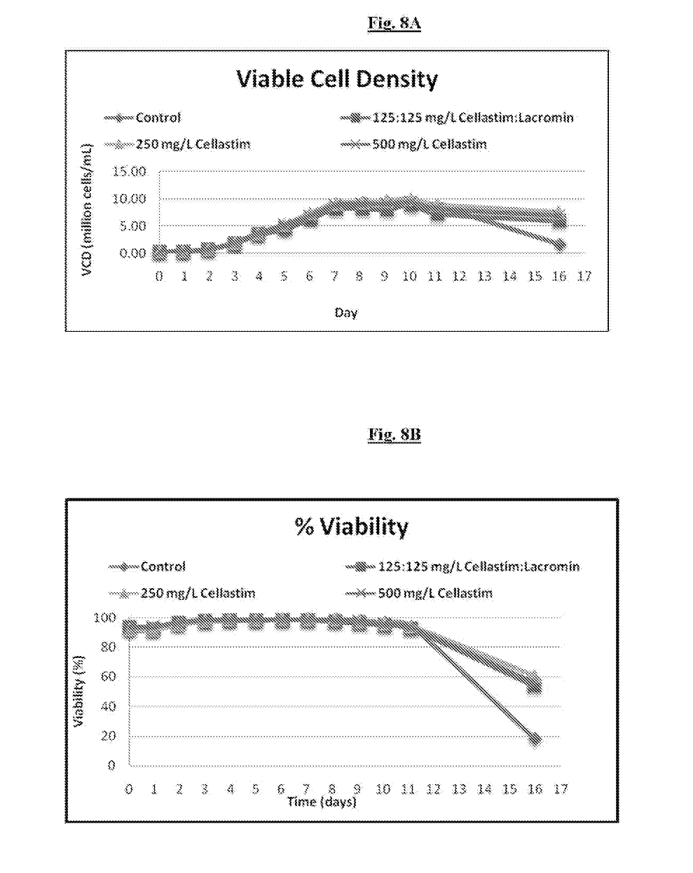

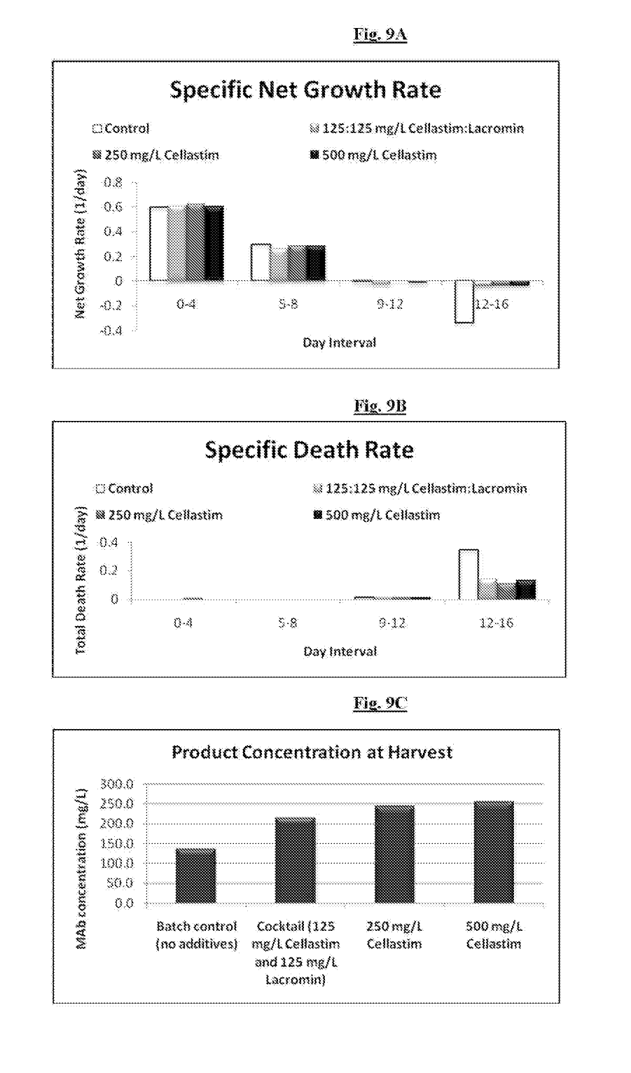

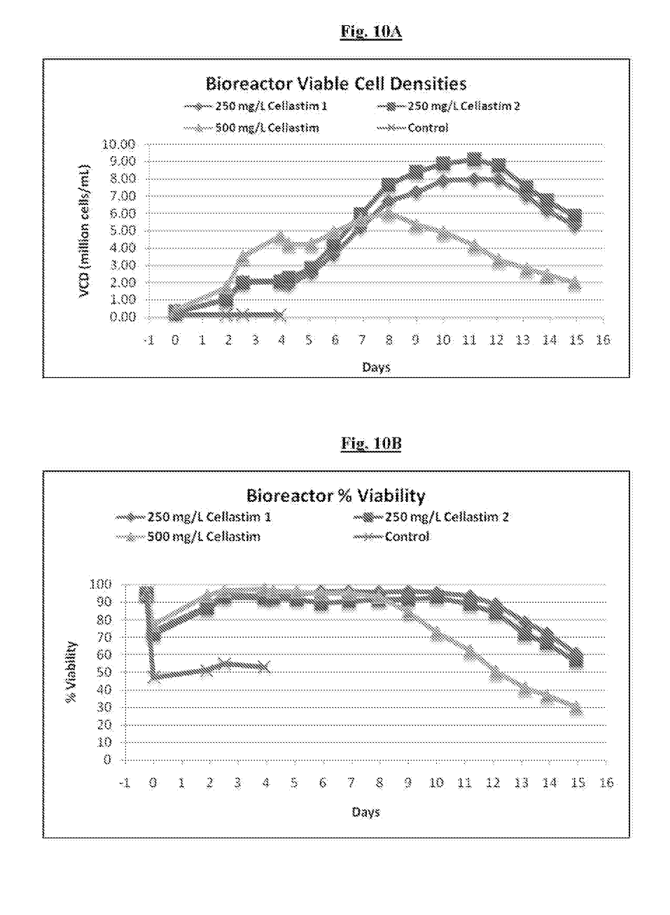

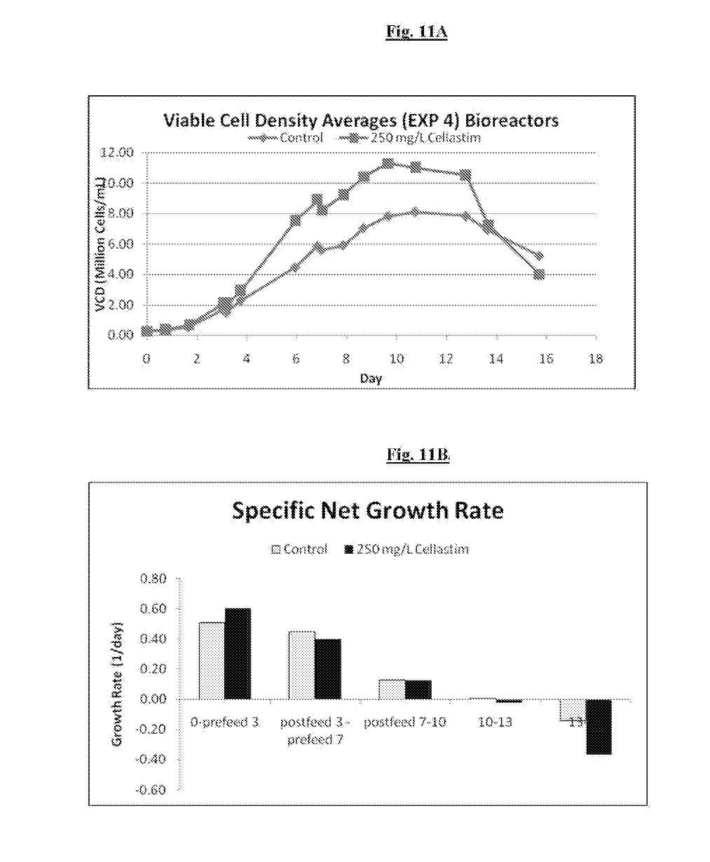

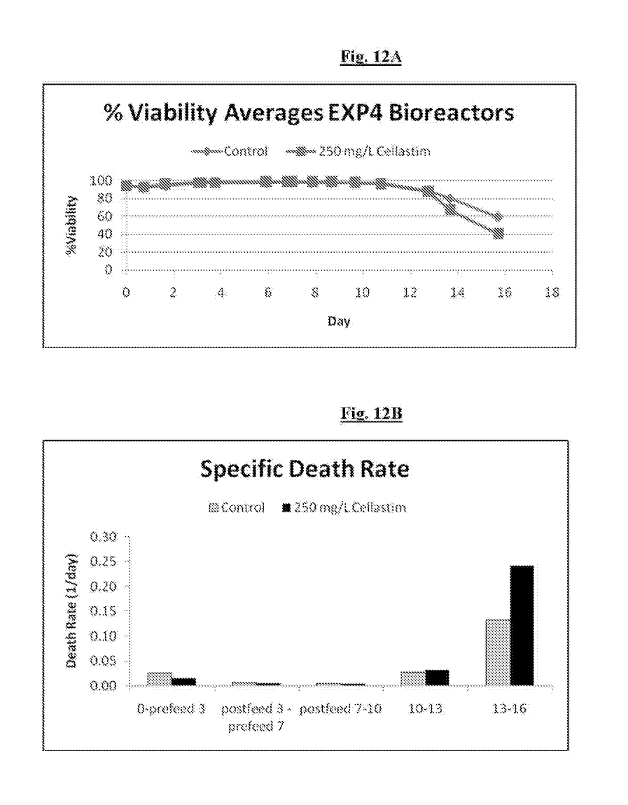

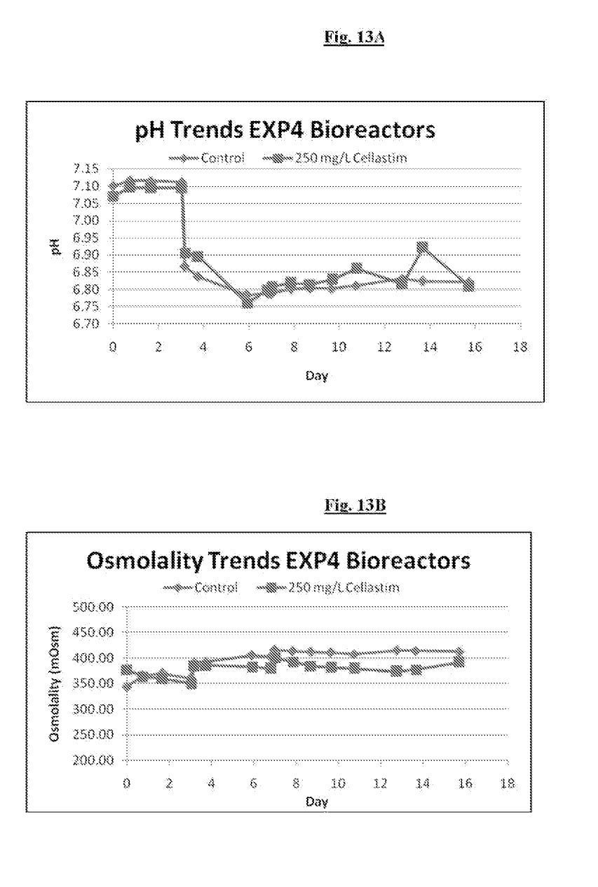

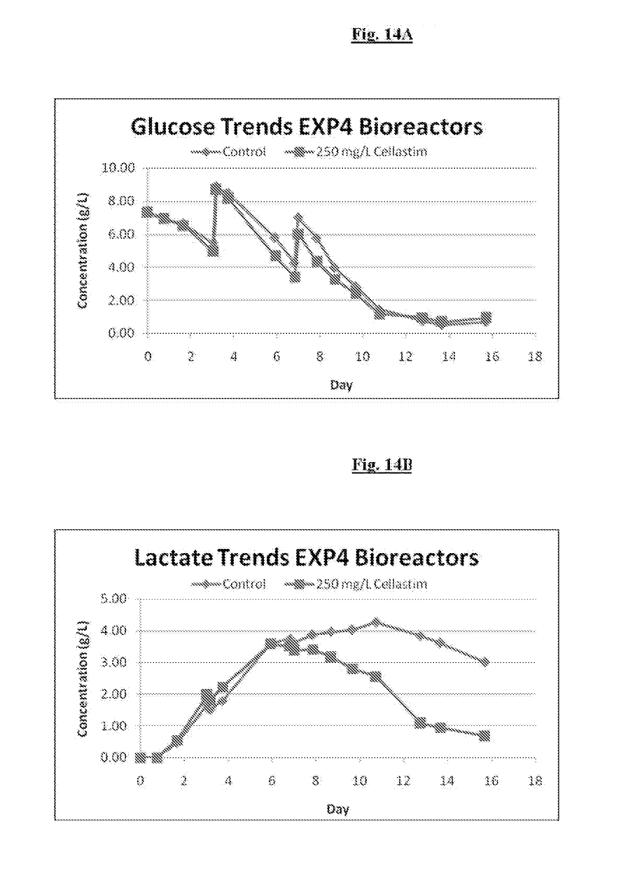

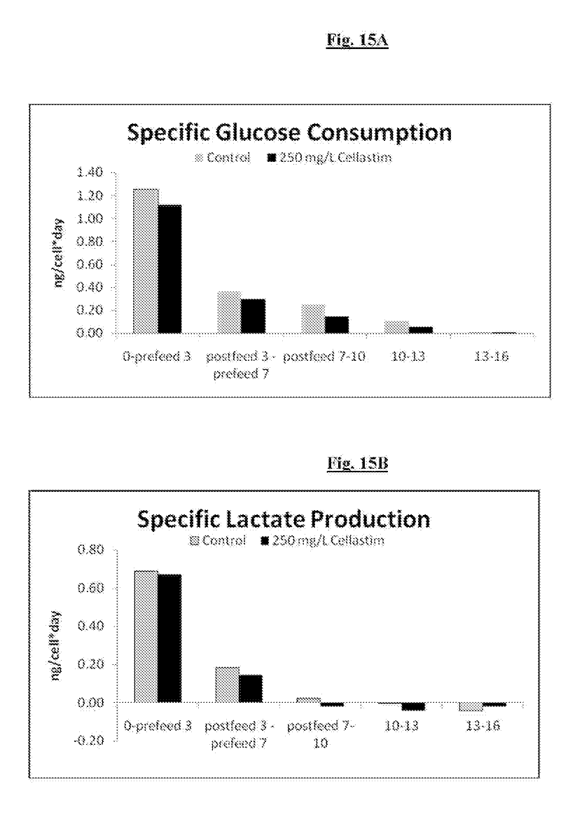

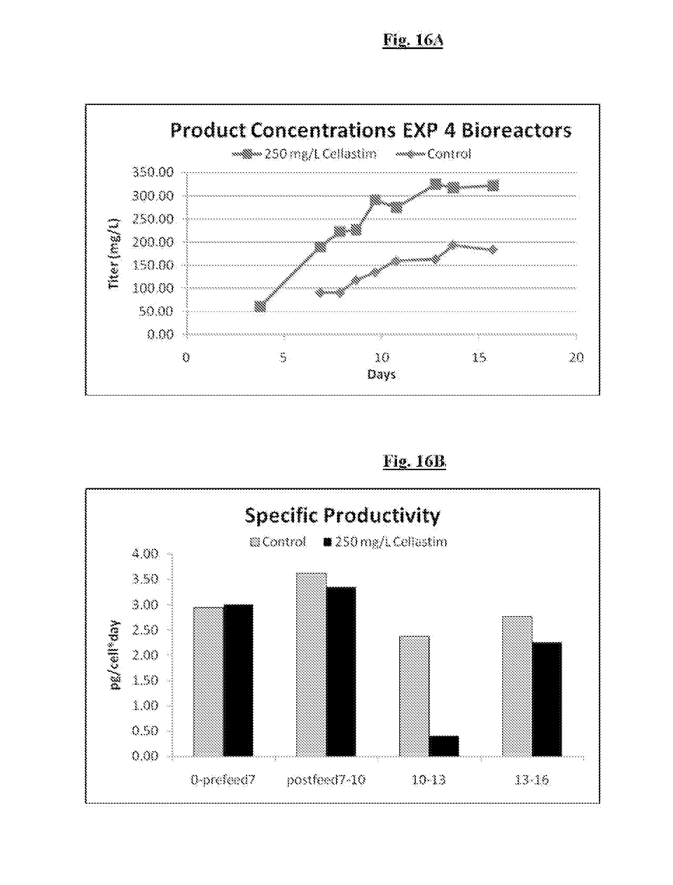

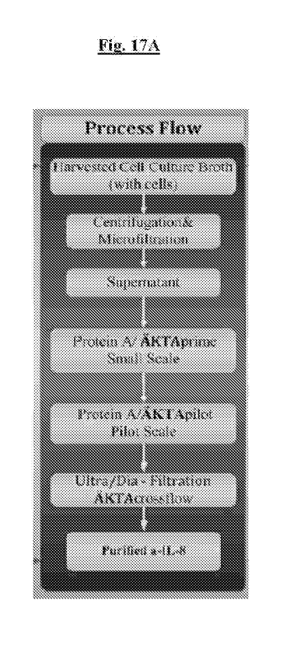

FIG. 6A shows the viable cell density VCD of cells grown in supplemented or in unsupplemented (control) base medium in shake flasks. In this experiment, cells were seeded in the base medium or in medium containing supplements in 30 ml of medium in 125 ml/shake flasks (Corning #431405) at a concentration of 3.0×10⁵ viable cells/ml. Cells were maintained at 37° C., in a humidified CO₂ incubator, at 110 RPM for the length of the run, and grown in the presence or absence of the indicated concentrations of either Cellastim, or a 1:1 mixture of Cellastim and Lactoferrin from Day 0. The figure shows that cells grew to higher density and remained at higher density in medium with the supplements of the invention compared to unsupplemented medium. Surprisingly, in this experiment viability was maintained as well at a concentration of 250 mg/ml Cellastim, as it was at 500 mg/ml Cellastim. FIG. 6B shows the percentage of viable cells present in the shake flask (% viability). The data show that cells maintained higher viability when the supplements were present in the medium. Thus the supplements of the invention increased both the absolute viable cell density and percentage viability of the cells throughout the period of the experiment compared to control cells grown in the absence of supplement. FIG. 7A shows the specific growth rate of the cells in different phases of the growth curve in shake flasks in supplemented and unsupplemented control medium. Note that supplemented cells maintained a positive growth rate through days 0-8, whereas the specific net growth rate decreases significantly on days 5-8 in the un-supplemented cultures. FIG. 7B shows the specific net death rate of cells during 3 phases of the growth curve. Note that cells grown in unsupplemented medium reached maximum peak death during days 5-8. Cells grown in medium with supplement reached maximum death rate later, on days 9-10 compared to the unsupplemented control incubations.

### Example 9

### Effects of Supplement Feeding on Cell Viability & Density and Product Production

Methods:

Cells were seeded in the base medium or in medium containing supplements in 30 ml of medium in 125 ml/shake flasks (Corning #431405) at a concentration of 3.0×10⁵ viable cells/ml. Cells were maintained at 37° C., in a humidified CO₂ incubator, at 110 RPM for the length of the run, and grown in the presence or absence of the indicated concentrations of either Cellastim, or a 1:1 mixture of Cellastim and Lactoferrin. In this experiment supplements were added at day 0 and a nutrient boost (feed) was added on day 4 according to the instructions of the manufacturer (Efficient Feed A, Invitrogen).

Results:

The growth profile of CHO-K1 in unsupplemented and supplemented medium in shake flasks when boosted with nutrient feed on day 4 is shown in FIG. 8A. The graph shows that cells attained a higher cell density when grown in medium with supplements at day 16 compared to the unsupplemented controls. FIG. 8B shows the percentage of viable cells (% Viability) present in shake flasks when boosted with nutrient feed on day 4 compared to non supplemented controls. The data show that cells maintained higher viability when the supplements of the invention were present in the media used added to the nutrient feed on day 4. FIG. 9A shows the specific growth rate of the cells in different phases of the growth curve in the shake flask studies in supplemented (boosted with nutrient feed on day 4 compared to unsupplemented control flasks. Note that supplemented cells maintained a positive growth rate through days 0-8. FIG. 9B shows the specific net death rate of cells during 4 different phases of the growth curve (boosted with a nutrient feed on day 4). Note that cells grown in supplemented medium showed lower cell death on day 12-16. FIG. 9C shows the concentration of antibody product produced by CHO K1 grown in supplemented and unsupplemented control medium in shake flasks. Monoclonal Antibody (MAb) concentration in the medium was higher in supplemented medium. The concentration of antibody produced by the cells and secreted into the medium was determined by anti-human IgG ELISA according to their procedure (Bethyl Laboratories).

### Example 10

### Protection from Adverse Events

Methods:

Cells were seeded in the base medium or in medium containing supplements in 30 ml of medium in 125 ml/shake flasks (Corning #431405) at a concentration of 3.0×10⁵ viable cells/ml. Cells were maintained at 37° C., in a humidified CO₂ incubator, at 110 RPM for the length of the run, and grown in the presence or absence of the indicated concentrations of either Cellastim, or a 1:1 mixture of Cellastim and Lactoferrin. In this experiment an unexplained event caused cell death during the loading of the bioreactors with cells

Results:

FIGS. 10A and 10B show that the supplements protect the cells from adverse events during bioreactor operations. CHO K1 cells grown in supplemented medium survived the adverse event and grew to high density (FIG. 10A) and reached high viablility (FIG. 10B). Cells grown in unsupplemented control medium did not grow.

### Example 11

### The Activity of Supplements with Dual Nutrient Feed in Bioreactors

A series of experiments (Exp 4) were also conducted in bioreactors to compare cell growth and bioreactor performance when cells are grown in unsupplemented control medium compared to medium supplemented with 250 mg/L Cellastim.

Methods

Fed batch bioreactor experiments were conducted in 1 L or 2 L Applikon bioreactors (Applikon Biotechnology, NE) in base medium or in base medium with supplements, as described above. Cultures were boosted on day 3 and day 7 with nutrient feed (Efficient Feed A, Invitrogen) as instructed by the manufacturer. Thus, these data show the effect of the supplement in combination with a dual-boost feed strategy. In addition the pH was lowered from 7.2 to 6.8 with the first nutrient feed.

Results:

Cells grown in supplemented medium grew to higher maximum cell density than cells grown in unsupplemented medium (FIG. 11A) Cells reached a density of 11 million viable cells/ml with supplementation compared to 8 million viable cells/ml in control medium. The specific growth rate was calculated for different phases of the growth profile. As shown in FIG. 11B, supplementation increased the growth rate the most in the pre-feed period day 0-3. FIGS. 12A & 12B show the percentage of viable cells and the specific death rate of CHO K1 cells grown in bioreactors with supplemented and unsupplemented medium using a dual nutrient boost on day 3 and 7. Cells grown in supplemented maintained high viability for the majority through day 13 despite the higher density of cells. The specific death rate was also similar throughout through day 13 despite the higher density of cells. FIGS. 13A & 13B show the pH and osmolality trends for CHO K1 grown in bioreactors using supplemented and unsupplemented medium. Cells were fed on day 3, at which time the pH was lowered from 7.10 to 6.8. The pH was maintained at 6.8 with the second feed on day 7. FIG. 13A shows that the supplement did not adversely affect the adjustment of pH within the bioreactor and that pH control was maintained. FIG. 13B shows the osmolality trend of the cells grown in supplemented and unsupplemented medium. The osmolality of the supplemented medium was lower than unsupplemented medium and closer to normal osmolality of 300. This data shows that the supplement favorably resulted in lower osmolality. FIGS. 14 A & B show the glucose and lactate trends for CHO K1 grown in supplemented and unsupplemented medium in bioreactors (with a nutrient feed on day 3 and 7). Glucose levels were similar in supplemented and unsupplemented medium. However, the level of lactate was favorably lower in medium with supplements. FIGS. 15 A & B show the specific glucose consumption, and specific lactate consumption of CHO K1 cells grown in supplemented and unsupplemented medium in bioreactors with a nutrient feed on day 3 and 7. The data show that cells favorably consumed less glucose in supplemented medium. The data also show that the cells favorably produced less lactate in the supplemented medium. FIGS. 16A & B show the concentration of antibody produced and the specific productivity of antibody in CHO K1 cells grown in supplemented and unsupplemented medium in bioreactors with a nutrient feed on day 3 and 7. The concentration of produced antibody was significantly higher when cells were grown in supplemented medium. The specific production of antibody was similar when cells were grown in supplemented and unsupplemented medium.

### Example 12

### Comparison of Adding the Supplement at Inoculum or with a Nutrient Feed (EXP4 Shake Flask Studies)

Methods:

CHO K1 cells were seeded into medium as described above at 3.0×105 viable cells/ml in 30 ml of medium in 125 ml shake flasks. In these experiments cells were fed with nutrient feed at day 3 and 7 (Efficient Feed A, Invitrogen, as instructed by the manufacturer). Supplement was added either at day 0 with the innoculum of cells, or on day 3 with the first nutrient feed.

Results:

The data (not shown) indicated that there is little difference in glucose levels, osmolality, pH, lactate levels or production, glucose levels or glucose consumption whether the supplement is added at day 0 vs day 3. There was however a modest improvement in productivity at the beginning late phase (day 11) when the supplement was added with the innoculum. Table E7 shows the percent improvement product produced by CHO K1 when supplement was added either on day 0 or with the feed on day 3. More antibody was produced with the supplements, compared to no supplements, and adding the supplement with the cell innoculum produced more product compared to adding supplement with the feed.

TABLE E7

| | Incubation Conditions | | | % Improvement |
|---|---|---|---|---|
| | 250 | 250 | | (250 Inoculation/ |
| Day | Inoculation | Feed | Control | Control) |

Product Concentration

| | | | | |
|---|---|---|---|---|
| Day 11 | 334.8 | 299.6 | 290.6 | 15.2% |
| Day 16 | 338.7 | 340 | 332 | 2.0% |

Volumetric Productivity

| | | | |
|---|---|---|---|
| Day 11 | 30.4 | 27.2 | 26.4 |
| Day 16 | 21.2 | 21.3 | 20.8 |
| % Improvement | 43.8% | 28.2% | 27.3% |
| (Day 11/Day 16) | | | |

Table E8 shows the percent improvement seen with supplemented medium in various experiments in shake flasks and bioreactor culture systems.

TABLE E8
Percent improvement seen (% Improvement, Supplemented/Control)

| | |
|---|---|
| Peak viable cell density (VCD): | Average: 28%, Range: 2-47% |

### Example 13

### Impact of Cellastim and Lacromin on Downstream Antibody Purification

The following data shows that Cellastim and recombinant human Lactoferrin (Lacromin) when used as media supplements, had a positive effect on the yield and overall purity of antibody recovered from the cell supernatant.

Methods: CHO K1 cells producing an antibody to interleukin 8 (a-IL*) were grown medium with supplements or without supplement as described above. Control cultures used unsupplemented medium. Cells were grown in medium with either of 3 supplements: 1) 250 mg/L Cellastim 2) 500 mg/L Cellastim, or 3), 125 mg/L Cellastim and 125 mg/L Lactoferrin (Lacromin).

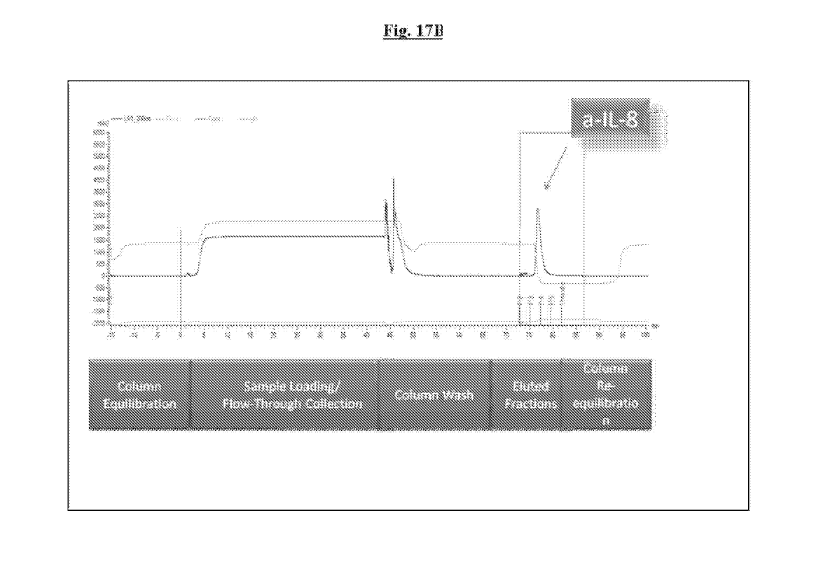

As shown schematically in FIG. 17A, medium was harvested from cells at the end of batch when cell viability reached 80-50%. Particulate cell debris was first removed from harvested cell culture broth by centrifugation and microfiltration though a 0.2 micro filter. The filtrate (supernatant) was processed over either of two sizes of protein A columns: GE-ÄKTAprime (small scale affinity chromatography system with 1 ml Protein A chromatography column (GE Healthcare HiTrap™ MabSelect™ SuRe) for pre-/post AKTA Pilot sample testing or GE-ÄKTApilot (affinity chromatography system with 100 ml Protein A chromatography column (GE Healthcare XK50 MABSELECT™ SuRe). Results of the elution profile are shown in FIG. 17B.

Following protein A chromatography, the eluted antibody was concentrated by Dia-filtration using an ÄKTAcrossflow apparatus with 10 kD GE KVICK™ Start polyethersulfone membrane as described by the manufacturer. Following purification, the antibody was analyzed by SDS-PAGE to detect impurities and the presence of target protein utilizing Coomassie Blue and Silver Staining.

Results:

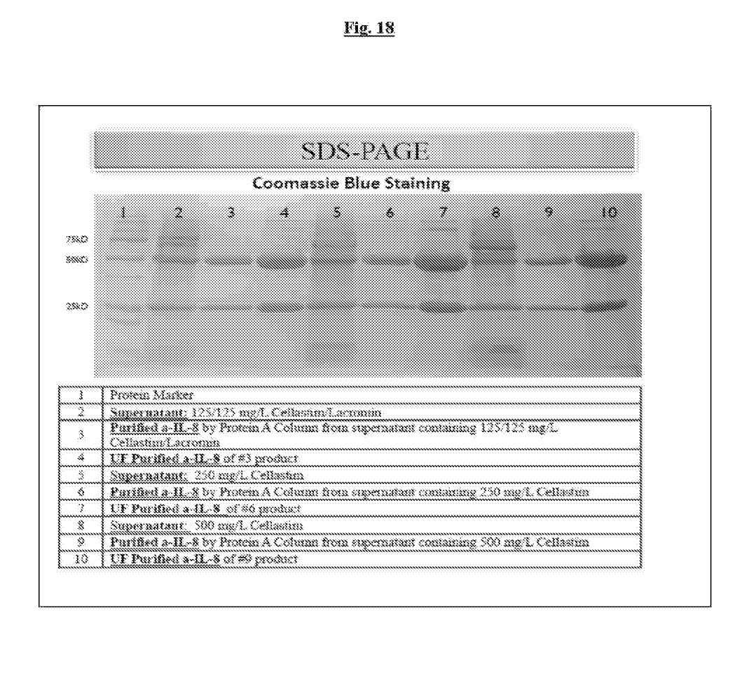

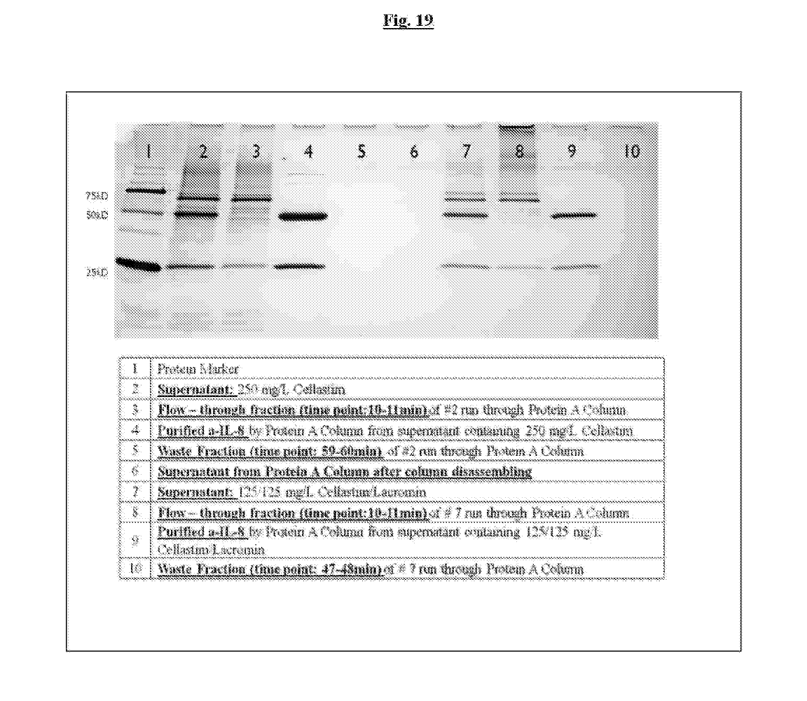

FIG. 18 shows the SDS-PAGE analysis of various fractions with Coomassie blue staining showing the purification of antibody and the successful removal of the media supplements by protein A chromatography. FIG. 19 shows SDS-PAGE analysis of various fractions with silver staining showing the purification of antibody and the successful removal of the media supplements by protein A chromatography. In all cases of supplementation, purification of the antibody is enhanced. These gels clearly establish that the use of the supplements of the invention leads to i) higher yields of product, ii) product that is enriched in correctly folded forms; specifically correctly assembled multimeric antibody heavy and light chains, iii) product that is of a higher purity, iv) product that is less contaminated with other endogenous cellular proteins and iv) product that is less degraded by cellular proteases, compared to batches of product made without the supplements of the present invention. Additionally as shown below in Table E9, the recovery of antibody from harvested medium is also improved with the supplements in the medium.

TABLE E9

| | Amount of IgG | Amount of IgG | % |
|---|---|---|---|
| Sample | loaded (mg) | recovered (mg) | Recovered |
| Control | 784 | 328 | 41.8 |
| 0 mg/L Cellastim | | | |
| 0 mg/L Lacromin | | | |
| 125 mg/L Cellastim | 619 | 321 | 51.9 |
| 125 mg/L Lacromin | | | |
| 250 mg/L Cellastim | 537 | 315 | 58.7 |
| 500 mg/L Cellastim | 449 | 313 | 69.7 |

These data show that the supplements can be used at different concentrations and in combination with different media compositions and can have a positive effect of recovery-without negatively affecting the purity of the product recovery after protein A chromatography. Importantly this example demonstrates that the supplements of the present invention provides for superior methods for improving product recovery during the purification process, and improved product purifications, with products containing less contaminating cellular proteins during each step of purification. Such products are anticipated to exhibit improved bioactivity, stability and to be less immunogenic and allogenic compared to product made without the supplements of the present invention.

1-72. (canceled)

73. A method for enhancing the growth of a cell growing in cell culture medium, comprising adding a supplement comprising recombinant albumin to said cell culture medium,
wherein said recombinant albumin is produced in a plant;
wherein said supplement has less than about 1 EU of endotoxin per mg of albumin; and
wherein said albumin comprises less than about 2% aggregated albumin.

74. A method for enhancing the productivity of a cell that has been adapted to serum free media, comprising adding a supplement comprising recombinant albumin to said serum free media,
wherein said recombinant albumin is produced in a plant;
wherein said supplement has less than about 1 EU of endotoxin per mg of albumin; and
wherein said albumin comprises less than about 2% aggregated albumin.

75. The method of claim 73 or 74, wherein said cell is a tissue culture cell.

76. The method of claim 73 or 74, wherein said cell is selected from the group consisting of a CHO cell, a hybridoma cell, a Vero cell, or a primary cell.

77. The method of claim 76, wherein said primary cell is a stem cell.

78. The method of claim 76, wherein said primary cell is B-cell derived.

79. The method of claim 76, wherein said primary cell is a B-cell.

80. The method of claim 76, wherein said primary cell is T-cell derived.

81. The method of claim 76, wherein said primary cell is a T-cell.

82. The method of claim 73 or 74, wherein said supplement comprises at least about 0.01% wt/wt of a heat shock protein.

83. The method of claim 82, wherein said heat shock protein is a rice heat shock protein.

84. The method of claim 83, wherein said rice heat shock protein is selected from the group consisting of rice HSP70 and rice endosperm lumenal binding protein.

85. The method of claim 83, wherein said rice heat shock protein is selected from the group consisting of rice (gblACJ54890.1l), EEC69073/OsI_{—}37938, and AAB63469.

86. The method of claim 84, wherein said supplement comprises at least about 0.01% wt/wt HSP 70, at least about 0.04% wt/wt HSP 70, at least about 0.06% wt/wt HSP 70, at least about 0.08% wt/wt HSP 70, or at least about 0.1% wt/wt HSP 70.

87. The method of claim 73 or 74, wherein said recombinant albumin is added to a final concentration of between about 100 mg/L and about 200 mg/L, between about 200 mg/L and about 400 mg/L, between about 400 mg/L and about 600 mg/L, between about 600 mg/L and about 800 mg/L, between about 800 mg/L and about 1000 mg/L, between about 1000 mg/L and about 2000 mg/L, between about 2000 mg/L and about 5000 mg/L, between about 5000 mg/L and about 10000 mg/L, or between about 10000 mg/L and about 20000 mg/L.
